(19) United States
(12) Patent Application Publication
(10) Pub. No.: US 2024/0100099 A1
(43) Pub. Date: Mar. 28, 2024

(54) COMPOSITION FOR REGENERATION OF INTERVERTEBRAL DISC

(71) Applicants: National University Corporation Hokkaido University, Sapporo-shi, Hokkaido (JP); PuREC Co., Ltd., Izumo-shi, Shimane (JP); Mochida Pharmaceutical Co., Ltd., Shinjuku-ku, Tokyo (JP)

(72) Inventors: Hideki SUDO, Sapporo-shi, Hokkaido (JP); Daisuke UKEBA, Sapporo-shi, Hokkaido (JP); Katsuhisa YAMADA, Sapporo-shi, Hokkaido (JP); Katsuro URA, Sapporo-shi, Hokkaido (JP); Hisataka SUZUKI, Sapporo-shi, Hokkaido (JP); Yumi IYOKU, Izumo-shi, Shimane (JP); Takashi SUYAMA, Izumo-shi, Shimane (JP)

(73) Assignees: National University Corporation Hokkaido University, Sapporo-shi, Hokkaido (JP); PuREC Co., Ltd., Izumo-shi, Shimane (JP); Mochida Pharmaceutical Co., Ltd., Shinjuku-ku, Tokyo (JP)

(21) Appl. No.: 18/273,654

(22) PCT Filed: Jan. 28, 2022
(86) PCT No.: PCT/JP2022/004342

§ 371 Date: Jul. 21, 2023

(30) Foreign Application Priority Data
Jan. 29, 2021 (JP) 2021-013667
Oct. 13, 2021 (JP) 2021-167913

(51) Int. Cl. A61K 35/28; A61K 9/00; A61K 47/36; A61P 19/02

(52) U.S. Cl. CPC A61K 35/28; A61K 9/0019; A61K 47/36; A61P 19/02

(57) ABSTRACT

Provided is a composition for promotion of the regeneration of the nucleus pulposus of an intervertebral disc, said composition comprising a low endotoxin monovalent metal salt of alginic acid and mesenchymal stem cells. In particular, the composition of the present invention promotes the regeneration of the nucleus pulposus of an intervertebral disc via activation of nucleus pulposus cells by human bone marrow-derived high-purity mesenchymal stem cells and/or differentiation of human bone marrow-derived high-purity mesenchymal stem cells into nucleus pulposus cells.

### TECHNICAL FIELD

The present invention relates to a composition for regeneration of an intervertebral disc, comprising a low endotoxin monovalent metal salt of alginic acid and mesenchymal stem cells. The present invention relates to a composition for regeneration of an intervertebral disc, comprising human bone marrow-derived high-purity mesenchymal stem cells.

### BACKGROUND ART

The human backbone (spine) consists of 24 bones (vertebrae), and the tissues that play a role as a cushion between a vertebra and another vertebra is referred to as an “intervertebral disc.” The intervertebral discs are degenerated and damaged due to aging, trauma, disease, and the like. Degeneration of intervertebral discs is a state in which the number of cells, water contents, extracellular matrixes (type II collagen, aggrecan, etc.), and the like in intervertebral discs are reduced, and as this state progresses, the intervertebral discs are unable to function as shock absorbers. Specific examples of the intervertebral disc degeneration and the intervertebral disc damage may include intervertebral disc herniation, discopathy, degenerative spondylolisthesis, pyogenic discitis, spondylosis deformans, spinal canal stenosis, and intervertebral disc injuries due to trauma and the like. For example, in the case of intervertebral disc herniation, the annulus fibrosis covering the nucleus pulposus is deformed or fissured to form a hernia, and the hernia protrudes outside the intervertebral disc, and also, the deformed nucleus pulposus or the nucleus pulposus that is deviated from the generated fissure compresses the spinal nerves, causing pain, paralysis, etc.

One of treatments for intervertebral disc herniation is discectomy (resection), which has been proved to be effective to some extent. However, it has been known that, according to such discectomy (resection), degenerative changes of the intervertebral discs may progress because the surgical site is not treated after discectomy. When a part of the nucleus pulposus is removed by discectomy, a cavity (which is also referred to as a “defective site” in the present description) is created at the nucleus pulposus site. Since the self-repairing ability and regeneration ability of the nucleus pulposus are extremely low, the cavity of the nucleus pulposus tends to be physically weakened. In addition, fibroblast-like cells may accumulate in such a cavity part, and tissues having mechanical properties different from those of the original nucleus pulposus may be formed. For this reason, after discectomy, herniation recurs at a high recurrence rate. The recurrence rate of herniation within 5 years after discectomy is said to be approximately 4% to 15%. According to recent long-term data, it has been found that herniation recurs in the majority of patients after 10 years. If herniation recurs, reoperation is required. However, the spinal nerves are buried in the scar tissues formed after the first surgery, and thus, it becomes difficult to confirm the position of the spinal nerves.

To date, high-purity curable gels have been developed based on alginic acid, and it has been demonstrated that intervertebral disc tissues can be spontaneously repaired by single administration of the gel (cell-free) (Japanese Patent No. 6487110, Tsujimoto et al., EBioMedicine 37 (2018) 521-534). A clinical trial of gel implantation after herniectomy is being conducted on lumbar intervertebral disc herniation patients aged 20 to 49 years.

By the way, mesenchymal stem cells (MSCs) cause few ethical issues associated with cell collection, and have differentiation ability to differentiate into various organs such as bones, cartilages and fats, and thus, MSCs are somatic stem cells that are frequently used in clinical sites, after hematopoietic stem cells. Since MSCs can be isolated by relatively simple procedures, these cells are widely used as materials for regenerative medicine, and mainly, MSCs are used for local transplantation after the cells have been induced to differentiate into cartilages, bones, and the like in vitro. In order to promote clinical application of MSCs, it is essential for productization of MSCs to be able to produce the cells that maintain certain functions in a necessary amount.

The present inventors have separated LNGFR and Thy-1 double-positive cells from a bone marrow fluid according to flow cytometry, and have obtained rapidly expanding clones (RECs). The present inventors have established a purification and separation method capable of excluding a difference in proliferative ability of MSCs derived from donors (Japanese Patent No. 6463029, WO2016/017795, Mabuchi Y. et al., Stem Cell Reports 1(2): 152-165, 2013).

### CITATION LIST

### Patent Literature

[PTL 1] Japanese Patent No. 6487110
[PTL 2] Japanese Patent No. 6463029
[PTL 3] International Publication WO2016/017795

### Non Patent Literature

[NPL 1] Tsujimoto T et al., An acellular bioresorbable ultra-purified alginate gel promotes intervertebral disc repair: A preclinical proof-of-concept study, EBioMedicine 37 (2018) 521-534
[NPL 2] Mabuchi Y et al., LNGFR+Thy-1+Vcam-1hi+ cells reveal functionally distinct subpopulations in mesenchymal stem cells. Stem Cell Reports 1(2): 152-165, 2013

### SUMMARY OF INVENTION

### Technical Problem

As described above, to date, high-purity curable gels have been developed based on alginic acid, and an exploratory, investigator-initiated trial regarding gel implantation after herniectomy has been performed on patients with lumbar intervertebral disc herniation. Nevertheless, the nucleus pulposus, which is considered to have almost no self-repairing ability and regeneration ability, becomes further poor in such self-repairing ability in middle-aged and older age. For such middle-aged and older patients, gel monotherapy has obvious limitations.

Under such circumstances, it has been desired to further develop a composition capable of repairing and/or regenerating intervertebral disc tissues.

### Solution to Problem

As a result of studies conducted directed towards achieving the aforementioned object, the present inventor has found that a composition comprising a low endotoxin monovalent metal salt of alginic acid and mesenchymal stem cells can achieve the aforementioned object, thereby completing the present invention. Moreover, in another aspect of the present invention, the present inventor has found that a composition comprising human bone marrow-derived high-purity mesenchymal stem cells can achieve the aforementioned object.

Specifically, the present invention is as follows.
[1] A composition for regeneration of an intervertebral disc, comprising a low endotoxin monovalent metal salt of alginic acid and mesenchymal stem cells.
[1-1] A composition for regeneration of an intervertebral disc, comprising a monovalent metal salt of alginic acid and mesenchymal stem cells.
[2] The composition according to [1] or [1-1], wherein regeneration of the nucleus pulposus of an intervertebral disc is promoted via activation of nucleus pulposus cells by mesenchymal stem cells and/or differentiation of mesenchymal stem cells into nucleus pulposus cells.
[3] The composition according to any one of [1] to [2], wherein the mesenchymal stem cells are human bone marrow-derived high-purity mesenchymal stem cells.
[4] The composition according to [3], wherein the human bone marrow-derived high-purity mesenchymal stem cells are a cell population of LNGFR (CD271)-positive, or LNGFR (CD271) and Thy-1 (CD90) double-positive, rapidly proliferating mesenchymal stem cell clones, said cell population satisfying at least one of the following characteristics (a) and (b):
   (a) the coefficient of variation of forward scattered light in flow cytometry is 35% or less; and
   (b) the average cell size is 20 lam or less.
[4-1] The composition according to [3], wherein the human bone marrow-derived high-purity mesenchymal stem cells are a cell population of rapidly proliferating mesenchymal stem cell clones that are separated using, as an indicator, the feature that the cells are LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 35% or less; and
   (b) the average cell size is 20 lam or less.
[4-2] The composition according to [3], wherein the human bone marrow-derived high-purity mesenchymal stem cells are a cell population of rapidly proliferating mesenchymal stem cell clones that are derived from LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 35% or less; and
   (b) the average cell size is 20 lam or less.
[4-3] The composition according to [3], wherein the human bone marrow-derived high-purity mesenchymal stem cells are a cell population of LNGFR (CD271)-positive, or LNGFR (CD271) and Thy-1 (CD90) double-positive, rapidly proliferating mesenchymal stem cell clones, said cell population satisfying at least one of the following characteristics (a) and (b):
   (a) the coefficient of variation of forward scattered light in flow cytometry is 40% or less; and
   (b) the average cell size is 20 lam or less.
[4-4] The composition according to [3], wherein the human bone marrow-derived high-purity mesenchymal stem cells are a cell population of rapidly proliferating mesenchymal stem cell clones that are separated using, as an indicator, the feature that the cells are LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 40% or less; and
   (b) the average cell size is 20 lam or less.
[4-5] The composition according to [3], wherein the human bone marrow-derived high-purity mesenchymal stem cells are a cell population of rapidly proliferating mesenchymal stem cell clones that are derived from LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 40% or less; and
   (b) the average cell size is 20 lam or less.
[5] The composition according to any one of [1] to [4-5], which is applied to the intervertebral disc of a subject and has fluidity upon the application thereof
[5-1] The composition according to any one of [1] to [4], wherein the composition is used such that the composition is applied to the nucleus pulposus site and a crosslinking agent is brought into contact with at least a part of a surface of the composition after the application, and the composition has fluidity when applied to the nucleus pulposus site.
[5-2] The composition according to any one of [1] to [4], wherein the composition is used such that the composition is applied to the nucleus pulposus site and is partially cured after the application, and the composition has fluidity when applied to the nucleus pulposus site.
[6] The composition according to any one of [5] to [5-2], wherein the application to the nucleus pulposus site is the filling of the composition into a defective site of the nucleus pulposus.
[7] The composition according to [5-2] or [6], wherein the curing of a part of the composition is carried out by bringing a crosslinking agent into contact with at least a part of the surface of the composition.
[8] The composition according to [5-1] or [7], wherein the crosslinking agent is a divalent or higher valent metal ion compound.
[8-1] The composition according to any one of [1] to [8], wherein the monovalent metal salt of alginic acid is a low endotoxin monovalent metal salt of alginic acid.
[9] The composition according to any one of [1] to [8-1], wherein the low endotoxin monovalent metal salt of alginic acid has a weight average molecular weight (absolute molecular weight) of 80,000 or more, as measured by a GPC-MALS method.
[10] The composition according to any one of [1] to [9], wherein the concentration of the low endotoxin monovalent metal salt of alginic acid is 0.5 w/w % to 5.0 w/w %.
[11] The composition according to any one of [1] to [10], which is for use in the treatment, prevention or recurrence suppression of intervertebral disc degeneration and/or intervertebral disc damage.
[12] The composition according to [11], wherein the intervertebral disc degeneration and/or the intervertebral disc damage are at least one selected from the group consisting of intervertebral disc herniation, discopathy, degenerative spondylolisthesis, pyogenic discitis, spondylosis deformans, spinal canal stenosis, lumbar spinal stenosis, intervertebral disc herniation associated with lumbar spinal stenosis, and intervertebral disc damage.
[12-1] The composition according to or [12], wherein the intervertebral disc degeneration and/or the intervertebral disc damage are associated with chronic low back pain.
[13] A composition for regeneration of an intervertebral disc, comprising human bone marrow-derived high-purity mesenchymal stem cells, wherein the composition is applied to the nucleus pulposus site of the intervertebral disc of a subject.
[14] The composition according to [13], wherein regeneration of the nucleus pulposus of an intervertebral disc is promoted via activation of nucleus pulposus cells by human bone marrow-derived high-purity mesenchymal stem cells and/or differentiation of human bone marrow-derived high-purity mesenchymal stem cells into nucleus pulposus cells.
[15] The composition according to or [14], wherein the human bone marrow-derived high-purity mesenchymal stem cells are in an undifferentiated state upon the application thereof and/or are applied without treatments of induction of differentiation.
[16] The composition according to any one of to [15], wherein the human bone marrow-derived high-purity mesenchymal stem cells are a cell population of LNGFR (CD271)-positive, or LNGFR (CD271) and Thy-1 (CD90) double-positive, rapidly proliferating mesenchymal stem cell clones, said cell population satisfying at least one of the following characteristics (a) and (b):
   (a) the coefficient of variation of forward scattered light in flow cytometry is 35% or less; and
   (b) the average cell size is 20 lam or less.
[16-1] The composition according to any one of to [15], wherein the human bone marrow-derived high-purity mesenchymal stem cells are a cell population of rapidly proliferating mesenchymal stem cell clones that are separated using, as an indicator, the feature that the cells are LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 35% or less; and
   (b) the average cell size is 20 μm or less.
[16-2] The composition according to any one of to [15], wherein the human bone marrow-derived high-purity mesenchymal stem cells are a cell population of rapidly proliferating mesenchymal stem cell clones that are derived from LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 35% or less; and
   (b) the average cell size is 20 μm or less.
[16-3] The composition according to any one of to [15], wherein the human bone marrow-derived high-purity mesenchymal stem cells are a cell population of LNGFR (CD271)-positive, or LNGFR (CD271) and Thy-1 (CD90) double-positive, rapidly proliferating mesenchymal stem cell clones, said cell population satisfying at least one of the following characteristics (a) and (b):
   (a) the coefficient of variation of forward scattered light in flow cytometry is 40% or less; and
   (b) the average cell size is 20 μm or less.
[16-4] The composition according to any one of to [15], wherein the human bone marrow-derived high-purity mesenchymal stem cells are a cell population of rapidly proliferating mesenchymal stem cell clones that are separated using, as an indicator, the feature that the cells are LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 40% or less; and
   (b) the average cell size is 20 μm or less.
[16-5] The composition according to any one of to [15], wherein the human bone marrow-derived high-purity mesenchymal stem cells are a cell population of rapidly proliferating mesenchymal stem cell clones that are derived from LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 40% or less; and
   (b) the average cell size is 20 μm or less.
[17] The composition according to any one of to [16-5], further comprising a carrier for embedding cells.
[18] The composition according to [17], wherein the carrier is selected from the group consisting of alginic acid, hyaluronic acid, chondroitin sulfate, dermatan sulfate, keratan sulfate, heparin, heparan sulfate, galactosaminoglycuron glycan sulfate, and pharmaceutically acceptable salts thereof
[19] The composition according to [17], wherein the carrier is a monovalent metal salt of alginic acid.
[20] The composition according to any one of to [19], which is used such that the composition is applied to the nucleus pulposus site of the intervertebral disc of a subject and, after the application thereof, a crosslinking agent is brought into contact with at least a part of the surface of the composition, and which has fluidity upon the application thereof to the nucleus pulposus site.
[21] The composition according to any one of to [19], which is used such that the composition is applied to the nucleus pulposus site of the intervertebral disc of a subject, and a part of the composition is cured after the application thereof, and which has fluidity upon the application thereof to the nucleus pulposus site.
[22] The composition according to or [21], wherein the application to the nucleus pulposus site is the filling of the composition into a defective site of the nucleus pulposus.
[23] The composition according to any one of to [22], wherein the crosslinking agent is a divalent or higher valent metal ion compound.
[24] The composition according to any one of to [23], wherein the monovalent metal salt of alginic acid has a weight average molecular weight (absolute molecular weight) of 80,000 or more, as measured by a GPC-MALS method.
[25] The composition according to any one of to [24], wherein the concentration of the monovalent metal salt of alginic acid is 0.5 w/w % to 5.0 w/w %.
[26] The composition according to any one of to [25], wherein the monovalent metal salt of alginic acid is a low endotoxin monovalent metal salt of alginic acid.
[27] The composition according to any one of to [26], which is for use in the treatment, prevention, or recurrence suppression of intervertebral disc degeneration and/or intervertebral disc damage.
[28] The composition according to [27], wherein the intervertebral disc degeneration and/or the intervertebral disc damage are at least one selected from the group consisting of intervertebral disc herniation, discopathy, degenerative spondylolisthesis, pyogenic discitis, spondylosis deformans, spinal canal stenosis, lumbar spinal stenosis, intervertebral disc herniation associated with lumbar spinal stenosis (combined lumbar spinal stenosis), and intervertebral disc damage.
[28-1] The composition according to or [28], wherein the intervertebral disc degeneration and/or the intervertebral disc damage are associated with chronic low back pain.
[29] The composition according to any one of [1] to [28-1], which is used to suppress intervertebral disc pain.
[30] The composition according to [29], wherein the intervertebral disc pain is chronic low back pain.
[31] A composition for regeneration of an intervertebral disc, comprising a monovalent metal salt of alginic acid and human bone marrow-derived high-purity mesenchymal stem cells, wherein the composition is applied to the nucleus pulposus site of the intervertebral disc of a subject, in a state in which the composition has fluidity.
[31-1] The composition for regeneration of an intervertebral disc according to [31], which is used without requiring treatments of curing the composition, after the composition is applied to the nucleus pulposus site of the intervertebral disc of a subject, in a state in which the composition has fluidity.
[31-2] The composition for regeneration of an intervertebral disc according to or [31-1], which is used without bringing a crosslinking agent into contact with the composition, after the composition is applied to the nucleus pulposus site of the intervertebral disc of a subject, in a state in which the composition has fluidity.
[32] The composition according to any one of to [31-2], wherein regeneration of the nucleus pulposus of an intervertebral disc is promoted via activation of nucleus pulposus cells by human bone marrow-derived high-purity mesenchymal stem cells and/or differentiation of human bone marrow-derived high-purity mesenchymal stem cells into nucleus pulposus cells.
[33] The composition according to any one of to [32], wherein the human bone marrow-derived high-purity mesenchymal stem cells are in an undifferentiated state upon the application thereof and/or are applied without treatments of induction of differentiation.
[34] The composition according to any one of to [33], wherein the human bone marrow-derived high-purity mesenchymal stem cells are a cell population of LNGFR (CD271)-positive, or LNGFR (CD271) and Thy-1 (CD90) double-positive, rapidly proliferating mesenchymal stem cell clones, said cell population satisfying at least one of the following characteristics (a) and (b):
   (a) the coefficient of variation of forward scattered light in flow cytometry is 40% or less; and
   (b) the average cell size is 20 μm or less.
[34-1] The composition according to any one of to [33], wherein the human bone marrow-derived high-purity mesenchymal stem cells are a cell population of rapidly proliferating mesenchymal stem cell clones that are separated using, as an indicator, the feature that the cells are LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 40% or less; and
   (b) the average cell size is 20 μm or less.
[34-2] The composition according to any one of to [33], wherein the human bone marrow-derived high-purity mesenchymal stem cells are a cell population of rapidly proliferating mesenchymal stem cell clones that are derived from LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 40% or less; and
   (b) the average cell size is 20 μm or less.
[34-3] The composition according to any one of to [34-2], wherein the coefficient of variation is 35% or less.
[35] The composition according to any one of to [34], wherein the application to the nucleus pulposus site is the filling of the composition into a defective site of the nucleus pulposus.
[36] The composition according to any one of to [35], wherein the monovalent metal salt of alginic acid has a weight average molecular weight (absolute molecular weight) of 80,000 or more, as measured by a GPC-MALS method.
[37] The composition according to any one of to [36], wherein the concentration of the monovalent metal salt of alginic acid is 0.5 w/w % to 5.0 w/w %.
[38] The composition according to any one of to [37], wherein the monovalent metal salt of alginic acid is a low endotoxin monovalent metal salt of alginic acid.
[39] The composition according to any one of to [38], which is for use in the treatment, prevention, or recurrence suppression of intervertebral disc degeneration and/or intervertebral disc damage.
[40] The composition according to [39], wherein the intervertebral disc degeneration and/or the intervertebral disc damage are at least one selected from the group consisting of intervertebral disc herniation, discopathy, degenerative spondylolisthesis, pyogenic discitis, chronic low back pain, spondylosis deformans, spinal canal stenosis, lumbar spinal stenosis, intervertebral disc herniation associated with lumbar spinal stenosis (combined lumbar spinal stenosis), and intervertebral disc damage.
[41] The composition according to or [40], wherein the intervertebral disc degeneration and/or the intervertebral disc damage are associated with chronic low back pain.
[42] The composition according to any one of to [41], which is used to suppress intervertebral disc pain.
[43] The composition according to [42], wherein the intervertebral disc pain is chronic low back pain.
[44] A composition for suppression of intervertebral disc pain, comprising a monovalent metal salt of alginic acid and human bone marrow-derived high-purity mesenchymal stem cells, wherein the composition is applied to in the nucleus pulposus site of a subject, in a state in which the composition has fluidity.
[44-1] The composition for suppression of intervertebral disc pain according to [44], which is used without requiring treatments of curing the composition, after the composition is applied to the nucleus pulposus site of the intervertebral disc of a subject, in a state in which the composition has fluidity.
[44-2] The composition for suppression of intervertebral disc pain according to or [44-1], which is used without bringing a crosslinking agent into contact with the composition, after the composition is applied to the nucleus pulposus site of the intervertebral disc of a subject, in a state in which the composition has fluidity.
[45] The composition according to any one of to [44-2], wherein the pain is associated with at least one of the group consisting of intervertebral disc herniation, discopathy, degenerative spondylolisthesis, pyogenic discitis, spondylosis deformans, spinal canal stenosis, lumbar spinal stenosis, intervertebral disc herniation associated with lumbar spinal stenosis (combined lumbar spinal stenosis), and intervertebral disc damage.
[46] The composition according to any one of to [45], wherein the pain is chronic low back pain.
[47] The composition according to any one of to [46], wherein the human bone marrow-derived high-purity mesenchymal stem cells are in an undifferentiated state upon the application thereof and/or are applied without treatments of induction of differentiation.
[48] The composition according to any one of to [47], wherein the human bone marrow-derived high-purity mesenchymal stem cells are a cell population of LNGFR (CD271)-positive, or LNGFR (CD271) and Thy-1 (CD90) double-positive, rapidly proliferating mesenchymal stem cell clones, said cell population satisfying at least one of the following characteristics (a) and (b):
   (a) the coefficient of variation of forward scattered light in flow cytometry is 40% or less; and
   (b) the average cell size is 20 nm or less.
[48-1] The composition according to any one of to [47], wherein the human bone marrow-derived high-purity mesenchymal stem cells are a cell population of rapidly proliferating mesenchymal stem cell clones that are separated using, as an indicator, the feature that the cells are LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 40% or less; and
   (b) the average cell size is 20 nm or less.
[48-2] The composition according to any one of to [47], wherein the human bone marrow-derived high-purity mesenchymal stem cells are a cell population of rapidly proliferating mesenchymal stem cell clones that are derived from LNGFR (CD271)-positive, or LNGFR (CD271) and Thy-1 (CD90) double-positive cells, said cell population satisfying at least one of the following characteristics (a) and (b):
   (a) the coefficient of variation of forward scattered light in flow cytometry is 40% or less; and
   (b) the average cell size is 20 nm or less.
[48-3] The composition according to any one of to [48-2], wherein the coefficient of variation is 35% or less.
[48-4] The composition according to any one of to [48-3], wherein the application to the nucleus pulposus site is the filling of the composition into a defective site of the nucleus pulposus.
[49] The composition according to any one of to [48-4], wherein the monovalent metal salt of alginic acid has a weight average molecular weight (absolute molecular weight) of 80,000 or more, as measured by a GPC-MALS method.
[50] The composition according to any one of to [49], wherein the concentration of the monovalent metal salt of alginic acid is 0.5 w/w % to 5.0 w/w %.
[51] The composition according to any one of to [50], wherein the monovalent metal salt of alginic acid is a low endotoxin monovalent metal salt of alginic acid.
[52] The composition according to any one of to [51], which is applied to the nucleus pulposus site of the intervertebral disc of a subject under x-ray fluoroscopy, which has fluidity upon the application thereof to the nucleus pulposus site, and which is used without bringing a crosslinking agent into contact with the composition.
[53] The composition according to [52], which is applied to the nucleus pulposus site of the intervertebral disc of a subject under x-ray fluoroscopy, using a pressure gauge syringe, which has fluidity upon the application thereof to the nucleus pulposus site, and which is used without bringing a crosslinking agent into contact with the composition.
[54] A method for evaluating a composition applied to the intervertebral disc of a severe intervertebral disc degeneration sheep model, in terms of the ability of the composition to regenerate the intervertebral disc, said method comprising a step of administering a cell-containing composition to a severe intervertebral disc degeneration model produced according to a method comprising the following step (a1) or (a2) and the following step (b1) or (b2):
   (a1) a step of removing nucleus pulposus tissues in an amount corresponding to 0.00004% to 0.00005% of the sheep body weight from a sheep intervertebral disc, so as to produce a degenerated intervertebral disc, or
   (a2) a step of removing 20 mg of nucleus pulposus tissues from a sheep intervertebral disc to produce degenerated intervertebral disc; and
   (b1) a step of further removing nucleus pulposus tissues in an amount corresponding to 0.00014% to 0.000175% of the sheep body weight 4 weeks after the step (a1) or (a2), or
   (b2) a step of further removing 70 mg of nucleus pulposus tissues 4 weeks after the step (a1) or (a2).
[55] The composition according to [54], wherein the step of administering the composition is carried out after the step (b1) or (b2).
[56] The method according to or [55], wherein the evaluation is carried out on a vertebral body and an intervertebral disc collected from a degeneration model after administration of the composition according to at least one evaluation method selected from the group consisting of MRI, histological staining, and immunohistochemical staining (IHC).

Moreover, the present invention includes the following aspects.
[57] A method for the treatment, prevention, or recurrence suppression of intervertebral disc degeneration and/or intervertebral disc damage, wherein
   said method comprises applying a composition comprising a monovalent metal salt of alginic acid and mesenchymal stem cells to the nucleus pulposus site of the intervertebral disc of a subject that is in need of the treatment, prevention, or recurrence suppression.
[58] A method for the treatment, prevention, or recurrence suppression of intervertebral disc degeneration and/or intervertebral disc damage, wherein
   said method comprises applying human bone marrow-derived high-purity mesenchymal stem cells to the nucleus pulposus site of the intervertebral disc of a subject that is in need of the treatment, prevention, or recurrence suppression.
[59] A method for the treatment, prevention, or recurrence suppression of intervertebral disc degeneration and/or intervertebral disc damage, wherein
   said method comprises applying a composition comprising human bone marrow-derived high-purity mesenchymal stem cells and a monovalent metal salt of alginic acid to the nucleus pulposus site of the intervertebral disc of a subject that is in need of the treatment, prevention, or recurrence suppression, and the composition has fluidity upon the application thereof to the nucleus pulposus site.
[60] A method for the treatment, prevention, or recurrence suppression of intervertebral disc pain, wherein
   said method comprises applying a composition comprising human bone marrow-derived high-purity mesenchymal stem cells and a monovalent metal salt of alginic acid to the nucleus pulposus site of the intervertebral disc of a subject that is in need of the treatment, prevention, or recurrence suppression, and the composition has fluidity upon the application thereof to the nucleus pulposus site.
[61] A monovalent metal salt of alginic acid and mesenchymal stem cells, which are for use in the treatment, prevention, or recurrence suppression of intervertebral disc degeneration and/or intervertebral disc damage, in which a composition comprising a monovalent metal salt of alginic acid and mesenchymal stem cells is applied to the nucleus pulposus site of the intervertebral disc of a subject that is in need of the treatment, prevention, or recurrence suppression of intervertebral disc degeneration and/or intervertebral disc damage.
[62] A composition comprising a monovalent metal salt of alginic acid and mesenchymal stem cells, which is for use in the treatment, prevention, or recurrence suppression of intervertebral disc degeneration and/or intervertebral disc damage.
[63] Human bone marrow-derived high-purity mesenchymal stem cells, which are for use in the treatment, prevention, or recurrence suppression of intervertebral disc degeneration and/or intervertebral disc damage.
[64] A composition comprising human bone marrow-derived high-purity mesenchymal stem cells and a monovalent metal salt of alginic acid, which is for use in the treatment, prevention, or recurrence suppression of intervertebral disc degeneration and/or intervertebral disc damage, wherein said composition is applied to the nucleus pulposus site of the intervertebral disc of a subject that is in need of the treatment, prevention, or recurrence suppression, and the composition has fluidity upon the application thereof to the nucleus pulposus site.
[65] A composition comprising human bone marrow-derived high-purity mesenchymal stem cells and a monovalent metal salt of alginic acid, which is for use in the treatment, prevention, or recurrence suppression of intervertebral disc pain, wherein said composition is applied to the nucleus pulposus site of the intervertebral disc of a subject that is in need of the treatment, prevention, or recurrence suppression, and the composition has fluidity upon the application thereof to the nucleus pulposus site.

### Advantageous Effects of Invention

According to the present invention, provided is a composition for filling the nucleus pulposus, which is capable of promoting regeneration of the nucleus pulposus of an intervertebral disc. According to the composition of the present invention, it becomes possible to suppress, not only degenerative change of the nucleus pulposus of an intervertebral disc, but also degenerative changes of the entire intervertebral disc tissues including annulus fibrosis. Moreover, the composition of the present invention has the effect of increasing the ratio of type II collagen-positive hyaline cartilage-like cells in the nucleus pulposus. Furthermore, the composition of the present invention can provide the effect of promoting regeneration, even on cases involving weak self-healing action in the middle and old age, for example, on combined lumbar spinal stenosis (complication of spinal canal stenosis with intervertebral disc herniation) that is frequently observed in middle-aged and older people aged 50 and over. Further, in one aspect of the present invention, taking into consideration the ease of administration of the composition and a reduction in invasiveness to the body, the composition of the present invention is used without treatments of curing a carrier for embedding cells, so that pain associated with intervertebral disc disease, in particular, low back pain, and more preferably, chronic low back pain can be suppressed.

### BRIEF DESCRIPTION OF DRAWINGS

FIG. 1 is a view showing cell sorting data for separation of bone marrow-derived mesenchymal stem cells (BMSCs) labeled with carboxyfluorescein diacetate succinimidyl ester (CFDA-SE) and non-labeled nucleus pulposus cells (NPCs), obtained after co-culture for 7 days using a sodium alginate solution (hereinafter also referred to as “UPAL” (low endotoxin ultra-purified alginate gel).
(a): Two-dimensional (2D) dot plotting of co-cultured cells. P1 gate was disposed around single living cells.
(b): 2D dot plotting of classified non-labeled NPCs in the P2 gate and CFDA-SE-labeled BMSCs in the P3 gate.
FSC-A: forward scattering area; FSC-W: forward scattering width; SSC-A: side scattering area; and CFDA-SE-A: CFDA-SE-area.
   The gene expression of individual cells was plotted on a logarithmic scale (y-axis) with respect to the housekeeping gene GAPDH. Data are shown with an average value of four different rabbit NPC lines.
(c): HIF-1α, (d): GLUT-1, (e): Brachyury, (f): CDMP-1, (g): TGF-β, (h): IGF-1, (i): type II collagen, and (j): aggrecan.
   The data are shown in the form of a mean value±standard error, and the p value was obtained according to one-way ANOVA (analysis of variance) using a Tukey-Kramer post-test.

FIG. 2 is a view showing that bone marrow-derived mesenchymal stem cells (BMSCs) survive in an intervertebral disc (IVD).
Mesenchymal stem cells (BMSCs) derived from a transplanted bone labeled with carboxyfluorescein diacetate succinimidyl ester (CFDA-SE) survive in the intervertebral disc (IVD) 4 weeks and 12 weeks after the surgery. A frozen section of IVD was stained with 4′,6-diamino-2-phenylindole (DAPI). The images are representative images of 4 repeated tests (intact control and BMSCs+Gel, n=4; 4 weeks and 12 weeks). The scale bar=50 lam.

FIG. 3 shows that gels combined with bone marrow-derived mesenchymal stem cells (BMSCs) preserve the water content of degenerated intervertebral discs (IVDs) after discectomy.
(a): T2-weighted, mid-sagittal section images of degenerated IVDs at 4 weeks and 12 weeks after surgery. The images are representative images of eight imaging sessions.
(b): Pfirrmann classification of IVD degeneration. The data are shown in the form of a mean value±standard error (intact control, puncture, discectomy, gel, and BMSCs+gel, n=8; 4 weeks and 12 weeks).
(c) Magnetic resonance imaging (MRI) index (nucleus pulposus (NP) area×mean signal intensity) to degenerative changes in NP. The values are expressed as percentage (%) compared with the intact control IVD. The data are shown in the form of a mean value±standard error, and the p-value was obtained according to one-way ANOVA (analysis of variance) attended with post hoc analysis using the Tukey-Kramer test.

FIG. 4 is a view showing that gel combined with bone marrow-derived mesenchymal stem cells (BMSCs) prevents intervertebral disc (IVD) degeneration after discectomy.
(a, b): Mid-sagittal section images of IVDs stained with hematoxylin and eosin (H & E) or safranin O (representative examples of eight experimental images) (intact control, puncture, discectomy, gel, BMSCs+gel, n=8; 4 weeks and 12 weeks).
AF: Annulus fibrosis, NP: nucleus pulposus. The scale bar=(A): 500 lam (1st and 3rd sections from the top), 50 mm (2nd and 4th sections from the top), and (B): 500 lam.
(c): Histological degeneration grades at 4 weeks and 12 weeks.
   The data are shown in the form of a mean value±standard error, and the p-value was obtained according to one-way ANOVA (analysis of variance) using the Tukey-Kramer post-test.

FIG. 5 is a view showing type II collagen positive cells in rabbit nucleus pulposus (NP).
(a): A mid-sagittal section of a rabbit intervertebral disc (IVD) was stained for type II collagen. The images are representative images of the results of 8 repeated experiments (intact control, puncture, discectomy, gel, BMSCs+gel, n=8; 4 weeks and 12 weeks). The arrow indicates cells positive to type II collagen. The scale bar=500 lam (1st and 3rd sections from the top) and 20 lam (2nd and 4th sections from the top).
(b): The percentage of type II collagen positive cells. The data are shown in the form of a mean value±standard error, and the p-value was obtained according to one-way ANOVA (analysis of variance) using the Tukey-Kramer post-test.

FIG. 6 shows a view showing nucleus pulposus (NP) marker positive cells in rabbit NP.
(a-c): Horizontal sections of rabbit intervertebral discs (IVDs) stained with HIF-1α, GLUT-1 and Brachyury on days 1, 7 and 28. The images are representative images of 4 repeated experiments (intact control, discectomy, and BMSCs+gel, n=4; days 1, 7 and 28). The scale bar=50 lam.
(d-f): The percentage of NP marker positive cells relative to all cells.
(g-i): The percentage of NP marker positive cells to CFDA-SE positive cells (representing transplanted BMSCs). The data are shown in the form of a mean value±standard error, and the p-value was determined using a corresponding t-test.

FIG. 7 is a view showing a mechanism of regeneration of an intervertebral disc (IVD).
Transplanted bone marrow-derived mesenchymal stem cells (BMSCs) produce growth factors and extracellular matrixes (ECMs) that activate existing nucleus pulposus cells (NPCs). Also, activation of the existing NPCs also increases the production of growth factors and ECMs. Transplanted BMSCs differentiate into NPCs. UPAL: ultra-purified alginate (also referred to as “low endotoxin high purity alginate”).

FIG. 8 is a view showing the results of a comprehensive analysis of REC clones.

FIG. 9 is a view showing the expression levels of various types of genes in human healthy intervertebral disc nucleus pulposus cells (NPCs) and high purity mesenchymal cells (RECs).

FIG. 10 is a view showing the results of the MRI of an intervertebral disc in a sheep model 4 weeks after transplantation.

FIG. 11 is a view showing the results of the histological test of an intervertebral disc in a sheep model 4 weeks after transplantation.

FIG. 12 is a view showing the results of the histological test of an intervertebral disc in a sheep model 4 weeks after transplantation.

FIG. 13 is a view showing the expression profiles of human NPCs and RECs at 7 days after 3D co-culture.
(A) An outline view showing REC isolation. The flow cytometric profiles of human bone marrow cells stained for CD271 (LNGFR) and CD90 (THY-1). Cells collected from one well are seeded on a 35-mm culture dishes and are allowed to proliferate until 14 days, so as to obtain a uniform cell population with high differentiation ability and proliferation ability.
(B and C) Using cell sorting data, CFDA-SE-labeled RECs are distinguished from non-labeled NPCs.
(B) The P1 gate eliminates dead cells and debris live cells.
(C) 2D dot plotting showing unlabelled NPCs at the P2 gate and CFDA-SE-labelled RECs at the P3 gate.
(D to K) The gene expression levels of individual cells were normalized against the expression level of the housekeeping gene GAPDH and were plotted on a log scale (y-axis). Data obtained from 4 different human NPC lines were averaged.
(D) HIF-1α, (E) GLUT-1, (F) brachyury, (G) CDMP-1, (H) TGF-β, (I) IGF-1, (J) type II collagen, and (K) aggrecan
   The data are shown in the form of a mean±SD value (n=4). The significant difference was evaluated according to one-way ANOVA (analysis of variance) with a post hoc Tukey-Kramer test.
   NPC: Nucleus pulposus cells
   REC: Rapidly expanding clones
   CFDA-SE: 5,6-Carboxyfluorescein diacetate succinimidyl ester
   ANOVA: Analysis of variance
   FSC-A: Forward scattering area
   FSC-W: Forward scattering width
   SSC-A: Side scattering area
   CFDA-SE-A: CFDA-SE area

FIG. 14 is a view showing the elasticity ratio of two types of gels.
(A) Formation of disk-shaped UPAL and RECs+UPAL gels after gelling (diameter: 4.5 mm; thickness: 2 mm) induced by CaCl₂.
(B and C) A tensile and compression mechanical test apparatus. Samples were compressed at a constant rate of 0.5 mm/min.
(D) Stress-strain curves. Young's modulus was calculated according to the inclination of the tangent line between compression values of 10% to 20%. Four repeated tests were carried out, and representative images are shown.
(E) Young's moduli of the two types of gels. The data indicate a mean±SD value (n=4). The Significant difference was evaluated by a Student's t-test after a Welch's test.
   N.S: No significant difference
   UPAL: Ultra-purified alginate (also referred to as “low endotoxin high purity alginate”)
   RECs: Rapidly expanding clones
   SD: Standard deviation

FIG. 15 is a view showing details of experimental schedule and treatment for each group.
(A and B) 20 mg of Fresh NP tissues were removed from the treated IVD at the first surgery, and a severely degenerated IVD model was established.
(C) 70 mg of Degenerated NP tissues were further removed from the degenerated IVD at 4 weeks after the first surgery.
(D) After the removal of the degenerated NP, a UPAL or RECs+UPAL solution was transplanted into the cavity in the intervertebral disc.
   NP: Nucleus pulposus
   UPAL: Ultra-pure alginic acid (also referred to as “low endotoxin high purity alginic acid”)
   IVD: Intervertebral disc

FIG. 16 is a view showing MRI evaluation of treated IVD at 4 weeks and 24 weeks after the embedding.
(A) T2-weighted, mid-sagittal section images of IVD at 4 weeks and 24 weeks after the surgery in sheep. The mages are representative images of the results of 6 or 8 repeated tests.
(B) IVD degeneration according to Pfirrmann grading.
(C) MRI index (NP area×mean signal intensity) values for degenerative changes in NP. The numerical value is expressed as a percentage (%) of the value to the intact control IVD.
(D) Intervertebral disc height index for IVD treatment. The numerical value is expressed as a percentage (%) of the value to the numerical value of the intact control IVD. The data are shown in the form of a mean±SD value (intact control, n=6; discectomy, n=6; gel, n=8; RECs+gel, n=8). The significant difference was evaluated according to one-way ANOVA with a post hoc analysis using the Tukey-Kramer test.
   NP: Nucleus pulposus
   MRI: Magnetic resonance imaging
   IVD: Intervertebral disc
   ANOVA: Analysis of variance
   SD: Standard deviation

FIG. 17 is a view showing the results obtained by performing histological evaluation of the treated IVD at 4 weeks and 24 weeks after the embedding.
(A and B) Representative mid-sagittal sections of treated IVDs stained with H & E or safranin-O (intact control, n=6; discectomy, n=6; gel, n=8; RECs+gel, n=8). Scale bar—(A): 50 lam (second and fourth sections from top) or 1 mm (first and third sections from top); (B): 1 mm.
(C) Histological grades were determined via modified Boos classification. The data are shown in the form of a mean value±standard deviation. The significant difference was evaluated according to one-way ANOVA (analysis of variance) with a post hoc Tukey-Kramer test.
   IVD: Intervertebral disc
   H & E: Hematoxylin & eosin
   SD: Standard deviation
   AF: Annulus fibrosis
   NP: Nucleus pulposus

FIG. 18 is a view showing type II or type I collagen positive cells in treated IVDs at 4 weeks and 24 weeks after the embedding.
(A and B) Representative mid-sagittal sections of treated IVDs stained for type II collagen or type I collagen (intact control, n=6; discectomy, n=6; gel, n=8; RECs+gel, n=8). Scale bar: 1 mm (first and third lines) or 50 lam (second and fourth lines).
(C and D) The percentage of type II or type I collagen positive cells to all cells in treated IVDs. The data are shown in the form of a mean value±standard deviation. The significant difference was evaluated according to one-way ANOVA (analysis of variance) with a post hoc Tukey-Kramer test.
   IVD: Intervertebral disc
   REC: Rapidly expanding clones
   SD: Standard deviation
   ANOVA: Analysis of variance

FIG. 19 is a view showing histological evaluation at 4 weeks after the removal of NP tissues.
(A) Mid-sagittal sections (stained with H & E or safranin-O) of NP tissues (intact control, or 20, 50, 100 or 200 mg of NP tissues removed) removed from IVDs. In sheep models, IVD degeneration occurred at 4 weeks after the removal of 20 mg or more of the NP tissues. Scale bar: 1 mm (all).
(B) Histological grades were determined via modified Boos classification. The data are shown in the form of a mean value±standard deviation.
   NP: Nucleus pulposus
   IVD: Intervertebral disc
   H & E: Hematoxylin and eosin
   SD: Standard deviation

FIG. 20 is a view showing the results obtained by measuring the height of an intervertebral disc with respect to the height of the intervertebral disc of a vertebra adjacent thereto, using T2-weighted, mid-sagittal images.
“BC” and “EF” mean the heights of forward and backward intervertebral discs, respectively, and “AB” and “DE” mean the heights of the adjacent vertebral bodies on the head side. The DHI value was calculated to be the ratio between the heights of the intervertebral discs (BC+EF) and the heights of the vertebral bodies (AB+DE), and the relative DHI value was calculated to be the ratio of the DHI of treated IVDs to the DHI of intact control IVD.
   DHI: Intervertebral disc height index
   IVD: Intervertebral disc

### DESCRIPTION OF EMBODIMENTS

### 1. Outline

The present invention relates to a composition for regeneration of an intervertebral disc, comprising a monovalent metal salt of alginic acid and mesenchymal stem cells (for example, human bone marrow-derived high-purity mesenchymal stem cells). In one aspect of the present invention, the present invention relates to a composition for regeneration of an intervertebral disc, said composition comprising a low endotoxin monovalent metal salt of alginic acid and mesenchymal stem cells (for example, human bone marrow-derived high-purity mesenchymal stem cells). The composition of the present invention promotes regeneration of the nucleus pulposus of an intervertebral disc via activation of nucleus pulposus cells by mesenchymal stem cells and/or differentiation of mesenchymal stem cells into nucleus pulposus cells. The composition of the present invention is used, such that the composition is applied to the nucleus pulposus site of a subject, and a part of the composition is cured after the application. In several aspects of the present invention, the composition of the present invention is used, such that the composition is applied to the nucleus pulposus site of a subject, and a crosslinking agent is then brought into contact with at least a part of the surface of the composition.

In the present invention, the combined use of bone marrow-derived mesenchymal stem cells (BMSCs) with gel significantly promoted tissue repair effects, compared with a single use of gel, as demonstrated in an in vivo test using mesenchymal stem cells derived from rabbits. With regard to the mechanism thereof, it was elucidated that the co-culture of BMSCs embedded in gel and nucleus pulposus cells provides: 1) interactive activation of the above two types of cells by growth factor production; 2) differentiation of the BMSCs into nucleus pulposus cells; and 3) the improvement of extracellular matrix production ability from the above two types of cells.

The alginic acid comprised in the composition of the present invention is a polysaccharide extracted from brown algae such as kajime, arame and kelp, and has a property by which the alginic acid is cross-linked and cured when divalent metal ions such as calcium are added thereto. By utilizing this property, it is possible for alginic acid to be cured the surface of the composition, and to retain the composition in the affected area, by bringing the alginic acid into contact with metal ions at the affected area. In the present invention, it was found that when a composition comprising low endotoxin sodium alginate is injected in a sol state into an intervertebral disc nucleus pulposus site and a crosslinking agent is then brought into contact with the injection port to cure a part of the composition, degeneration of the nucleus pulposus can be suppressed, the ratio of type II collagen positive cells that are preferable for regeneration of the nucleus pulposus can be increased, and regeneration of the intervertebral disc nucleus pulposus can be promoted.

In the present invention, by embedding mesenchymal stem cells in monovalent metal salts of alginic acid (for example, low endotoxin alginic acid), the embedded mesenchymal stem cells remain in an affected area, and exhibit effects for a long period of time. That is to say, the embedded high purity mesenchymal stem cells produce growth factors and extracellular matrixes, and activate nucleus pulposus cells. Then, the activated nucleus pulposus cells produce growth factors and extracellular matrixes. As a result, the embedded mesenchymal stem cells differentiate into nucleus pulposus cells, and regeneration of the damaged part is promoted via the interaction between the nucleus pulposus cells and the mesenchymal stem cells. In another aspect of the present invention, the cells can be embedded in biocompatible materials such as hyaluronic acid.

In addition, human bone marrow-derived high-purity mesenchymal stem cells (RECs) that are one aspect of mesenchymal stem cells comprised in the composition of the present invention are ultra-high purity human mesenchymal stem cells produced by a technique of directly separating the cells from the bone marrow using two types of antibodies and a cell sorter. Since a uniform cell population can be obtained by separation using a cell sorter, quality control is easy, and the cells have extremely high proliferation ability. Accordingly, it is possible to make a formulation from the cells by mass culture.

The present invention also relates to a composition for regeneration of an intervertebral disc, said composition comprising human bone marrow-derived high-purity mesenchymal stem cells. In one aspect of the present invention, the composition of the present invention is applied to the nucleus pulposus of an intervertebral disc in an undifferentiated state and/or without treatments of induction of differentiation. The composition of the present invention promotes regeneration of the nucleus pulposus of an intervertebral disc via activation of nucleus pulposus cells by human bone marrow-derived high-purity mesenchymal stem cells and/or differentiation of mesenchymal stem cells into nucleus pulposus cells. The composition of the present invention can be used, such that the composition is applied to the nucleus pulposus site of a subject, and a crosslinking agent is brought into contact with at least a part of the surface thereof. In one aspect of the present invention, the composition of the present invention can be used, such that the composition is applied to the nucleus pulposus site of a subject, and a part of the composition is cured after application.

### 2. Definition

The terms “low endotoxin” and “monovalent metal salt of alginic acid” will be described later.

The “intervertebral disc” is a columnar tissue lying between vertebrae forming the vertebral column. An intervertebral disc is a disc-shaped avascular tissue, and has a structure in which an annulus fibrosus surrounds a nucleus pulposus at the center and endplates are disposed above and below.

The “nucleus pulposus” is a gel-like tissue located at the center of an intervertebral disc, which mainly contains nucleus pulposus cells, an extracellular matrix mainly composed of proteoglycan and Type II collagen, and water. The nucleus pulposus is considered to have extremely low self-repairing and regenerating capacity.

“Filling of nucleus pulposus” refers to filling of a degenerated part, a shrunken part, or a removed part of a degenerated, shrunken, or removed nucleus pulposus resulting from aging, trauma, infection, a surgical operation therefor (for example, an intervertebral discectomy (resection)), or the like. The term “filling of nucleus pulposus” in the present specification is used with the same meaning as “nucleus pulposus replenishment”, and a “composition for replenishing a nucleus pulposus” is synonymous with a “composition for filling a nucleus pulposus”.

The “nucleus pulposus site” refers to a site where a nucleus pulposus is present, a degenerated or shrunken site of a nucleus pulposus, or a defective part of a nucleus pulposus formed by removing at least a part of the nucleus pulposus, and also includes a surrounding part of the site where the nucleus pulposus is present.

The “subject” refers to a human or a living thing other than a human, for example, a bird and a non-human mammal (for example, a cow, monkey, cat, mouse, rat, guinea pig, hamster, pig, dog, rabbit, sheep, and horse).

The “application” means filling of a nucleus pulposus site of an intervertebral disc using the composition of the present invention in an amount sufficient to embed a degenerated part, a shrunken part, a removed part, a defective part, or the like of the nucleus pulposus site.

The verb “to embed” means that the cells are suspended or mixed into a solution of biocompatible materials, and preferably, of monovalent metal salts of alginic acid.

The phrase “partially cured” means as described later.

The phrase “to comprise a low endotoxin monovalent metal salt of alginic acid” means that the composition of the present invention comprises a low endotoxin monovalent metal salt of alginic acid in an amount sufficient for regeneration of the nucleus pulposus in a nucleus pulposus site to which the composition of the present invention is applied.

The phrase “to have fluidity” means as described later.

The phrases “intervertebral disc degeneration and/or intervertebral disc damage” and “treatment, prevention, or recurrence suppression” mean as described later.

The “intervertebral disc pain” means pain caused by the intervertebral disc. The “low back pain” means pain occurring in the site around the intervertebral disc, and includes back pain and/or buttock pain.

The “chronic low back pain” means low back pain that continues for 12 weeks or more after the onset of the low back pain.

The composition of the present invention may be provided in the form of a solution, using a solvent, or may also be provided in a dry state such as a freeze-dried body (in particular, freeze-dried powders). In a case where the composition of the present invention is provided in a dry state, the solvent is used upon the application of the present composition, so that the present composition is used in the state of a composition having fluidity, such as a solution. The solvent used herein is not particularly limited, as long as it is applicable to a living body. Examples of such a solvent may include water for injection, purified water, distilled water, ion exchange water (or deionized water), Milli Q water, a normal saline, and a phosphate-buffered saline (PBS). The solvent is preferably water for injection, distilled water, a normal saline or the like, which can be used for the treatment of humans and animals.

### 3. Monovalent Metal Salt of Alginic Acid

In the present invention, the “monovalent metal salt of alginic acid” is a water-soluble salt formed by ion exchange between a hydrogen atom of carboxylic acid at position 6 of alginic acid and a monovalent metal ion such as Na+ or K+. Although specific examples of monovalent metal salts of alginic acid include sodium alginate and potassium alginate, sodium alginate acquirable as a commercially available product is particularly preferable. A solution of a monovalent metal salt of alginic acid forms a gel when mixed with a crosslinking agent.

The “alginic acid” used in the present invention is a biodegradable, high molecular weight polysaccharide that is a polymer obtained by linearly polymerizing two types of uronic acids in the form of D-mannuronic acid (M) and L-gluronic acid (G). More specifically, the alginic acid is a block copolymer in which a homopolymer fraction of D-mannuronic acid (MM fraction), homopolymer fraction of L-gluronic acid (GG fraction) and fraction in which D-mannuronic acid and L-gluronic acid are randomly arranged (MG fraction) are linked arbitrarily. The composite ratio of the D-mannuronic acid to the L-gluronic acid of the alginic acid (M/G ratio) mainly varies according to the type of algae or other organism serving as the origin thereof, is affected by the habitat and season of that organism, and extends over a wide range from a high G type having an M/G ratio of about 0.4 to a high M type having an M/G ratio of about 5.

While a monovalent metal salt of alginic acid is a high molecular weight polysaccharide and it is difficult to accurately determine the molecular weight thereof, it has a weight-average molecular weight generally in a range of 10,000 to 10,000,000, preferably 20,000 to 8,000,000 and more preferably 50,000 to 5,000,000 since too low molecular weight results in low viscosity, by which adhesion to the tissue surrounding the applied site may become weak and too high molecular weight makes the production difficult, lowers solubility, makes handling poor due to too high viscosity in the solution state, makes it difficult to maintain the physical properties during long-term preservation, and the like. In the present specification, numerical ranges expressed with “to” each represent a range that includes the numerical values preceding and following “to” as minimum and maximum values, respectively.

Meanwhile, differences in values according to the measurement method are known to occur in the measurement of molecular weights of high molecular weight substances derived from a natural origin. For example, a weight-average molecular weight measured by gel permeation chromatography (GPC) or gel filtration chromatography (which are also collectively referred to as size exclusion chromatography) is preferably 100,000 or more and more preferably 500,000 or more, while preferably 5,000,000 or less and more preferably 3,000,000 or less. The preferable range is 100,000 to 5,000,000, and more preferably 500,000 to 3,500,000.

Furthermore, an absolute weight-average molecular weight can be measured, for example, by a GPC-MALS method employing a combination of gel permeation chromatography (GPC) and a multi-angle light scattering detector (Multi Angle Light Scattering: MALS). The weight-average molecular weight (absolute molecular weight) measured by the GPC-MALS method is, according to the effects shown in the examples of the present invention, preferably 10,000 or more, more preferably 80,000 or more, and still more preferably 90,000 or more, while preferably 1,000,000 or less, more preferably 800,000 or less, still more preferably 700,000 or less, and particularly preferably 500,000 or less. The preferable range is 10,000 to 1,000,000, more preferably 80,000 to 800,000, still more preferably 90,000 to 700,000, and particularly preferably 90,000 to 500,000.

When a molecular weight of a high molecular weight polysaccharide is calculated by the process described above, usually, there is normally the potential for measurement error of 10 to 20% or more. For example, a molecular weight of 400,000 can fluctuate within the range of 320,000 to 480,000, a molecular weight of 500,000 can fluctuate within the range of 400,000 to 600,000, and a molecular weight of 1,000,000 can fluctuate within the range of 800,000 to 1,200,000.

A molecular weight of a monovalent metal salt of alginic acid can be measured according to a common method.

Typical conditions for molecular weight measurement using gel permeation chromatography are as described in the examples of the present specification. For example, GMPW-XL×2+G2500PW-XL (7.8 mm I.D.×300 mm) may be used as the columns, a 200 mM aqueous sodium nitrate solution can be used as the eluent, and pullulan can be used as the molecular weight standard.

Typical conditions for molecular weight measurement using GPC-MALS are as described in the examples of the present specification. For example, an RI detector and a light scattering detector (MALS) can be used as the detectors.

Although a monovalent metal salt of alginic acid has a large molecular weight and relatively high viscosity when originally extracted from brown algae, the molecular weight becomes smaller and the viscosity becomes lower during the course of heat drying, purification and the like. Through management of the conditions such as the temperature during the production, selection of brown alga used for the raw material, processes like molecular weight fractionation during the production, and the like, monovalent metal salts of alginic acid with different molecular weights can be produced. Furthermore, it can be mixed with a monovalent metal salt of alginic acid from other lot having different molecular weight or viscosity, so as to give a monovalent metal salt of alginic acid having a molecular weight of interest.

A monovalent metal salt of alginic acid used with the present invention is preferably a solution obtained by dissolving a monovalent metal salt of alginic acid into MilliQ water to a concentration of 1 w/w %, where the apparent viscosity as measured with a cone-plate viscometer under the condition of 20° C. is preferably 40 mPa·s to 800 mPa·s and more preferably 50 mPa·s to 600 mPa·s. The conditions for measuring the apparent viscosity preferably follow the conditions described hereinbelow. An “apparent viscosity” in the present specification may simply be referred to as “viscosity”.

Although the alginic acid used in the present invention may be of a natural origin or synthetic, it is preferably derived from a natural origin. Examples of naturally-occurring alginic acids include those extracted from brown algae. Although brown algae containing alginic acid are prominently found along seacoasts throughout the world, algae that can actually be used as raw materials of alginic acid are limited, with typical examples thereof including Lessonia found in South America, Macrocystis found in North America, Laminaria and Ascophyllum found in Europe, and Durvillea found in Australia. Examples of brown algae serving as raw materials of alginic acid include genus Lessonia, genus Macrocystis, genus Laminaria (Laminariaceae), genus Ascophyllum, genus Durvillea, genus Eisenia and genus Ecklonia.

In another aspect of the present invention, as carriers for embedding cells, other biocompatible materials may also be used. For instance, one or more, natural or synthetic glycosaminoglycans (GAG) or mucopolysaccharides, such as hyaluronic acid (HA), chondroitin sulfate, dermatan sulfate, keratin sulfate, heparin, heparan sulfate, galactosaminoglycuronglycan sulfate (GGGS), and a pharmaceutically acceptable salt thereof (a physiological salt), may be used as a carrier(s) for embedding cells. In another aspect of the present invention, as a carrier(s) for embedding cells, any one or more selected from among polysaccharides such as cellulose, a cellulose derivative, agarose, chitin, chitosan, starch and pectin, proteins such as gelatin, collagen and polypeptide, amino acid derivatives, copolymers thereof, and derivatives thereof, may be used, although the examples of the carrier(s) are not particularly limited thereto. In another aspect of the present invention, hydrogel such as a collagen- or gelatin-like composition may also be used as a carrier.

### 4. Endotoxin Reduction Treatment

The monovalent metal salt of alginic acid used in the present invention is not particularly limited, and it is, for example, a monovalent metal salt of alginic acid with low endotoxin. In one aspect of the present invention, the carrier for embedding cells preferably has low endotoxin. That is, the monovalent metal salt of alginic acid used in the present invention is preferably a low endotoxin monovalent metal salt of alginic acid. The term “low endotoxin” is used herein to mean that the endotoxin level is low to such an extent that it substantially does not provoke inflammation or fever. More preferably, the monovalent metal salt of alginic acid used in the present invention is desirably a monovalent metal salt of alginic acid that is subjected to an endotoxin reduction treatment.

The endotoxin reduction treatment can be performed by a known method or a method complying therewith. For example, this treatment can be carried out by the method of Suga et al. involving purification of sodium hyaluronate (refer to, for example, Japanese Patent Application Laid-open No. H9-324001), the method of Yoshida et al. involving purification of β1,3-glucan (refer to, for example, Japanese Patent Application Laid-open No. H8-269102), the method of William et al. involving purification of a biopolymer such as alginate or gellan gum (refer to, for example, Japanese Translation of PCT Application No. 2002-530440), the method of James et al. involving purification of polysaccharide (refer to, for example, WO 93/13136), the method of Lewis et al. (refer to, for example, U.S. Pat. No. 5,589,591), the method of Hermanfranck et al. involving purification of alginate (refer to, for example, Appl. Microbiol. Biotechnol. (1994), 40:638-643) or a method complying therewith. The endotoxin reduction treatment is not limited thereto, but rather can be carried out by a known method such as cleaning, purification using filtration with filter (endotoxin removing filter or electrification filter), ultrafiltration or a column (such as an endotoxin adsorption affinity column, gel filtration column or ion exchange column), adsorption to a hydrophobic substance, resin or activated carbon and the like, organic solvent treatment (such as extraction with an organic solvent or precipitation or deposition by addition of organic solvent), surfactant treatment (refer to, for example, Japanese Patent Application Laid-open No. 2005-036036), or a suitable combination thereof. A known method such as centrifugal separation may be suitably combined with these treatment steps. It is desirable that a method is suitably selected according to the type of alginic acid.

The endotoxin level can be confirmed by a known method, and can be measured using a known method such as a method using Limulus reagent (LAL) or Endospecy (registered trademark) ES-24S set (Seikagaku Corporation).

Although there are no particular limitations on the endotoxin treatment method of the monovalent metal salt of alginic acid contained in the composition of the present invention, the endotoxin content of the monovalent metal salt of alginic acid in the case of measuring endotoxin using a limulus reagent (LAL) is preferably 500 endotoxin units (EU)/g or less, more preferably 100 EU/g or less, even more preferably 50 EU/g or less, and particularly preferably 30 EU/g or less as a result thereof. Sodium alginate that has undergone the endotoxin reduction treatment can be acquired as a commercially available products such as Sea Matrix (registered trademark) (Mochida Pharmaceutical), PRONOVA™ UP LVG (FMC BioPolymer) or the like.

Sodium alginate that has been subjected to an endotoxin reduction treatment as described above is also referred to as “ultra-purified sodium alginate (UPAL)” in the present specification.

### 5. Preparation of Solution of Monovalent Metal Salt of Alginic Acid

The composition of the present invention may be prepared by using a solution of a monovalent metal salt of alginic acid. The solution of a monovalent metal salt of alginic acid can be prepared by a known method or method complying therewith. Namely, the monovalent metal salt of alginic acid used in the present invention can be produced by a known method such as an acid method or calcium method using the previously described brown algae. More specifically, after extracting from these brown algae using an alkaline aqueous solution such as aqueous sodium carbonate solution, for example, alginic acid be obtained by adding an acid (such as hydrochloric acid or sulfuric acid), and a salt of alginic acid can be obtained by ion exchange of the alginic acid. Preferably, the endotoxin reduction treatment may be performed as previously described. There are no particular limitations on the solvent of the monovalent metal salt of alginic acid provided it is a solvent that can be applied to a biological body, and examples of such solvents include purified water, distilled water, ion exchange water, Milli-Q water, physiological saline and phosphate-buffered saline (PBS). These are preferably sterilized and preferably subjected to endotoxin reduction treatment. For example, Milli-Q water can be used after sterilizing by filtration.

When the composition of the present invention is provided in a dry state as a lyophilizate or the like, the above-described solvent can be used to prepare it into a solution having fluidity.

Moreover, all of the operations for obtaining the composition of the present invention are preferably carried out in an environment at a low endotoxin level and a low bacterial level. For example, the operations are preferably carried out in a clean bench using sterilized tools. The tools used may be treated with a commercially available endotoxin removal agent.

### 6. Apparent Viscosity of Composition of Present Invention

The composition of the present invention in some aspects is in a liquid state having fluidity, namely, a solution state. The composition of the present invention has fluidity when applied to the nucleus pulposus site (for example, the nucleus pulposus cavity part). In one aspect of the present invention, the composition of the present invention preferably has fluidity that allows injection with a 21 G needle following an hour of standing at 20° C. While the apparent viscosity of the composition of the present invention in this aspect is not particularly limited as long as the effect of the present invention can be achieved, it is preferably 10 mPa·s or more, more preferably 100 mPa·s or more, still more preferably 200 mPa·s or more, and particularly preferably 500 mPa·s or more since too low viscosity would weaken adhesion to the tissue surrounding the applied site. It is also preferably 50,000 mPa·s or less, more preferably 20,000 mPa·s or less, and still more preferably 10,000 mPa·s or less since too high apparent viscosity would deteriorate the handling property. An apparent viscosity of 20,000 mPa·s or less would facilitate application with a syringe or the like. Application, however, is also possible even if the apparent viscosity is 20,000 mPa·s or more by using a pressurized or electric filling tool or other means. The composition of the present invention is preferably in a range of 10 mPa·s to 50,000 mPa·s, more preferably 100 mPa·s to 30,000 mPa·s, still more preferably 200 mPa·s to 20,000 mPa·s, yet still more preferably 500 mPa·s to 20,000 mPa·s, and particularly preferably 700 mPa·s to 20,000 mPa·s. In another preferable aspect, it may be 500 mPa·s to 10,000 mPa·s, or 2,000 mPa·s to 10,000 mPa·s. The composition of the present invention in some aspects has viscosity that also allows application to a subject with a syringe or the like.

The apparent viscosity of a composition containing a monovalent metal salt of alginic acid, for example, an aqueous solution of alginic acid, can be measured according to a common method. For example, a coaxial double cylinder type rotational viscometer, a single cylinder type rotational viscometer (Brookfield viscometer), a cone-plate rotational viscometer (a cone-plate viscometer), or the like can be used for the measurement according to a rotational viscometer method. It is preferable to follow the viscosity measurement method of the Japanese Pharmacopoeia (16th edition). According to the present invention, the viscosity measurement is preferably carried out under the condition of 20° C. As will be described below, if the composition of the present invention contains anything that cannot be dissolved in the solvent such as cells, the apparent viscosity of the composition is preferably an apparent viscosity free of cells or the like in order to carry out an accurate viscosity measurement.

In the present invention, it is desirable that an apparent viscosity of the composition containing a monovalent metal salt of alginic acid is particularly measured using a cone-plate viscometer. For example, a measurement preferably takes place under the following measurement conditions. A sample solution is prepared with MilliQ water. The measurement temperature is 20° C. The rotation speed of the cone-plate viscometer is 1 rpm for measuring a 1% solution of the monovalent metal salt of alginic acid, 0.5 rpm for measuring a 2% solution, which can be determined so on. The reading time is 2 minutes of measurement for the 1% solution of the monovalent metal salt of alginic acid to obtain an average value between 1 minute to 2 minutes after the start. The reading time is 2.5 minutes of measurement for the 2% solution to obtain an average value between 0.5 minutes to 2.5 minutes after the start. The test value is an average value of three times of measurements.

The apparent viscosity of the composition of the present invention can be adjusted, for example, by controlling the concentration, the molecular weight, the M/G ratio or the like of the monovalent metal salt of alginic acid.

The apparent viscosity of the monovalent metal salt solution of alginic acid becomes high when the concentration of the monovalent metal salt of alginic acid in the solution is high whereas the viscosity becomes low when the concentration is low. Moreover, the viscosity becomes higher when the molecular weight of the monovalent metal salt of alginic acid is large whereas the viscosity becomes lower when the molecular weight is small.

Since an apparent viscosity of a monovalent metal salt solution of alginic acid is affected by the M/G ratio, for example, an alginic acid can be suitably selected that has an M/G ratio more preferable for viscosity of the solution or the like. The M/G ratio of the alginic acid used with the present invention is about 0.1 to 5.0, preferably about 0.1 to 4.0, and more preferably about 0.2 to 3.5. As described above, since the M/G ratio is mainly determined by the species of the seaweed, the species of the brown alga used as the raw material affects the viscosity of the monovalent metal salt solution of alginic acid. The alginic acid used with the present invention is preferably derived from a brown alga of genus Lessonia, genus Macrycystis, genus Laminaria, genus Ascophyllum and genus Durvillea, more preferably from a brown alga of genus Lessonia, and particularly preferably derived from Lessonia nigrescens.

### 7. Mesenchymal Stem Cells

The mesenchymal stem cells used in the present invention are somatic stem cells derived from mesodermal tissues (mesenchyme), and are expected to be applied to regenerative medicine, such as reconstruction of bone, blood vessel, and cardiac muscle.

The mesenchymal stem cells can be obtained from various tissues such as bone marrow, adipose tissues, placental tissues, dental pulp, or umbilical cord tissues. The purification process thereof is, for example, as follows.

From a mixed population of cells obtained by enzymatic treatment of small amounts of fat sections collected from humans or non-human mammals (for example, bovines, monkeys, cats, mice, rats, guinea pigs, hamsters, swines, dogs, rabbits, sheep, horses, goats, rabbits, etc.), a floating fat cell population is separated by centrifugation. Thereafter, the floating fat cell population is left at rest in a state in which it is brought into contact with the ceiling of a culture vessel filled with a culture solution. At that time, fibroblast-like cells that are precipitated and grow on the lower bed are allowed to grow by subculture.

Moreover, in the present invention, iPS cell-derived mesenchymal stem cells, or commercially available mesenchymal stem cells can also be used. In the present invention, the mesenchymal stem cells are preferably applied to the nucleus pulposus, in an undifferentiated state and/or without treatments of induction of differentiation. The undifferentiated state means that the stem cells having differentiation ability remain undifferentiated. The term “without treatments of induction of differentiation” means that, for example, stem cells having differentiation ability are not treated, for example, are not differentiated into specific cells using a differentiation induction medium

### 8. Human Bone Marrow-Derived High-Purity Mesenchymal Stem Cells

### (1) REC Clone

In the previous studies, the present inventor had succeeded in isolating rapidly expanding cell clones (Rapidly Expanding Clone: REC) having rapid proliferation ability, from LNGFR (CD271)-positive mesenchymal stem cells (CD271+ cells), or LNGFR (CD271) and Thy-1 (CD90) double-positive mesenchymal stem cells (CD271+CD90+ cells).

These RECs can reach confluence in 2 weeks when they are seeded at a density of a single cell/well on a 96-well plate and are then cultured, and all of the proliferation ability, differentiation ability and migration ability of these cells are 1000-fold higher than those of mesenchymal stem cells obtained by conventional methods. In particular, since RECs retain migration ability, the RECs can be administered via intravenous administration, and it can be expected that the RECs are applied to serious systemic diseases such as osteochondrodysplasia.

In the present invention, among the above-described REC clones, cell clones with less variation in differentiation ability and proliferation ability can be used.

The cell population comprising the cell clones of the present invention is a cell population comprising LNGFR (CD271) and Thy-1 (CD90) double-positive, rapidly proliferating mesenchymal stem cell clones, and said cell population satisfies at least one of the following characteristics (a) and (b):
(a) the coefficient of variation of forward scattered light in flow cytometry is 40% or less; and
(b) the average cell size is 20 nm or less.

### (2) Human Mesenchymal Stem Cell Concentration Method

In the present invention, in order to obtain LNGFR (CD271)-positive, or LNGFR (CD271) and Thy-1 (CD90) double-positive mesenchymal stem cells, for example, the method described in WO2009/31678 can be applied.

An outline of the method is as follows.

First, from a cell population comprising human mesenchymal stem cells, LNGFR (CD271)-positive (CD271+), or CD271 and CD90 double-positive (CD271+CD90+) cell fractions are sorted, so that the mesenchymal stem cells are highly concentrated. Besides, when such a cell population comprising human mesenchymal stem cells comprises hematopoietic cells, in order to sort non-hematopoietic cells, a step of sorting CD45 and CD235a co-negative cells (CD45−CD235a−) may be added.

A cell population comprising mesenchymal stem cells can be prepared according to flow cytometry or affinity chromatography.

The materials for obtaining this cell population are not particularly limited, and examples of the materials may include bone marrow, adipose tissues, cord blood, and peripheral blood (including peripheral blood after G-CSF administration). Besides, the bone marrow from spine, sternum, ilium, etc. may be used as bone marrow herein. In addition, examples of the cells used herein may also include ES cells and iPS cells.

Upon preparation of the cells, if the material becomes a cell mass containing mesenchymal stem cells, a physical treatment involving pipetting, or an enzyme treatment using trypsin, collagenase, etc. can be performed on the material, as necessary. If the material contains erythrocytes, the erythrocytes are preferably lysed in advance.

Using the thus prepared cell population, CD271+ cells, or CD271+CD90+ cells are sorted.

As a method of sorting CD271+ cells or CD271+CD90+ cells, a method using an antibody is applied, for example.

The antibodies to be used herein are an anti-CD271 antibody and/or an anti-CD90 antibody, which are capable of sorting CD271+ cells or CD271+CD90+ cells. In the case of using flow cytometry for sorting, anti-CD271 antibodies labeled with different fluorescent dyes such as FITC, PE and APC, or an anti-CD271 antibody and an anti-CD90 antibody are used in appropriate combination, so that living cells can be sorted in a short time. Moreover, other than such flow cytometry, CD271+CD90+ cells can be sorted by a method using magnetic beads or a method using affinity chromatography.

Besides, before application of these methods, a fluorescent dye (for example, PI) that stains dead cells has previously been reacted with the cell population, and the cells stained with the fluorescent dye have been removed, so that the dead cells may be eliminated.

### (3) REC Cell Concentration

Subsequently, the sorted LNGFR-positive, or LNGFR and Thy1 double-positive cells are subjected to single cell (clone) culture, and rapidly proliferating lots are then selected, so that high purity human mesenchymal stem cells having excellent proliferation ability, differentiation ability and migration ability (Rapidly Expanding Clones: RECs) can be obtained.

Mononuclear cells are prepared from human bone marrow or fat/placental chorionic villi, and bone marrow mononuclear cells are then stained with anti-LNGFR alone, or with anti-LNGFR and anti-Thy1. Thereafter, using flow cytometry (cell sorter), LNGFR-positive cells, or LNGFR-positive and Thy1-positive cells, are clone-sorted onto a 96-well culture plate. That is, the cells are seeded at a cell density of a single cell/well on each well. Two weeks after the single cell culture, the culture plate is photographed under a microscope, and the wells that become confluent or semi-confluent are sorted, and the cells contained in these wells are designated to be RECs.

Herein, the terms “rapidly proliferating” and “rapidly expanding” mean that cells have such a proliferation rate that when the cells are seeded at a cell density of a single cell/well on a 96-well culture plate and are cultured, the cell plate becomes confluent or semi-confluent two weeks after initiation of culture or earlier (doubling time: 26±1 hours).

Confluent is a state in which 90% or more of the surface of a culture vessel (culture surface) is covered with cultured cells. Semi-confluent is a state in which 70% to 90% of the surface of a culture vessel (culture surface) is covered with cultured cells. The size and type of a culture device used can be changed, as appropriate, depending on the proliferation rate of the cells. Slowly proliferating cells (i.e. Moderately/Slowly Expanding Cells), namely, cells that do not become semi-confluent or confluent even 2 weeks after initiation of the single cell culture, are discarded. The RECs recovered from each well and sorted as RECs used herein are transferred into a culture flask for each well, and are further cultured until they become confluent (expansion culture). Thereafter, the expanded cells are recovered, separately. RECs derived from one well are defined to be 1 lot.

Since the RECs used in the present invention are obtained by clone sorting, in which a single cell is seeded on a single well, all of the proliferating cells have the same genetic trait as one another. Accordingly, in the present invention, the entire cell population may be referred to as a “clone”, or individual cells constituting such a cell population may be referred to as a “clone.”

In addition, in the present invention, RECs used in sorting can be evaluated in advance, using a REC marker (anti-Ror2). For example, after the above-described expansion culture, adhering and proliferating cells are recovered from all lots, and some cells (approximately 1 to 3×10⁵ cells) are separated from each lot, and are subjected to single staining with a monoclonal antibody against anti-Ror2. The method of performing single staining with a monoclonal antibody against anti-Ror2 has been known (WO2016/17795). To sum up, according to flow cytometric analysis using a REC marker, the percentage of REC marker-positive cells in the recovered cells is obtained. To obtain the percentage, the expression of Ror2 mRNA may be quantified according to quantitative PCR, or the percentage may also be obtained manually under a microscope. A lot (cell population), in which the above-described positive percentage is a predetermined value (for example, 65%) or more, is determined to be satisfactory, and this lot can be used in the after-mentioned sorting.

### (4) Sorting of Stem Cell Population

In the present invention, with regard to REC clones in each lot, cell proliferation ability, adipose differentiation ability, the expression level of a REC-specific marker, and cell size uniformity are examined, and correlation among them is then analyzed, so that it has become possible to sort high purity RECs having higher cell performance.

In the present invention, the coefficient of variation (CV value) of forward scattered light and the average size of the cells are used as indicators for sorting.

The forward scattered light (Forward Scatter) is a light scattered at a small angle forward to the axis of the laser light. The forward scattered light consists of the scattered light, diffracted light and refracted light of a laser light generated on the surface of a cell, and provides information about the size of a sample.

The coefficient of variation (CV) is a value obtained by dividing a standard deviation by a mean value, and is a numerical value used to relatively evaluate a variation of data with different units and the relationship between data and variation relative to the mean value.

In the present invention, a cell population having the above-described CV value that is 40% or less, and preferably 35% or less, is sorted. The cell population having a CV value that of 40% or less, and preferably 35% or less, is composed of cells having a uniform size. The CV value is preferably 30% or less, 25% or less, or 20% or less. In addition, the average size of cells in the cell population sorted by the present invention is 20 lam or less. The average size of the cells is preferably 18 lam or less, and is in the range of 14 μm to 18 μm.

The present invention also provides a method for evaluating the quality of a cell population of LNGFR-positive, or LNGFR (CD271) and Thy-1 (CD90) double-positive, rapidly proliferating mesenchymal stem cell clones. In the present invention, a cell population satisfying at least one of the following characteristics (a) and (b), and preferably, both of the following characteristics (a) and (b), is determined to have high quality:
(a) the coefficient of variation of forward scattered light in flow cytometry is 40% or less; and
(b) the average cell size is 20 lam or less.

With regard to the thus evaluated and sorted cell population, the number of cell clones constituting the cell population is not limited, and the cell population has, for example, approximately 0.8×10⁷ to 1.2×10⁷ cells in 1 ml of a solution.

Furthermore, in a preferred aspect of the present invention, a cell population satisfying at least one of the following characteristics (a) and (b), and preferably, both of the following characteristics (a) and (b), is determined to have high quality:
(a) the coefficient of variation of forward scattered light in flow cytometry is 35% or less; and
(b) the average cell size is 20 lam or less.

With regard to the thus evaluated and sorted cell population, the number of cell clones constituting the cell population is not limited, and the cell population has, for example, approximately 0.8×10⁷ to 1.2×10⁷ cells in 1 ml of a solution.

### 9. Preparation of Composition of the Present Invention

The composition of the present invention is characterized in that it comprises, for example, a low endotoxin monovalent metal salt of alginic acid and the above-described mesenchymal stem cells as active ingredients. The present inventors have found for the first time that when the composition of the present invention is filled into the nucleus pulposus site of a living body, the monovalent metal salt of alginic acid itself exhibits regenerative or therapeutic effects on the nucleus pulposus tissues. The phrase “to comprise . . . as an active ingredient” may mean that the low endotoxin monovalent metal salt of alginic acid may be comprised in the composition of the present invention in an amount that can exhibit regenerative or therapeutic effects on nucleus pulposus tissues, when the low endotoxin monovalent metal salt of alginic acid is applied to the affected area. The concentration of the low endotoxin monovalent metal salt of alginic acid is, at least, preferably 0.1 w/v % or more, more preferably 0.5 w/v % or more, and further preferably 1 w/v %, with respect to the total concentration of the composition. The preferred concentration of the monovalent metal salt of alginic acid in the composition of the present invention is preferably 0.5 w/v % to 5 w/v %, more preferably 1 w/v % to 5 w/v %, further preferably 1 w/v % to 3 w/v %, and particularly preferably 1.5 w/v % to 2.5 w/v %. In addition, in another aspect, the concentration of the monovalent metal salt of alginic acid in the composition of the present invention may be preferably 0.5 w/w % to 5 w/w %, more preferably 1 w/w % to 5 w/w %, further preferably 1 w/w % to 3 w/w %, and particularly preferably 1.5 w/w % to 2.5 w/w %.

When the composition is produced, as described above, using a monovalent metal salt of alginic acid that is purified to such an extent that it exhibits a preferred endotoxin level, the content of endotoxin in the composition is generally 500 EU/g or less, preferably 300 EU/g or less, more preferably 150 EU/g or less, and particularly preferably 100 EU/g or less.

The number of cells (cell concentration) comprised in the composition of the present invention is, for example, 1×10⁴ cells/ml or more, or 1×10⁵ cells/ml or more, and preferably 1×10⁴ cells/ml to 1×10⁷ cells/ml.

The composition of the present invention may also comprise factors that promote the growth of cells. Examples of such factors may include BMP, FGF, VEGF, HGF, TGF-β, IGF-1, PDGF, CDMP (cartilage-derived-morphogenetic protein), CSF, EPO, IL, PRP (Platelet Rich Plasma), SOX and IF. These factors may be produced according to a recombination method, or may also be purified from protein compositions. Besides, in several aspects of the present invention, the composition does not comprise these growth factors. Even in a case where the composition does not comprise such growth factors, regeneration of the nucleus pulposus is sufficiently carried out, and higher safety is obtained, compared with the case of actively promoting the growth of cells.

The composition of the present invention may also comprise a factor that suppresses cell death. There are factors causing cell death, such as, for example, Caspase and TNFα. Examples of the factor suppressing these factors may include an antibody and siRNA. These cell death-suppressing factors may be produced according to a recombination method, or may also be purified from protein compositions. Besides, in several aspects of the present invention, the composition does not comprise these cell death-suppressing factors. Even in a case where the composition does not comprise such cell death-suppressing factors, regeneration of the nucleus pulposus is sufficiently carried out, and higher safety is obtained, compared with the case of actively suppressing cell death.

Besides, in one aspect of the present invention, the composition of the present invention does not comprise components exhibiting pharmacological action against the nucleus pulposus tissues of an intervertebral disc, other than the low endotoxin monovalent metal salt of alginic acid. Such a composition comprising only the low endotoxin monovalent metal salt of alginic acid as an active ingredient can exhibit sufficient regenerative or therapeutic effects on the nucleus pulposus.

In several aspects of the present invention, the composition of the present invention may also comprise other pharmaceutically active ingredients, and ingredients generally used in medicaments, such as commonly used stabilizers, emulsifiers, osmotic regulators, buffer agents, tonicity agents, preservatives, soothing agents, and coloring agents, as necessary.

### 10. Curing of Composition of the Present Invention

The composition of the present invention is used such that a part of the composition is cured after the application thereof to a nucleus pulposus site.

“Partially cured” means to bring a crosslinking agent into contact with a part of the composition of the present invention having fluidity so as to gel and solidify not the whole but a part of the composition in contact with the crosslinking agent. Preferably, the crosslinking agent is brought into contact with at least a part of the surface of the composition of the present invention having fluidity so as to cure a part of the composition of the present invention. In the present invention, a crosslinking agent and a curing means that are suitable for a carrier used can be selected.

In some aspects, “the composition is partially cured after being applied to the nucleus pulposus site” means that at least 50% of the volume of the composition in a 6 mm diameter test tube is not gelled when the test tube is filled with 500 μL of a sodium alginate and a crosslinking agent by employing the same method and ratio for using the crosslinking agent as those employed for filling in the nucleus pulposus site, and leaving the resultant to stand for an hour in vitro, where the ungelled part may be represented by suction of at least 50% of the volume of the composition in the test tube using a syringe with a 21 G needle.

As long as the composition shows such property after being filled into the nucleus pulposus site, it is considered that the composition would not deviate therefrom even when compression force is applied from the head and tail sides of the intervertebral disc after the filling. “At least a part of the surface of the composition” refers to, for example, an opening in the surface of the intervertebral disc that leads to the nucleus pulposus, preferably, an opening in the surface of the intervertebral disc that is used for applying the composition to the nucleus pulposus site, namely, an inlet for filling in the composition. Solidification of at least a part of the surface of the composition by gelation can effectively prevent leakage of the composition from the intervertebral disc. A composition-filling inlet on the surface of the intervertebral disc is, for example, preferably an opening formed in the surface of the intervertebral disc with a needle of a syringe or a scalpel for filling in the composition, or an opening in the surface of the intervertebral disc formed with a scalpel or the like upon resection of the herniated disc. In this aspect, an intervertebral disc preferably refers to an annulus fibrosus.

Preferably, the composition of the present invention does not contain a crosslinking agent in an amount that results curing of the composition before application to a nucleus pulposus site of a subject. Therefore, the composition of the present invention may contain a crosslinking agent in an amount that does not result curing of the composition even after a certain period of time. Herein, a certain period of time refers to, but not particularly limited to, preferably about 30 minutes to 12 hours. The phrase “does not contain a crosslinking agent in an amount that results curing of the composition” may be represented, for example, by the composition being injectable with a syringe with a 21 G needle after standing at 20° C. for an hour. The composition of the present invention in some aspects does not contain a crosslinking agent.

There are no particular limitations on the crosslinking agent provided it is able to solidify a surface of a solution of a monovalent metal salt of alginic acid by crosslinking that solution. Examples of the crosslinking agent include divalent or higher valent metal ion compounds such as Ca′, Mg′, Ba 2+, and Sr′, and crosslinking reagents having 2 to 4 amino groups in a molecule thereof. Specific examples of divalent or higher valent metal ion compounds include CaCl₂, MgCl₂, CaSO₄, BaCl₂, and the like, while specific examples of crosslinking reagents having 2 to 4 amino groups in a molecule thereof include diaminoalkanes optionally having a lysyl group (—COCH(NH₂)—(CH₂)₄—NH₂) on a nitrogen atom, namely derivatives which form lysylamino groups as a result of a diaminoalkane and amino group thereof being substituted with a lysyl group. Although specific examples thereof include diaminoethane, diaminopropane and N-(lysyl)-diaminoethane, CaCl₂ solution is particularly preferable for reasons such as ease of acquisition and gel strength.

In one of some aspects of the present invention, the timing of bringing the crosslinking agent into contact with the surface of the composition of the present invention is preferably after the application of the composition of the present invention to the nucleus pulposus site. A method for bringing a crosslinking agent (for example, a divalent or higher valent metal ion) into contact with a part of the composition of the present invention is not particularly limited and may be, for example, a method in which a solution of the divalent or higher valent metal ion is applied to the surface of the composition with a syringe, a spray or the like. For example, a crosslinking agent may continuously and slowly be applied onto the composition-filling inlet formed in the intervertebral disc by spending several seconds to more than 10 seconds. Thereafter, if necessary, a treatment for removing the crosslinking agent remaining in the vicinity of the filling inlet may be added. The crosslinking agent may be removed, for example, by washing the applied part with a physiological saline or the like.

Preferably, the amount of the crosslinking agent used is appropriately adjusted considering the amount of the composition of the present invention applied, the size of the inlet in the surface of the intervertebral disc for filling the composition, the size of the site of the nucleus pulposus of the intervertebral disc to be applied, and the like. In order not to strongly affect the tissue surrounding the composition-filling inlet with the crosslinking agent, the amount of the crosslinking agent used is controlled not to be too much. The amount of the divalent or higher valent metal ion used is not particularly limited as long as the surface of the composition containing the monovalent metal salt of alginic acid can be solidified. When, for example, a 100 mM CaCl₂ solution is used, the amount of the CaCl₂ solution used is preferably about 0.3 ml to 5.0 ml, and more preferably about 0.5 ml to 3.0 ml if the diameter of the filling inlet in the surface of the intervertebral disc is about 1 mm. When the filling inlet in the surface of the intervertebral disc is formed with a scalpel or the like upon resection of the herniated disc with the edges of about 5 mm×10 mm, the amount of the 100 mM CaCl₂ solution used is preferably about 0.3 ml to 10 ml and more preferably about 0.5 ml to 6.0 ml. The amount can suitably be increased or decreased while observing the state of the composition of the present invention at the applied site.

In the case where calcium is contained in the crosslinking agent, a higher calcium concentration is known to result in rapid gelation and the formation of a harder gel. However, since calcium has cytotoxicity, if the concentration is too high, it may have a risk of adversely affecting the nucleus pulposus of an intervertebral disc. Therefore, in the case of using a CaCl₂ solution to solidify the surface of a composition containing a monovalent metal salt of alginic acid, for example, the calcium concentration is preferably set to 25 mM to 200 mM and more preferably 50 mM to 150 mM.

According to the present invention, preferably, the crosslinking agent remaining at the added site after adding the crosslinking agent to the composition and leaving the resultant to stand for a certain period of time, is preferably removed by washing or the like. While the certain period of time for leaving the composition to stand is not particularly limited, it is preferably left to stand for about a minute of longer and more preferably about 4 minutes or longer so as to gel the surface of the composition. Alternatively, it is preferably left to stand for about 1 minute to 10 minutes, more preferably about 4 minutes to 10 minutes, about 4 minutes to 7 minutes, and still more preferably about 5 minutes. The composition and the crosslinking agent are preferably in contact during this certain period of time, and a crosslinking agent may appropriately be added so that the liquid surface of the composition does not dry.

For example, alginate beads can be obtained by dropping a sodium alginate solution into a CaCl₂ solution to form gel. The alginate beads, however, need to be applied by being pressed to the site to be applied and those having a size appropriate for the applied site are required, which is technically difficult in an actual clinical practice. Moreover, when a CaCl₂ solution is used as a crosslinking agent, the Ca ion on the bead surface makes contact with the surrounding tissue, causing a problem of calcium cytotoxicity. On the other hand, the composition of the present invention in a solution state can easily be applied to sites having any kind of shape and can cover the whole area of the site to be applied with good adhesion to the surrounding tissue. The calcium concentration of the part of the composition of the present invention making contact with the surrounding tissue can be kept low and thus the problem of calcium cytotoxicity is little. Since the part of the composition of the present invention making contact with the surrounding tissue is less affected by the crosslinking agent, the composition of the present invention can easily make contact with the cells and the tissue of the site to be applied. Preferably, the composition of the present invention fuses with the tissue of a biological body at the applied site to an unnoticeable level in about 4 weeks after the application to the nucleus pulposus site, with high affinity to a biological body.

When a part of the composition of the present invention is gelled with the crosslinking agent upon applying the composition of the present invention to the nucleus pulposus site, the composition of the present invention is cured at a part of the affected site and localized thereat in the state of being adhered to the surrounding tissue, thereby preventing leakage from the nucleus pulposus site. In addition, as a result of adhering the composition of the present invention to the surrounding tissue, the effects of the composition of the present invention to suppress pain and/or inflammation at a surgical site and/or a surrounding site thereof can be demonstrated more potently.

When the filling material replenishing the nucleus pulposus site was entirely gelled and cured, a phenomenon where the cured gel deviated from the composition filling inlet on the surface of the intervertebral disc was observed when compression force was placed on the intervertebral disc from the head and tail sides. On the other hand, when the composition in a solution state was used to fill the nucleus pulposus site, there was no deviation from the filling inlet in the surface of the intervertebral disc even when compression force was placed from the head and tail sides. That is, it can be said that the risk of the replenished composition to leak out is little even against compression to the intervertebral disc from the vertical direction when the composition of the present invention is actually used for filling the nucleus pulposus.

Furthermore, when a cured gel fills the nucleus pulposus site, the cured gel may have a risk of protruding into the spinal canal, which may cause serious neuropathy. On the other hand, the composition of the present invention in a solution state is hardly associated with such a risk with little risk of onset of complications.

In one aspect of the present invention, a carrier for embedding cells may be used without curing. It may be applied without using a crosslinking agent, depending on clinical symptoms and the size and shape of the injured area.

### 11. Application of Composition of the Present Invention

The composition of the invention is applied to the nucleus pulposus site of the intervertebral disc in humans or non-human organisms, for example, in birds and non-human mammals (for example, bovines, monkeys, cats, mice, rats, guinea pigs, hamsters, swines, dogs, rabbits, sheep, and horses), and is used to promote regeneration of the nucleus pulposus.

The composition of the present invention is preferably in a liquid state having fluidity, namely, in a solution state. In the present invention, the phrase “having fluidity” refers to having of a property that causes the form thereof to change to an amorphous form, and does not require that the form constantly have the property of flowing in the manner of a liquid, for example. Preferably, it has fluidity that allows the composition to be sealed in a syringe and injected into a nucleus pulposus site of an intervertebral disc. Furthermore, in one of some aspects of the present invention, the composition preferably has fluidity to be injected into a nucleus pulposus site of an intervertebral disc with a syringe with a 14 G to 26 G needle, more preferably a 21 G needle, after being left to stand at 20° C. for an hour. When the composition of the present invention is provided in a dry state as a lyophilizate or the like, it can be made into a composition to have the above-described fluidity with a solvent or the like upon application. The composition of the present invention in a solution state can easily be applied to a nucleus pulposus site of an intervertebral disc with a syringe, a pipette for gel, a specialized syringe, a specialized injector, a filling tool or the like.

Since application with a syringe is difficult when the viscosity of the composition of the present invention is high, a pressurized or electric syringe or the like may be used. Even without a syringe or the like, application to a defective part of the nucleus pulposus site can be carried out, for example, with a spatula, a stick or the like. When a syringe is used for injection, for example, a 14 G to 27 G or 14 G to 26 G needle is preferably used.

While the method for applying the composition of the present invention to the nucleus pulposus site is not particularly limited, the composition of the present invention is preferably applied to the nucleus pulposus site by using a syringe, a filling tool or the like after exposing the affected site by a known surgical process under direct vision, or under a microscope or an endoscope. In one preferable aspect, a needle of a filling tool or the like can be inserted from the surface of the annulus fibrosus toward the nucleus pulposus site to apply the composition of the present invention.

Since the composition of the present invention is in the form of a solution, it can suit a nucleus pulposus site with any shape including shrinkage of the nucleus pulposus and a cavity or a defective part of the nucleus pulposus site such that it can fill the entire shrinkage, cavity, or defective part of the nucleus pulposus. The shrinkage of the nucleus pulposus and the cavity and the defective part of the nucleus pulposus site may result from degeneration or injury of the intervertebral disc or upon removal or suction of at least a part of the nucleus pulposus by a surgical operation. Preferably, the composition of the present invention is applied to a nucleus pulposus defective part that is formed by removing at least a part of the nucleus pulposus.

While the removal of at least a part of the nucleus pulposus is not particularly limited, it may, for example, be an intervertebral discectomy or the like performed under direct vision, transdermally, under microscopic vision or endoscopically. Alternatively, it may be, for example, a method in which an incision of 2 cm to 10 cm is made in the back to remove the muscle from the rear surface of the posterior element of the vertebral column called a vertebral arch to resect the ligament between the vertebral arches, confirm the nerve and disc herniation, and excise the hernia pressurizing the nerve (Love's method). Alternatively, the method may be one in which the nucleus pulposus is irradiated with laser to reduce the volume of the nucleus pulposus.

After the application of the composition of the present invention to the nucleus pulposus site, the composition can partially be cured with a crosslinking agent as described above.

While the amount of the composition of the present invention applied is not particularly limited and can be determined according to the volume of the applied site of the nucleus pulposus of the subject to be applied, it may, for example, be 0.01 ml to 10 ml, more preferably, 0.1 ml to 5 ml, and still more preferably 0.2 ml to 3 ml. When the composition of the present invention is applied to the nucleus pulposus defective part, it is preferably injected so as to sufficiently fill the volume of the defective part of the nucleus pulposus site.

The number of times and the frequency of the application of the composition of the present invention can be increased or decreased according to the symptoms and the effect. For example, it may be a single application, or regular application once in a month to a year.

Since an alginic acid does not naturally exist in the bodies of animals, animals do not possess an enzyme to specifically degrade the alginic acid. While an alginic acid can be gradually degraded in an animal body due to general hydrolysis, its degradation in the body is milder as compared to a polymer such as hyaluronic acid. In addition, since no blood vessel exists in the nucleus pulposus, the effect of the alginic acid is expected to last long when filled inside the nucleus pulposus.

Even in a case where the composition of the present invention is not provided together with the aforementioned cells and growth factors, when the composition of the present invention is applied to the nucleus pulposus site, the aforementioned cells, growth factors, cell death-suppressing factors, the after-mentioned other agents, and the like may be used in combination with the present composition.

By applying the composition of the present invention to the nucleus pulposus site, the present composition exhibits the effect of suppressing degenerative changes of the entire intervertebral disc tissues and the nucleus pulposus and promoting regeneration. Thus, the composition of the present invention is preferably used as a composition for filling of the nucleus pulposus of an intervertebral disc.

In a preferred aspect, the composition of the present invention is a composition for suppression of degeneration of an intervertebral disc, and is more preferably a composition for suppression of degeneration of the nucleus pulposus of an intervertebral disc. The “degeneration of an intervertebral disc or nucleus pulposus” means a condition in which the number of cells in the intervertebral disc, water contents, extracellular matrixes (type II collagen, aggrecan, etc.) and the like decrease due to aging, etc., resulting in morphological changes and functional decline. The progression of the degeneration results in the inability of the intervertebral disc to function as a shock absorber. In the present description, “suppression of degeneration” may be defined to be the suppression of degenerative changes compared with an untreated condition, and does not necessarily mean the absence of degeneration.

In one aspect, the composition of the present invention is a composition for regeneration of the nucleus pulposus. Regeneration of the nucleus pulposus is directed towards preventing accumulation of fibroblast-like cells and regenerating a nucleus pulposus with a high percentage of nucleus pulposus cells, and thus, regeneration of the nucleus pulposus is intended to regenerate nucleus pulposus tissues that are rich in type II collagen and proteogly can. The term “regeneration of the nucleus pulposus” also includes suppression of degeneration of the nucleus pulposus. In one preferred aspect of the present invention, the composition of a nucleus pulposus that is regenerated by application of the composition of the present invention is desirably close to the composition of a natural normal nucleus pulposus.

In addition, in a preferred aspect, the composition of the present invention is used for the treatment, prevention, or recurrence suppression of intervertebral disc degeneration and/or intervertebral disc damage. In the present description, the “treatment, prevention, or recurrence suppression” includes treatment, prevention, suppression of recurrence, reduction, suppression, improvement, removal, reduction in incidence, delay of onset time, suppression of progression, reduction in severity, reduction in recurrence rate, delay of recurrence time, alleviation of clinical symptoms, etc. In the present description, the “treatment, prevention, or recurrence suppression” includes alleviation of (chronic) pain. In a preferred aspect, in particular, in the case of using a carrier for embedding cells without curing it, the composition of the present invention is used to suppress (chronic) pain (in particular, low back pain) associated with intervertebral disc degeneration and/or intervertebral disc damage.

These preferred aspects of the composition of the present invention, the methods of using the present composition, and the like are as those described above.

The intervertebral disc degeneration and/or the intervertebral disc are at least one condition or disease selected from the group consisting of intervertebral disc herniation, discopathy, degenerative spondylolisthesis, pyogenic discitis, spondylosis deformans, spinal canal stenosis, lumbar spinal stenosis, intervertebral disc damage, and intervertebral disc herniation associated with lumbar spinal stenosis (also referred to as “combined lumbar spinal stenosis”). The intervertebral disc degeneration and/or the intervertebral disc damage may also be attended with low back pain.

The composition of the present invention may also be used without curing a carrier for embedding cells, depending on clinical symptoms and the size and/or shape of the damaged area. In such an aspect, the composition of the present invention is used to suppress pain associated with the intervertebral disc degeneration and/or the intervertebral disc damage, in particular, chronic low back pain.

### 12. Therapeutic Method

The present invention provides a method for the treatment, prevention, or recurrence suppression of intervertebral disc degeneration and/or intervertebral disc damage, in which the above-described composition of the present invention is used. Preferably, the therapeutic method of the present invention is a method for the treatment, prevention, or recurrence suppression of intervertebral disc degeneration and/or intervertebral disc damage, wherein said method comprises applying a composition comprising a low endotoxin monovalent metal salt of alginic acid and having fluidity to the nucleus pulposus site of the intervertebral disc of a subject that is in need of the treatment, prevention, or recurrence suppression.

The therapeutic method of the present invention may comprise a step of removing at least a part of the nucleus pulposus, before application of the composition of the present invention to the nucleus pulposus site.

The intervertebral disc degeneration and/or the intervertebral disc damage are, for example, at least one condition or disease selected from the group consisting of disc herniation, discopathy, degenerative spondylolisthesis, pyogenic discitis, spondylosis deformans, spinal canal stenosis, and intervertebral disc injuries. According to the therapeutic method of the present invention in some aspects, the intervertebral disc degeneration and/or the intervertebral disc injury is disc herniation, and particularly lumbar disc herniation. In several aspects of the present invention, in the present therapeutic method, the intervertebral disc degeneration and/or the intervertebral disc damage are intervertebral disc herniation associated with lumbar spinal stenosis (also referred as “combined lumbar spinal stenosis”). The intervertebral disc degeneration and/or the intervertebral disc damage may be chronic low back pain. The intervertebral disc degeneration and/or the intervertebral disc damage may also be a combination of these conditions or diseases.

Moreover, in one of several aspects, the present invention provides a method for suppressing degenerative changes of an intervertebral disc, in which the above-described composition of the present invention is used. Furthermore, in one of preferred aspects, the present invention provides a method for regenerating the nucleus pulposus of an intervertebral disc, in which the above-described composition of the present invention is used.

These methods comprise applying a composition comprising mesenchymal stem cells and a low endotoxin monovalent metal salt of alginic acid and having fluidity to the nucleus pulposus site of the intervertebral disc of a subject that is in need of suppression of degeneration of the intervertebral disc or regeneration of the nucleus pulposus, and then curing a part of the applied composition. The above-described methods may comprise a step of removing at least a part of the nucleus pulposus, before application of the composition of the present invention to the nucleus pulposus site.

Preferred aspects of the composition of the present invention, specific methods of applying the present composition to the nucleus pulposus site of the intervertebral disc, the methods of curing the present composition, the meanings of the terms, etc. are as those described above. The therapeutic method of the present invention may also be carried out by being appropriately combined with other therapeutic methods and therapeutic agents for intervertebral discs.

Furthermore, a co-administered drug, for example, an antibiotic such as streptomycin, penicillin, tobramycin, amikacin, gentamycin, neomycin or amphotericin B, an anti-inflammatory agent such as aspirin, a non-steroidal anti-inflammatory drug (NSAID) or acetaminophen, a proteinase, a corticosteroid drug or a HMG-CoA reductase inhibitor such as simvastatin or lovastatin may be filled before, simultaneous to or after application of the composition of the present invention to the nucleus pulposus site. These drugs may also be used in a mixing into the composition of the present invention. Alternatively, they may be administered orally or parenterally for co-administration. In addition, if necessary, a muscle relaxant, an opioid analgesic, a neurogenic pain alleviating drug or the like may be administered orally or parenterally for co-administration.

The present invention also relates to use of a low endotoxin monovalent metal salt of alginic acid for production of the composition of the present invention.

The use of the present invention is use of a low endotoxin monovalent metal salt of alginic acid for production of a composition for use in the treatment, prevention, or recurrence suppression of intervertebral disc degeneration and/or intervertebral disc damage, wherein said composition is used, such that the composition is applied to the nucleus pulposus site of a subject, and a part of the composition is cured after the application, and said composition has fluidity upon the application thereof to the nucleus pulposus site.

The present invention further provides a low endotoxin monovalent metal salt of alginic acid that is used in the treatment, prevention, or recurrence suppression of intervertebral disc degeneration and/or intervertebral disc damage, in which a composition comprising mesenchymal stem cells and a low endotoxin monovalent metal salt of alginic acid and having fluidity is applied to the nucleus pulposus site of the intervertebral disc of a subject that is in need of the treatment, prevention, or recurrence suppression of intervertebral disc degeneration and/or intervertebral disc damage, and a part of the applied composition is then cured.

### 13. Evaluation of Composition of the Present Invention

The composition of the present invention can be evaluated using a severe intervertebral disc degeneration sheep model that has been newly established by the present inventors. Such a severe intervertebral disc degeneration model can be produced by (a) removing nucleus pulposus tissues in an amount corresponding to 0.00004% to 0.00005% of the sheep body weight from a sheep intervertebral disc in a first surgery, to produce a degenerated intervertebral disc, and (b) by further removing nucleus pulposus tissues in an amount corresponding to 0.00014% to 0.000175% of the sheep body weight from the degenerated intervertebral disc produced in the above (a) 4 weeks after the first surgery, so as to produce a severe intervertebral disc degeneration sheep model.

In a preferred aspect, such a severe intervertebral disc degeneration sheep model can be produced by (a) removing 20 mg of nucleus pulposus tissues from the intervertebral disc of a sheep having a body weight of 40 to 50 kg in a first surgery, to produce a degenerated intervertebral disc, and (b) by further removing 70 mg of nucleus pulposus tissue from the degenerated intervertebral disc produced in the above (a), 4 weeks after the first surgery, so as to produce a severe intervertebral disc degeneration sheep model.

The composition of the present invention can be evaluated according to the following procedures (a) to (d). (a) In a first surgery, 20 mg of nucleus pulposus tissues are removed from a sheep intervertebral disc to produce a degenerated intervertebral disc; (b) in a second surgery, 70 mg of nucleus pulposus tissues are further removed from the degenerated intervertebral disc produced in the above (a) to produce a severe intervertebral disc degeneration sheep model 4 weeks after the first surgery; (c) after the second surgery, a subject composition is administered to a generated void; and (d) after administration of the composition, a vertebral body and an intervertebral disc that are collected from the degeneration model are evaluated according to at least one evaluation method selected from the group consisting of MRI, histological staining, and immunohistochemical staining (IHC), in terms of regeneration of the intervertebral disc.

In the case of previous large animal models, intervertebral disc degeneration models were produced by partially excising a normal intervertebral disc from the large animal models. Accordingly, it was likely that the intervertebral disc would be naturally healed. However, by using the present animal model, from which an already degenerated intervertebral disc is partially excised, the effects of the present examples against the degenerated intervertebral disc of a human can be more precisely evaluated.

### EXAMPLES

Hereinafter, the present invention will be more specifically described in the following examples. However, these examples are not intended to limit the scope of the present invention.

### Example 1

### Intervertebral Disc Damage Treatment Test Using Intervertebral Disc Damage Rabbit Models

In the present example, using a composition comprising bone marrow-derived mesenchymal stem cells (BMSCs) and bioabsorbable ultra-purified alginate (also referred to as “low endotoxin high purity alginate” or “UPAL”) gel, the regenerative effects of BMSC transplantation against a degenerated IVD after excision of an intervertebral disc (IVD) were examined.

### 1. Materials and Methods

### 1.1. Animal Experiments

All procedures to animals were approved by the Animal Care and Use Committee of Hokkaido University (Approval No.: 13-0051), and were performed according to the approved guidelines. Male Japanese white rabbits (20 weeks old, 3.2 to 3.5 kg) were obtained from Sankyo Labo Service Corporation, Inc. (Tokyo, Japan).

### 1.2. Preparation of Rabbit Nucleus Pulposus Cells (NPCs)

Nucleus pulposus (NP) samples were obtained by subjecting 4 rabbits to euthanasia via administration with an excessive amount of intravenous pentobarbital, and then collecting the samples from their lumbar IVDs (L1/2 to L5/6; 20 IVDs in total). NPCs were isolated from the nucleus pulposus (NP) tissues and were then cultured according to the methods of previous reports [2, 5, 7, and 8]. Specifically, gel-like NP tissues were separated from annulus fibrosis (AF) using micro forceps under sterile conditions. Tissue specimens were placed in a culture medium containing Dulbecco's Modified Eagle Medium (Sigma-Aldrich, St. Louis, MO, USA) supplemented with 10% fetal bovine serum (Nichirei Bioscience, Tokyo, Japan), 1% penicillin/streptomycin, and 1.25 mg/ml fungizone (Life Technologies, Waltham, MA, USA). No exogenous growth factors were used. The samples were resuspended in a medium supplemented with 0.25% collagenase (Wako Pure Chemical Industries, Ltd., Osaka, Japan), and were then incubated at 37° C., in 20% 02 and 5% CO2, for 4 hours in a shaking incubator. Thereafter, the resulting cells were isolated by enzymatic digestion. The cells isolated from the NP tissues were allowed to proliferate in a culture dish and were then cultured in the above-described medium at 37° C. containing 20% 02 and 5% CO2 in humidified air. The medium was exchanged twice a week and the NPCs were used at passage 2.

### 1.3. Preparation of Rabbit Allogeneic BMSCs

OriCell™ rabbit mesenchymal stem cells were purchased from Cyagen (Santa Clara, CA, USA; catalogue number: RBXMX-01,001, Lot No.: 151114131) and were used as rabbit allogeneic BMSCs. These cells had been tested for characteristics, viability after thawing, cell cycle, verification of an undifferentiated state, and pluripotent differentiation ability along osteogenic, chondrogenic, and adipogenic lines. The BMSCs were cultured according to the manufacturer's instructions, the medium was exchanged twice a week, and the BMSCs were used at passage 2.

### 1.4. Preparation of UPAL Gel and Three-Dimensional (3D) Culture

In the present example, UPAL gel (Mochida Pharmaceutical Co. Ltd., Tokyo, Japan) was used as an alginate scaffold for 3D culture [2]. The purification process of UPAL gel is as previously reported [2]. Specifically, the alginate in seaweeds was extracted by converting it to water-soluble sodium alginate according to a clarification procedure [2]. Since this alginate solution had high viscosity, it was diluted with a large amount of water [2]. Subsequently, the extract was filtrated to separate a sodium alginate solution from a fibrous residue [2]. In order to isolate high quality alginic acid, an acid was added to this solution [2].

A 2% (w/v) UPAL solution dissolved in a phosphate buffered saline (Wako Pure Chemical Industries, Ltd.) and 102 mM CaCl₂ was prepared for gelling. Before a 3D culture, BMSCs were fluorescently labelled with 5,6-carboxyfluorescein diacetate succinimidyl ester (CFDA-SE; CFDA-SE Cell Proliferation Assay Kit; BIO RAD, Hercules, CA, USA) having a final concentration of 20 mM, according to the manufacturer's instructions [9]. Subsequently, the labelled BMSCs and non-labelled NPCs were embedded in the UPAL solution at a ratio of 1:1 (1×10⁶ cells/ml each) [1 and 10], to obtain a final cell concentration of 2×10⁶ cells/ml [9 and 10]. The UPAL/cell mixture was placed through a 22-gauge needle into 102 mM CaCl₂ for gelling. Gel beads were cultured in the above-described medium under hypoxic conditions (5% 02 and 5% CO2) for 7 days [10]. Furthermore, NPCs and BMSCs were separately embedded in the UPAL solution at a cell concentration of 1×10⁶ cells/ml. The cell concentration was set based on a previously reported example [11], in which effects were compared using total cell numbers that were different in individual groups. After gelling, the gel beads were cultured under hypoxic conditions as described above. The experimental groups were as follows: (a) NPC monoculture; (b) BMSC monoculture; and (c) NPC+BMSC co-culture.

At 0 and 7 days after the culture, all gel beads were dissolved in 55 mM sodium citrate until the gel and the cells were separated according to a previously reported method [8]. In the NPC+BMSC co-culture group, the recovered cells were sorted using a BD FACS Aria III High speed cell sorter with Diva software version 7.0 (BD Biosciences, San Jose, CA, USA) [1 and 12]. Dead cells and residues were removed, and cells emitting fluoresce at 530 nm were identified as BMSCs, whereas non-fluorescent cells were identified as NPCs.

1.5. RNA Extraction and Real-Time Quantitative Reverse Transcription Polymerase Chain Reaction (qRT-PCR)

The recovered NPCs and BMSCs were dissolved in 1 ml of TRIzol™ (Invitrogen, Carlsbad, CA, USA), and total RNA was then extracted from the samples using the RNeasy Mini kit (Qiagen, Valencia, CA, USA). Real-time qRT-PCR analysis was performed using a TaqMan™ gene expression assay and a custom TaqMan™ gene expression assay (Table 1) (Applied Biosystems, Waltham, MA, USA). A cycle threshold (Ct) was obtained for each sample, and the relative mRNA expression of each target gene was then calculated with reference to the Ct value of the housekeeping gene GAPDH, using a 2^{-ΔCt} method [1].

TABLE 1
Predesigned primer and probe mixes
by Applied Biosystems for qRT-PCR

| Gene | Gene symbol | Assay ID |
|---|---|---|
| HIF-1a | HIF1A | Oc03398626_m1 |
| GLUT-1 | SLC2A1 | Oc03399482_m1 |
| Brachyury | T | Oe03395780_m1 |
| CDMP-1 | GDFS | Oc00433564_m1 |
| TGF-β | TGFB1 | Oc04176122_u1 |
| IGF-1 | IGF1 | Oc04096599_m1 |
| Type II collagen | COL2Al | Oc03396134_m1 |
| Aggrecan * | ACAN | — |

* Custom TaqMan ® Gene Expression Assay

### 1.6. In Vivo Test Using Rabbit Degenerated IVD Models

A total of 48 rabbits were used in an in vivo test. The sample size was determined on the basis of previous reports [2, 7, and 8] for each of the two time points employed. Thirty-two out of the 48 rabbits were randomly selected, and qualitative analyses of IVD degeneration (magnetic resonance imaging (MRI), histology, and immunohistochemistry (IHC)) were carried out. A total of 80 IVDs were randomly assigned to an intact control group, a puncture group (puncture performed only to create degeneration), a discectomy group (partial discectomy to create cavities on degenerated IVDs), a gel group (partial discectomy and UPAL gel embedding to degenerated IVDs), and a BMSCs+gel group (partial discectomy and the embedding of BMSCs and UPAL gel to degenerated IVDs) (8 IVDs per group).

Using frozen sections of 4 rabbit NP tissues, the survival of the transplanted BMSCs was evaluated, and a total of 8 IVDs were randomly assigned to the intact control group and the BMSCs+gel group (4 IVDs per group). The remaining 12 rabbits were used to evaluate the expression of HIF-1α, GLUT-1 and Brachyury. These markers have been proposed as major markers that are expressed in healthy human NPCs (in which IHC of paraffin sections of rabbit NP tissues was used). A total of 36 IVDs were randomly assigned to the intact control group, the discectomy group, and the BMSCs+gel group (4 IVDs per group).

### 1.7. BMSCs Labeling and Embedding in UPAL Solution

In an in vivo experiment, the same OriCell′ rabbit mesenchymal stem cells as those used in the in vitro experiment were used at passage 2 as cells to be transplanted. BMSCs were labelled with CFDA-SE before transplantation, and were then embedded in a 2% UPAL solution, so as to adjust to a final cell concentration of 1×10⁶ cells/ml [14].

### 1.8. Production of Degenerated IVDs and Cell Transplantation

Twenty-week-old rabbits are not sufficiently aged to mimic the condition of an aged human population, and, it is difficult to obtain aged rabbits with uniform ageing. Hence, in the present example, in order to obtain degenerated IVDs, annulus fibrosis (AF) puncture models of rabbit intervertebral discs were used [8, 15, and 16]. Rabbits were subjected to general anesthesia by intravenous injection of ketamine (10 mg/kg) and xylazine (3 mg/kg), and then, the anesthesia was maintained with 02 and air (3.0 l/min) mixed with sevoflurane (2% to 3%) under spontaneous ventilation. Degenerated IVDs were created from L2/3 and L4/5 IVDs by AF puncture using an 18-gauge needle. L3/4 IVD was untreated and was used as a control.

At 4 weeks after the IVD puncture, a UPAL solution containing BMSCs was transplanted into the sites of the L2/3 and L4/5 degenerated IVD [8]. Under general anesthesia, the spine was exposed by an anterolateral retroperitoneal approach. In the discectomy group, the gel group, and the BMSCs+gel group, after puncturing with an 18-gauge needle to create a puncture entrance, degenerated NP tissues were aspirated using a 10 ml syringe, and the remaining NP tissues were removed using micro forceps (Nagashima Medical Instruments Co., Ltd., Tokyo, Japan) with L2/3 and L4/5 IVDs (about 10 to 12 mg (wet weight) per IVD), so as to create an IVD cavity. L3/4 IVD was untreated and was used as a control.

In the gel group, using a microsyringe (Hamilton Medical, Bonaduz, Switzerland) fitted with a 27-gauge needle, the IVD defective site was filled with 20 μl of a 2% UPAL solution, whereas in the BMSCs+gel group, the IVD defective site was filled with a UPAL solution containing BMSCs. In particular, since a 26-gauge needle had been demonstrated to have no effects on cell viability or tissue degeneration, a 27-gauge needle was used herein [2, 7, 8, and 17]. Next, 1 mL of 102 mM CaCl₂ was sprayed onto the UPAL solution to induce gelling. Five minutes later, a surgical wound was washed with a normal saline and was closed. In the puncture group, a sham surgery was performed. At 4 weeks and 12 weeks after the surgery for analysis of IVD degeneration, or 1, 7 and 28 days after the surgery for evaluation of the expression of NPC markers, rabbits were sacrificed by intravenous administration with an excessive amount of pentobarbital. In order to evaluate the effects of human BMSCs on rabbit IVDs, bone marrow-derived human mesenchymal stem cells (hMSC-BM; PromoCell, Heidelberg, Germany; catalogue No. C-12974, Lot No.: 412Z022.4) were used to perform the same embedding experiment as described above.

### 1.9. Evaluation of Survival of Transplanted BMSCs

At 4 weeks and 12 weeks after the embedding, the survival of the embedded BMSCs was confirmed based on CFDA-SE fluorescence labelling [18 and 19]. IVDs (an intact control and a BMSCs+gel group) were cut horizontally in half, were then frozen in liquid nitrogen, and were then sectioned into 5 mm slices. Thereafter, as contrast staining, the sliced IVDs were stained with 4′,6-diamidino-2-phenylindole (DAPI; Invitrogen; P36935). In particular, in the case of DAPI-positive cells, NPCs and BMSCs that survived or died could not be specified. Accordingly, the viability of the transplanted BMSCs could not be quantified.

### 1.10. MRI Analysis

At 4 weeks and 12 weeks after the surgery, using a 7.0-T MR scanner (Varian Unity Inova; Varian Medical Systems, Palo Alto, CA, USA), T2-weighted mid-sagittal section images of the IVDs were obtained [2, 8, and 16]. Using Pfirrmann classification (in which grade 5 was classified as severe degeneration), IVD

degeneration was graded [20]. In order to calculate an MRI index, quantitative analysis was also performed using analysis software version 12.0 (AnalyzeDirect, Overland Park, KS, USA). The MRI index (a product of an NP area and a mean signal intensity) was applied to quantification of NP degeneration, and the quantitative data were expressed as a percentage of the obtained value to the MRI index obtained in an untreated control IVD (relative MRI index) [2, 8, and 16].

### 1.11. Histological Analysis

After the MRI analysis, each IVD was treated for histological staining. In order to evaluate proteoglycan expression, mid-sagittal sections (5 mm thick) were stained with hematoxylin and eosin (H & E) and also with safranin 0-fast green [8]. Semi-quantitative analysis was performed on the IVDs, and the IVDs were graded from 0 (normal) to 5 (highly degenerated) [21 and 22]. Specifically, this histological scale focuses on morphological changes in AF structures.

### 1.12. IHC Analysis

At 4 weeks and 12 weeks after the surgery, in order to detect type I and type II collagens, immunohistochemical (IHC) staining was performed [8], and at 1, 7 and 28 days after the surgery, HIF-1α, GLUT-1 and Brachyury were detected. For the staining of type I and type II collagens, mouse monoclonal antibodies were applied against type I collagen (Sigma-Aldrich; C2456, RRID: AB_476836) and type II collagen (Kyowa Pharma Chemical, Toyama, Japan; F-57). The staining was performed using 3,3′-diaminobenzidine hydrochloride (Dako) and Mayer's hematoxylin (Merck, Darmstadt, Germany) as contrast staining. With regard to HIF-1α, GLUT-1 and Brachyury staining, a DyLight 550-conjugated rabbit polyclonal antibody (Novus Biologicals, Centennial, CO, USA; NB100-479R, RRID: AB_1642267) reacting against HIF-1α, a PE-conjugated rabbit polyclonal antibody (LS Bio, Seattle, WA, USA; LS-A109342-100) reacting against GLUT-1, and a non-conjugated rabbit polyclonal antibody (LS Bio; LS-C31179-100, RRID: AB_911118) reacting against Brachyury were applied. Furthermore, an Alexa Fluor 594-conjugated goat anti-rabbit polyclonal antibody (Invitrogen; A32740) was used as secondary antibody for Brachyury. The staining was developed using DAPI as contrast staining.

Cells that were positive to type I and type II collagens, HIF-1α, GLUT-1 and Brachyury were measured, separately, in five independent, randomly selected fields of view [2 and 8]. The fields of view extend to the width of the NP including both deep and surface regions. The numerical value is indicated with the percentage of the number of the positive cells to the total number of cells in all of evaluation times, and is also indicated with the percentage to CFDA-SE positive cells for evaluation of the NPC marker. All experiments were performed on 8 IVDs for type I and type II collagen evaluation, and on 4 IVDs for NPC marker evaluation from each treatment group, at each time point.

### 1.13. Statistical Analysis

All data are shown in the form of a mean value±standard error (SE). One-way analysis of variance (ANOVA) and Tukey-Kramer post-test were performed for multi-group comparisons. For two-group comparisons, Paired t-test was used. All statistical calculations were performed using JMP Pro-version 14.0 statistical software (SAS Institute, Cary, NC, USA) with a significance threshold of p<0.05.

### 2. Results

2.1. Promotion of Differentiation of BMSCs into NPCs and Generation of Growth Factors and ECMs by Co-Culture of NPCs and BMSCs

In order to examine the effects of the co-culture of NPCs and BMSCs on each cell type, non-labelled NPCs and CFDA-SE-labelled BMSCs were embedded in UPAL gel for 3D culture. The present inventor collected both cell types in the co-culture group, using a cell sorter. Phosphate-buffered saline/cell suspension analysis was performed using forward scattering and side scattering. P1 gates were plotted by 2D dot plotting (FIG. 1a), and dead cells and residues were excluded. Non-labelled NPCs and CFDA-SE-labelled BMSCs were sorted using different gates in a fluorescence versus side scatter dot plot (FIG. 1b). Accordingly, P2 gates were set on non-labelled cells, and P3 gates were set on CFDA-SE-labelled cells, and in order to avoid cross contamination, a gap was established between the two types of gates [1]. Following gel lysis and cell sorting, 6 types of cells were obtained, namely, (a) NPC control (day 0), (b) NPC monoculture, (c) NPC co-culture, (d) BMSC control (day 0), (e) BMSC monoculture, and (0 BMSC co-culture. For the analysis of BMSC differentiation, the gene expression of HIF-1α, GLUT-1 and Brachyury used as NPC markers, the gene expression of CDMP-1, TGF-β and IGF-1 used as growth factors, and the gene expression of type II collagen and aggrecan used as extracellular matrixes (ECMs) were evaluated in the 6 types of cells according to qRT-PCR.

The gene expression of HIF-1α in the NPC co-culture was significantly increased, compared with the NPC control (p=0.0218, Tukey-Kramer test), and in the BMSC co-culture, the gene expression of HIF-1α was significantly increased, compared with the BMSC control (p=0.0041, Tukey-Kramer test) and with the BMSC monoculture (p=0.0041, 0.0116, Tukey-Kramer test) (FIG. 1c).

The expression of GLUT-1 in the NPC co-culture showed a significant increase, compared with the NPC control (p<0.0001, Tukey-Kramer test) and with the NPC monoculture (p<0.0001, Tukey-Kramer test). The expression of GLUT-1 in the NPC monoculture showed a significant increase, compared with the NPC control (p<0.0001, Tukey-Kramer test). The expression of GLUT-1 in the BMSC co-culture showed a significant increase, compared with the BMSC control (p=0.0189, Tukey-Kramer test) (FIG. 1d). In contrast, the gene expression of Brachyury did not show a statistically significant difference among the three types of NPCs. Furthermore, the gene expression of Brachyury was observed only in the BMSC co-culture, and was not observed in either the BMSC control or the BMSC monoculture (FIG. 1e).

The gene expression of CDMP-1, TGF-β and IGF-1 in the NPC co-culture was significantly increased, compared with the NPC control (p<0.0001, p<0.0001, p=0.0002, Tukey-Kramer test) and with the NPC monoculture (p<0.0001, p<0.0001, p=0.0082, Tukey-Kramer test). The gene expression of CDMP-1, TGF-β and IGF-1 in the NPC monoculture was significantly increased, compared with the NPC control (p=0.0179, p<0.0001, p=0.0079, Tukey-Kramer test). The gene expression of CDMP-1, TGF-β and IGF-1 in the BMSC co-culture was significantly increased, compared with the BMSC control (p=0.0011, p<0.0001, p<0.0001, Tukey-Kramer test) and with the BMSC monoculture (p=0.0015, p=0.0386, p<0.0001, Tukey-Kramer test) (Figure if-h).

The expression of type II collagen in the NPC co-culture and mono-culture was significantly increased, compared with the NPC control (p=0.0036, p=0.0035, Tukey-Kramer test). The expression of type II collagen in the BMSC co-culture was significantly increased, compared with the BMSC control (p=0.004, Tukey-Kramer test) and with the BMSC monoculture (p=0.0226, Tukey-Kramer test) (FIG. 1i). Regarding aggrecan, the gene expression thereof in the NPC co-culture was significantly increased, compared with the NPC control (p=0.0047, Tukey-Kramer test) and the NPC monoculture (p=0.0392, Tukey-Kramer test). The expression of aggrecan in the NPC monoculture was significantly increased, compared with the NPC control (p=0.0274, Tukey-Kramer test). In particular, aggrecan was only expressed in the BMSC co-culture, and it was not expressed in the BMSC control or the BMSC monoculture (FIG. 1j).

### 2.2. Survival of Transplanted BMSCs

In an in vivo test, in the BMSCs+gel group, CFDA-SE-labelled BMSCs were observed, but were not observed in the intact control group at 4 weeks and 12 weeks after the surgery (FIG. 2). The human BMSCs group also showed similar findings to the BMSCs+gel group (rabbit BMSCs), and it was confirmed that the transplanted BMSCs survived in IVDs at 12 weeks after the transplantation. When incised, no gel extrusion was observed.

2.3. Combined Use of BMSCs and UPAL Gel that Promotes IVD Regeneration in Degenerated IVDs after Discectomy

Degenerative changes in the treated IVDs were qualitatively analyzed by MRI, and T2-weighted mid-sagittal section images were captured (FIG. 3a). The Pfirrmann grade in the BMSCs+gel group was significantly lower than in the discectomy group at 4 weeks (p=0.003, Tukey-Kramer test), and was significantly lower in the puncture group and the discectomy group at 12 weeks (p=0.0009, p<0.0001, Tukey-Kramer test). Furthermore, the degree of degeneration in the gel group was also significantly lower than in the discectomy group at 12 weeks (p=0.0028, Tukey-Kramer test) (FIG. 3b). No significant difference was observed between the gel group and the BMSCs+gel group. The MRI index of the BMSCs+gel group was significantly higher than the discectomy group at 4 weeks (p=0.0085, Tukey-Kramer test), and was significantly higher than the puncture group, the discectomy group, and the gel group at 12 weeks (p=0.0002, p<0.0001, p=0 to 0248, Tukey-Kramer test). In addition, the index of the gel group was significantly higher than that of the discectomy group at 12 weeks (p=0.0092, Tukey-Kramer test) (FIG. 3c). In particular, no significant difference was observed between the rabbit BMSCs group and the human BMSCs group, in terms of either the Pfirrmann grade or the MRI index.

Prior to histological degeneration grade classification, the overall structure of IVD including NP and AF was evaluated, and gross differences were observed among the groups. As a result of the histological evaluation of the IVD, it was found that the intact control specimens were not accompanied with structural collapse of the internal AF, and that the NP tissues showed a typical oval shape (FIGS. 4, a and b). In the discectomy group, AF collapsed inside, and fibrotic changes in the NP tissues were observed at both 4 weeks and 12 weeks. However, the internal AF in the BMSCs+gel group remained with minimal fibrotic changes in the NP tissues, and appeared to be favorably preserved at both weeks 4 and 12. The internal AF in the gel group also appeared to be relatively favorably preserved (FIGS. 4, a and b). The histological degeneration score of the BMSCs+gel group was significantly lower than that of the puncture group (p=0.0006, p<0.0001, Tukey-Kramer test), the discectomy group (p<0.0001, p<0.0001, Tukey-Kramer test), and the gel group (p=0.00134, p<0.0001, Tukey-Kramer test). At 12 weeks, the score of degeneration in the gel group was significantly lower than that of the discectomy group (p=0.0006, Tukey-Kramer). Moreover, there was no significant difference between the rabbit BMSCs group and the human BMSCs group. Osteophyte formation was not observed in all groups.

2.4. Combined Use of BMSCs and UPAL Gel that Promotes ECM Production in Degenerated IVDs

Next, the present inventor evaluated ECM production in IVDs. In particular, type II collagen is an essential component for IVD function, but in an IVD degeneration process, an increase in the synthesis of such type I collagen is observed [23]. The percentage of type II collagen positive cells was significantly higher in the BMSCs+gel group than in the puncture group and the discectomy group at 4 weeks (p=0.0001, p<0.0001, Tukey-Kramer test), and at 12 weeks, the percentage of type II collagen positive cells was significantly higher in the BMSCs+gel group than in the puncture group, the discectomy group and the gel group (p<0.0001, p<0.0001, p<0.0001, Tukey-Kramer test). Moreover, the gel group also showed a significantly higher percentage of type II collagen positive cells than the discectomy group at 4 weeks (p<0.0001, Tukey-Kramer test), and at 12 weeks, the percentage of type II collagen positive cells was significantly higher in the gel group than in the puncture group and the discectomy group (p=0.0231, p<0.0001, Tukey-Kramer test) (FIG. 5).

On the other hand, the percentage of type I collagen positive cells was significantly lower in the BMSCs+gel group at both 4 weeks and 12 weeks, than in the puncture group (p<0.0001, p<0.0001, Tukey-Kramer test), the discectomy group (p<0.0001, p<0.0001, Tukey-Kramer test) and the gel group (p<0.0001, p<0.0001, Tukey-Kramer test). Moreover, the percentage of type I collagen positive cells was significantly lower in the gel group than in the discectomy group (p<0.0001, p<0.0001, Tukey-Kramer test). At 12 weeks, the percentage of type I collagen positive cells was significantly lower in the gel group than in the puncture group and the discectomy group (p=0.0007, p<0.0001, Tukey-Kramer test). No significant difference was observed between the rabbit BMSCs group and the human BMSCs group by evaluation of both items according to IHC analysis.

2.5. Differentiation of Transplanted BMSCs into NPCs in Degenerated IVDs

Finally, HIF-1α, GLUT-1 and Brachyury positive cells were observed over time, and the mechanism of differentiation of transplanted BMSCs into NPCs in vivo was studied (FIG. 6, a to c). In the intact control group, almost all cells were positive to the three NPC markers at all time points. In the BMSCs+gel group, the HIF-1α, GLUT-1 and Brachyury positive cells were observed at low levels on Day 1, but the number of positive cells increased over time. The percentage of cells positive to the three types of NPC markers to the total number of cells was significantly high on Day 28, compared with Day 1 and Day 7 (p<0.0001, p<0.0001, Student's t-test). In contrast, in the discectomy group, about 20% of cells were positive at all time points (FIG. 6, d to f). Likewise, the percentage of the 3 types of NPC marker positive cells to the number of CFDA-SE positive cells (representing transplanted BMSCs) was significantly high on Day 28, compared with Day 1 and Day 7 (p<0.0001, p<0.0001, Student's t-test) (FIG. 6, g to i).

### 3. Consideration

Since the regeneration ability of IVD is significantly low, tissue repairing is insufficient for a detect generated as a result of discectomy, and it is likely to lead to further IVD degeneration [2]. In the present example, even a single use of UPAL gel suppressed IVD degeneration, compared with after discectomy, but the combined use of BMSCs and UPAL gel exhibited stronger effects of inducing IVD regeneration. Furthermore, in human BMSCs and rabbit BMSCs, IVD regeneration was observed equivalently between the two groups. Several previous studies had also demonstrated the regeneration ability of various BMSCs in degenerated IVDs [18, 24, and 25]. Further, since the UPAL gel exhibits high biocompatibility and sufficient biomechanical properties to support endogenous repair treatment strategies [2], the UPAL gel provides an optimal environment for IVD regeneration after transplantation of BMSCs into degenerated IVD sites and prevention of further degeneration.

In the present example, the gene expression of NPC markers, growth factors and ECMs was significantly increased in the 3D co-culture of NPCs and BMSCs, compared with each 3D monoculture in vitro. These results demonstrate that the co-culture of NPCs and BMSCs leads to differentiation of BMSCs into NPCs, which results in mutual activity between the NPCs and the BMSCs and enhances ECM production in both cell types. These results are consistent with the findings of several other in vitro studies showing that the co-culture of NPCs and BMSCs stimulates the BMSCs to differentiate into an NPC-like phenotype [1, 12, and 26], and support mutual activation between the NPCs and BMSCs via production of growth factors [1, 11, and 27] and up-regulation of ECM synthesis [1, 5, 10, and 26].

Furthermore, an increase in the expression of NPC markers in the embedded BMSCs over time, and similar results showing enhanced ECM production compared with the results after discectomy alone, were observed in an in vivo test. As far as we know, there are no reports regarding the studies of the mechanism of differentiation of BMSCs into NPCs upon in vivo transplantation of alginate and BMSCs. However, a certain study has demonstrated that transplanted MSCs that are embedded in atelocollagen can differentiate into NPCs [28]. Specifically, autologous BMSCs labelled with green fluorescent proteins were transplanted into mature rabbit IVDs, and differentiation of the transplanted cells was measured by IHC analysis. At 48 weeks after the transplantation, it was shown that the transplanted BMSCs were positive to HIF-1α, GLUT-1 and MMP-2, which means that the BMSCs differentiated into cells expressing some of the characteristics of the typical phenotypes of NPCs [28].

In an in vivo test, according to histological analysis, it was further demonstrated that transplantation of gel alone prevents IVD degeneration compared with after discectomy, and that the combined use of BMSCs and gel has stronger effects on prevention of degeneration. These results are consistent with MRI findings suggesting that gel alone preserves a water content in IVD compared with discectomy, but the combination of BMSCs and gel provides stronger effects on the water content preservation. The IHC results further suggest that the combination of BMSCs and gel promotes ECM synthesis in degenerated NP tissues, which is important for appropriate IVD functions and leads to prevention of degenerative progression via a reduction in production of type I collagen. Further, the histological results of BMSCs+gel compared with the puncture group (degenerated IVD that is not attended with discectomy) suggested that BMSC transplantation leads to IVD regeneration. Taken together, these results demonstrate that the transplanted BMSCs differentiate into NPCs over time, resulting in IVD regeneration in vivo.

With regard to AF regeneration, the gel group and the BMSCs+gel group significantly reduced type I collagen production in NPs, whereas filling with gel and BMSCs+gel repaired AF defects caused by puncture. As IVD degeneration is characterized by degradation of NP extracellular matrixes [2], in the present example, preservation/regeneration of NPs was mainly focused.

In intervertebral disc cell therapy, the risk of transplanted cells not being viable in the intervertebral disc and leaking from the injection site has been reported, and it has also been reported that osteophytes may be formed as a result of such cell leakage [5, 29, and 30]. Therefore, in the case of BMSC transplantation after discectomy, a carrier material is needed to prevent such cell leakage. In the present invention, osteophyte formation was not observed in the BMSCs+gel group. The combined use of UPAL gel and BMSCs has an important feature that is immediate curing, and as a result, cell leakage is prevented. In terms of prevention of cell leakage, the combined use of UPAL gel and BMSCs is clinically useful.

Based on the results of the present invention and previous reports [1, 11, 12, and 26-28], the present inventor has conceived that the mechanism of IVD regeneration is as follows.
1) The transplanted BMSCs can be localized in the cavity of the IVD via embedding in UPAL gel, without leaking outside the intervertebral disc.
2) The transplanted BMSCs produce growth factors and ECMs, leading to activation of existing NPCs.
3) The activated NPCs also increase the production of growth factors and ECMs. 4) As a result, the transplanted BMSCs differentiate into NPCs.
5) Furthermore, the BMSCs and the existing NPCs activate each other, resulting in IVD regeneration (FIG. 7).

In the present in vitro experiment, if cell fusion would have taken place between BMSCs and NPCs, separation of the cells would be impossible. Thus, it is unlikely that a cell binding action constitutes a main action mechanism of IVD degeneration. In addition, it was not observed that BMSCs were directly contacted with NPCs in vivo. Therefore, the basic action mechanism may be caused by several important humoral factors that regulate the interaction between growth factors and/or BMSCs, and NPCs.

### REFERENCES

[1] Strassburg S, Richardson S M, Freemont A J, Hoyland J A. Co-culture induces mesenchymal stem cell differentiation and modulation of the degenerate human nucleus pulposus cell phenotype. Regen Med 2010; 5(5): 701-11.
[2] Tsujimoto T, Sudo H, Todoh M, et al. An acellular bioresorbable ultra-purified alginate gel promotes intervertebral disc repair: a preclinical proof-of-concept study. EBioMedicine 2018; 37: 521-34.
[3] Oehme D, Ghosh P, Shimmon S, et al. Mesenchymal progenitor cells combined with pentosan polysulfate mediating disc regeneration at the time of microdiscectomy: a preliminary study in an ovine model. J Neurosurg Spine 2014; 20(6): 657-69.
[4] Noriega D C, Ardura F, Hernandez-Ramajo R, et al. Intervertebral disc repair by allogeneic mesenchymal bone marrow cells: a randomized controlled trial. Transplantation 2017; 101(8): 1945-51.
[5] Naqvi S M, Buckley C T. Differential response of encapsulated nucleus pulposus and bone marrow stem cells in isolation and coculture in alginate and chitosan hydrogels. Tissue Eng Part A 2015; 21(1-2): 288-99.
[6] Sudo H. Exploratory clinical trial on the safety and capability of dMD-001 in lumbar disc herniation. https://upload.umin.ac.jp/cgi-open-bin/icdr_e/ctr_view.cgi?recptno=R000039018. Accessed Dec. 10, 2019.
[7] Yamada K, Sudo H, Iwasaki K, et al. Caspase 3 silencing inhibits biomechanical overload-induced intervertebral disk degeneration. Am J Pathol 2014; 184 (3): 753-64.
[8] Sudo H, Minami A. Caspase 3 as a therapeutic target for regulation of intervertebral disc degeneration in rabbits. Arthritis Rheum 2011; 63(6): 1648-57.
[9] Sato M, Uchida K, Nakajima H, et al. Direct transplantation of mesenchymal stem cells into the knee joints of Hartley strain guinea pigs with spontaneous osteoarthritis. Arthritis Res Ther 2012; 14(1): R31.
[10] Ouyang A, Cerchiari A E, Tang X, et al. Effects of cell type and configuration on anabolic and catabolic activity in 3D co-culture of mesenchymal stem cells and nucleus pulposus cells. J Orthop Res 2017; 35(1): 61-73.
[11] Yamamoto Y, Mochida J, Sakai D, et al. Upregulation of the viability of nucleus pulposus cells by bone marrow-derived stromal cells: significance of direct cell-to-cell contact in coculture system. Spine (Phila Pa 1976) 2004; 29(14): 1508-14.
[12] Richardson S M, Walker R V, Parker S, et al. Intervertebral disc cell-mediated mesenchymal stem cell differentiation. Stem Cells 2006; 24(3): 707-16.
[13] Risbud M V, Schoepfiin Z R, Mwale F, et al. Defining the phenotype of young healthy nucleus pulposus cells: recommendations of the Spine Research Interest Group at the 2014 annual ORS meeting. J Orthop Res 2015; 33(3): 283-93.
[14] Hiyama A, Mochida J, Iwashina T, et al. Transplantation of mesenchymal stem cells in a canine disc degeneration model. J Orthop Res 2008; 26(5): 589-600.
[15] Lei T, Zhang Y, Zhou Q, et al. A novel approach for the annulus needle puncture model of intervertebral disc degeneration in rabbits. Am J Transl Res 2017; 9 (3): 900-9.
[16] Ura K, Sudo H, Iwasaki K, Tsujimoto T, Ukeba D, Iwasaki N. Effects of intradiscal injection of local anesthetics on intervertebral disc degeneration in rabbit degenerated intervertebral disc. J Orthop Res 2019; 37(9): 1963-71.
[17] Qian J, Ge J, Yan Q, Wu C, Yang H, Zou J. Selection of the optimal puncture needle for induction of a rat intervertebral disc degeneration model. Pain Physician 2019; 22(4): 353-60.
[18] Wang H, Zhou Y, Huang B, et al. Utilization of stem cells in alginate for nucleus pulposus tissue engineering. Tissue Eng Part A 2014; 20(5 6): 908-20.
[19] Polzer H, Volkmer E, Saller M M, et al. Long-term detection of fluorescently labeled human mesenchymal stem cell in vitro and in vivo by semi-automated microscopy. Tissue Eng Part C Methods 2012; 18(2): 156-65.
[20] Pfirrmann C W, Metzdorf A, Zanetti M, Hodler J, Boos N. Magnetic resonance classification of lumbar intervertebral disc degeneration. Spine (Phila Pa 1976) 2001; 26(17): 1873-8.
[21] Sakai D, Mochida J, Iwashina T, et al. Regenerative effects of transplanting mesenchymal stem cells embedded in atelocollagen to the degenerated intervertebral disc. Biomaterials 2006; 27(3): 335-45.
[22] Nishimura K, Mochida J. Percutaneous reinsertion of the nucleus pulposus: an experimental study. Spine (Phila Pa 1976) 1998; 23(14): 1531-8.
[23] Le Maitre C L, Pockert A, Buttle D J, Freemont A J, Hoyland J A. Matrix synthesis and degradation in human intervertebral disc degeneration. Biochem Soc Trans 2007; 35(Pt 4): 652-5.
[24] Wang F, Nan L P, Zhou S F, et al. Injectable hydrogel combined with nucleus pulposus-derived mesenchymal stem cells for the treatment of degenerative intervertebral disc in rats. Stem Cells Int 2019: 8496025.
[25] Omlor G W, Lorenz S, Nerlich A G, Guehring T, Richter W. Disc cell therapy with bone-marrow-derived autologous mesenchymal stromal cells in a large porcine disc degeneration model. Eur Spine J 2018; 27(10): 2639-49.
[26] Risbud M V, Albert T J, Guttapalli A, et al. Differentiation of mesenchymal stem cells towards a nucleus pulposus-like phenotype in vitro: implications for cell-based transplantation therapy. Spine (Phila Pa 1976) 2004; 29(23): 2627-32.
[27] Yang S H, Wu C C, Shih T T, Sun Y H, Lin F H. In vitro study on interaction between human nucleus pulposus cells and mesenchymal stem cells through paracrine stimulation. Spine (Phila Pa 1976) 2008; 33(18): 1951-7.
[28] Sakai D, Mochida J, Iwashina T, et al. Differentiation of mesenchymal stem cells transplanted to a rabbit degenerative disc model: potential and limitations for stem cell therapy in disc regeneration. Spine (Phila Pa 1976) 2005; 30(21): 2379-87.
[29] Li Y Y, Diao H J, Chik T K, et al. Delivering mesenchymal stem cells in collagen microsphere carriers to rabbit degenerative disc: reduced risk of osteophyte formation. Tissue Eng Part A 2014; 20(9 10): 1379-91.
[30] Vadala G, Sowa G, Hubert M, Gilbertson L G, Denaro V, Kang J D. Mesenchymal stem cells injection in degenerated intervertebral disc: cell leakage may induce osteophyte formation. J Tissue Eng Regen Med 2012; 6(5): 348-55.
[31] Dominici M, Le Blanc K, Mueller I, et al. Minimal criteria for defining multipotent mesenchymal stromal cells. The International Society for Cellular Therapy position statement. Cytotherapy 2006; 8(4): 315-7.
[32] Raynaud C M, Maleki M, Lis R, et al. Comprehensive characterization of mesenchymal stem cells from human placenta and fetal membrane and their response to osteoactivin stimulation. Stem Cells Int 2012; 2012: 658356.
[33] Serigano K, Sakai D, Hiyama A, Tamura F, Tanaka M, Mochida J. Effect of cell number on mesenchymal stem cell transplantation in a canine disc degeneration model. J Orthop Res 2010; 28: 1267-75.

### Example 2

Changes in gene expression due to co-culture (in vitro) of nucleus pulposus cells (NPCs) and high purity mesenchymal stem cells (RECs)

### 1. Materials and Methods

### 1.1. Human Healthy Intervertebral Disc Nucleus Pulposus Cells (NPCs)

As human healthy intervertebral disc nucleus pulposus cells (NPCs), the cells that were obtained during anterior spinal fusion surgery in young patients (15.3±3.3 years old) with adolescent idiopathic scoliosis at Hokkaido University Hospital were used.

### 1.2. Preparation of High Purity Mesenchymal Stem Cells Having High Uniformity

### (1) Measurement of CV Value of Forward Scattered Light (FSC) According to Flow Cytometry

A plurality of REC clones that had previously been prepared according to a known method (WO2016/17795) were used to measure the CV value of a forward scattered light by flow cytometry.

The FSC in flow cytometry is proportional to the surface area or size of a cell. In the present example, the CV value of FSC was used as an indicator to evaluate a variation in the cell size.
(a) PI staining was performed on individual REC clones, and dead cells were excluded from the analysis subject by setting a gate on a PI-negative live cell population.
(b) The PI-negative live cell population was expanded in the FSC/SSC cytograms, and a gate (P1) was set on a main cell population to exclude contaminants and noises from the analysis subject.
(c) The cell population in the P1 gate was expanded in the FSC histogram, a marker (M1) was set, and the CV value was measured.

### (2) Evaluation of Cell Proliferation Ability and Adipose Differentiation Ability

In order to evaluate cell proliferation ability, 1×10⁵ REC cells were seeded on a 100 mm culture dish, and were then cultured for 5 days in an environment of 37° C. and 5% CO2, and thereafter, the number of cells and an average cell size were measured using a cell counter.

The used culture solution was a DMEM medium (FUJIFILM Wako Pure Chemical Corporation) supplemented with FBS, basic FGF, Hepes and Penicillin-Streptomycin.

In addition, in order to evaluate adipose differentiation ability, 5×10⁴ REC cells were seeded on a 24-well plate, and were then cultured for 2 days in an environment of 37° C. and 5% CO2, followed by the replacement of the medium with an adipose differentiation induction medium, and the cells were further cultured for 14 days. At 14 days after the cultures, Oil red 0 staining was performed, and the lipid droplet area was then calculated according to image analysis. Besides, the adipose differentiation induction medium was a medium prepared by adding Dexamethasone, Indomethacin and IBMX to the above-described culture medium.

### (3) Comprehensive Analysis

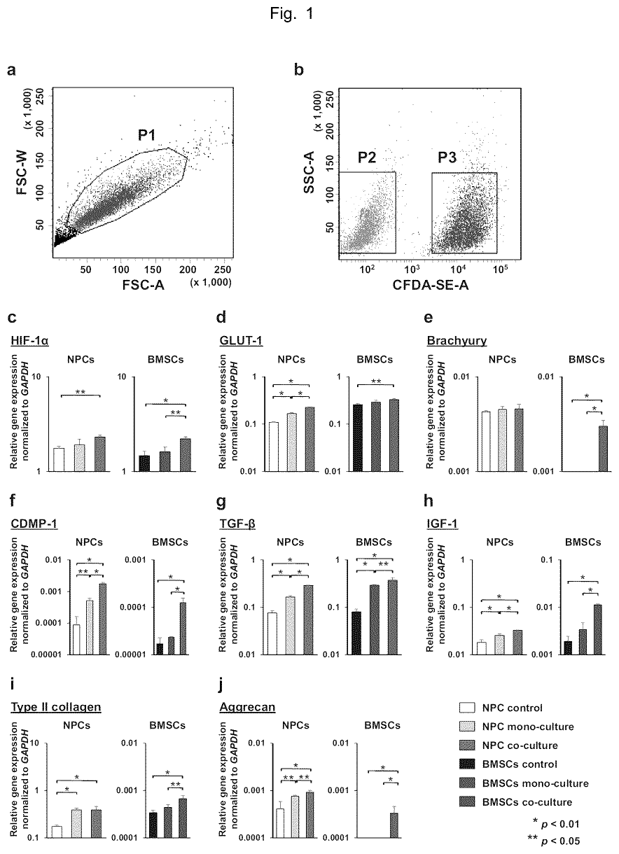

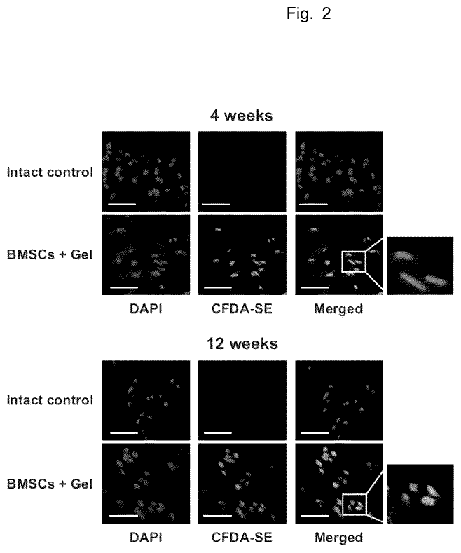

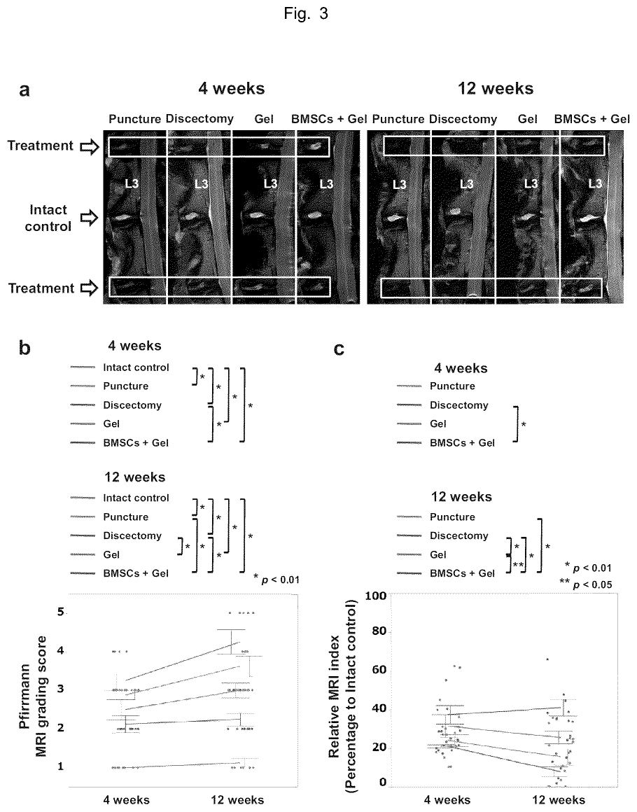

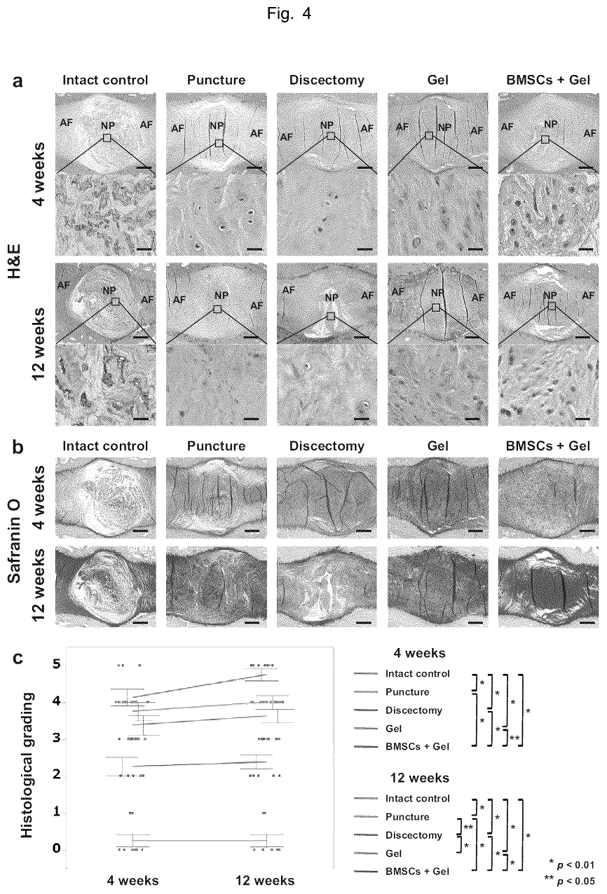

Regarding a plurality of several REC clones, the CV value of FSC, cell proliferation percentage (proliferation ability), average cell size, and adipose differentiation ability were examined. As a result, it was found that REC clones with a low CV value of FSC (CV=35% or less) had high proliferation ability and high differentiation ability and also had a small average cell size (average cell size: 20 lam or less) (FIG. 8).

On the other hand, it was found that REC clones with a high CV value of FSC (CV=35% or more) had low proliferation ability and low differentiation ability and also had a large average cell size.

The average proliferation rate of clones with a CV value of 30% to 35% was 6.4, the average proliferation rate of clones with a CV value of 25% to 30% was 9.2, and the average proliferation rate of clones with a CV value of 25% or less was 15.1. The average proliferation rate of clones with an average cell size of 18 lam to 20 lam was 7.0, and the average proliferation rate of clones with an average cell size of 16 lam to 18 lam was 12.7. The average proliferation rate of clones with an average cell size of 16 lam or less was 21.3

Hereafter, in the present example, REC clones having a low CV value of FSC (CV=25% or less) were used.

### 1.3. Preparation of UPAL Composition Comprising RECs

NPCs and fluorescently-labelled high purity mesenchymal cells (RECs) were each divided into a single group and a mixed group, and were then subjected to co-culture or monoculture in the 3D gel of ultra-pure alginic acid (UPAL: provided by Mochida Pharmaceutical Co., Ltd.). Before the culture (day 0) and 7 days after the culture, the 3D gel was dissolved, and the cells were separated into the following cell groups using a cell sorter.
(a) NPC control group (day 0)
(b) NPC monoculture group
(c) NPC co-culture group
(d) REC control group (day 0)
(e) REC monoculture group
(f) REC co-culture group

The cells of the above 6 groups were subjected to quantitative PCR analysis (qRT-PCR), and the expression levels of the following genes were measured.

Markers for nucleus pulposus cells
HIF-1α
GLUT-1
Brachyury
   Growth factors
CDMP-1
TGF-β
IGF-1
   Extracellular matrixes
Type II collagen
Aggrecan

The expression level of each gene was measured according to the method applied in Example 1.

### 2. Results

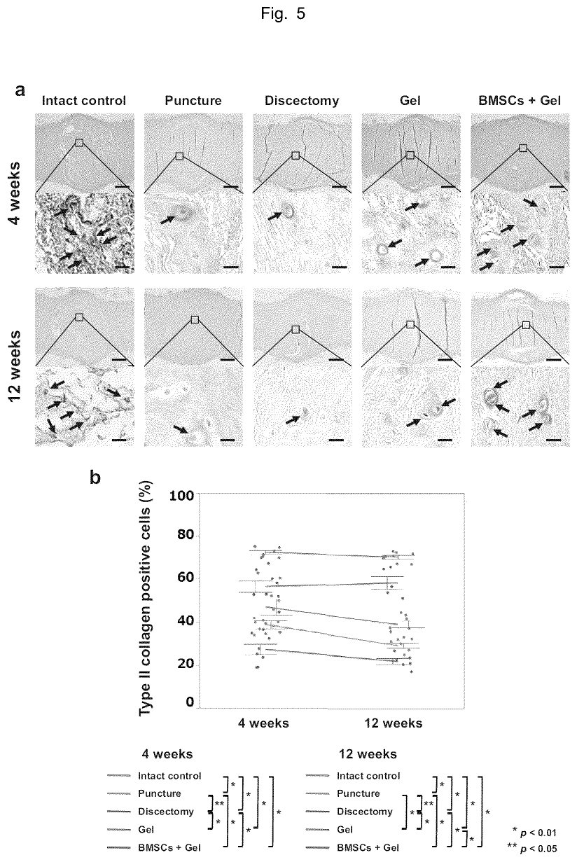

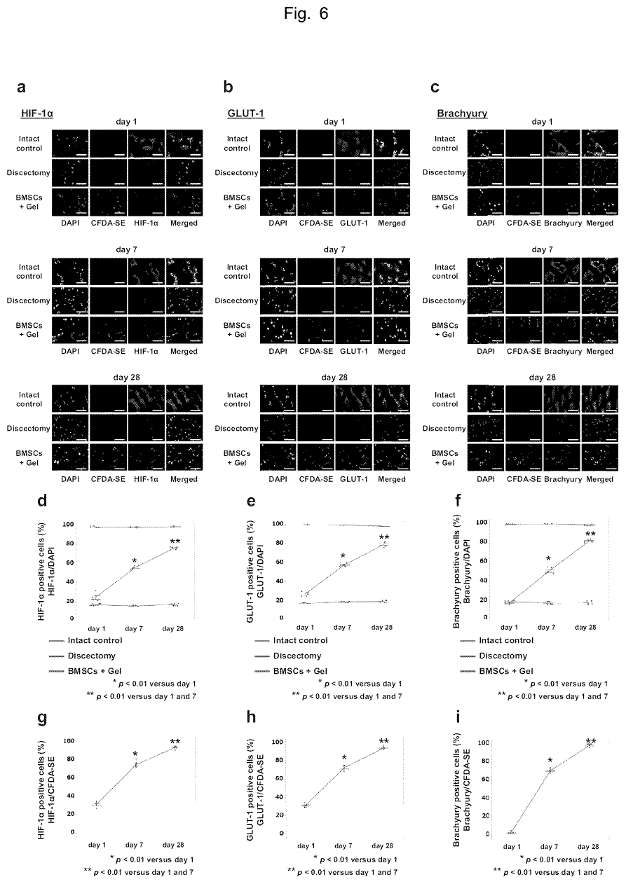

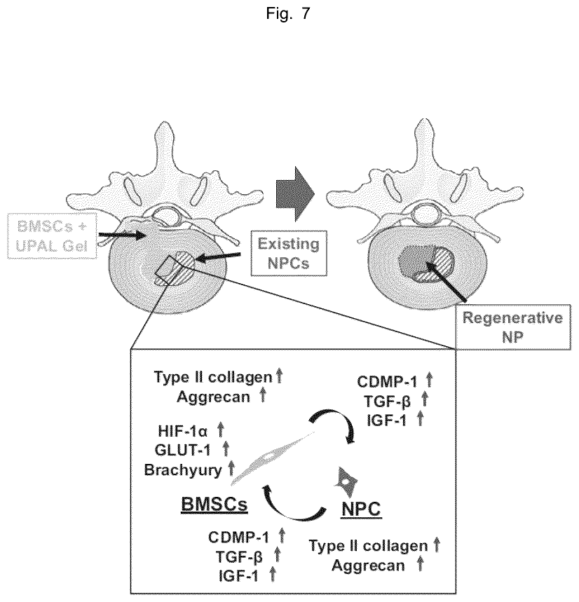

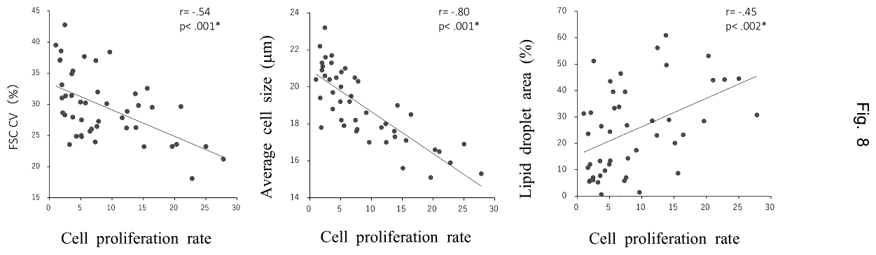

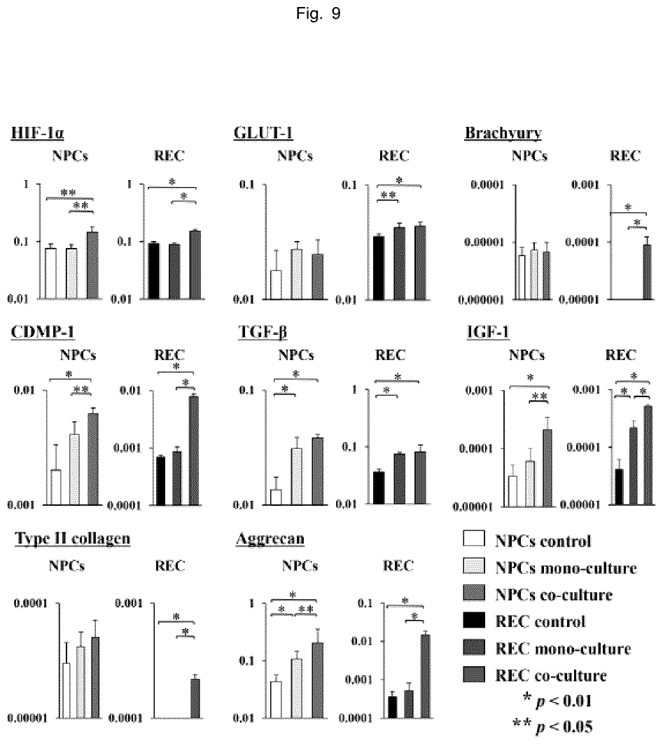

When RECs were co-cultured with nucleus pulposus cells, differentiation indicators (HIF-1α, GLUT-1 and Brachyury) were increased in both the nucleus pulposus cells and the mesenchymal stem cells, compared with the case of monoculture (FIG. 9).

Similar results were obtained regarding growth factors (CDMP-1, TGFβ and IGF-1) and extracellular matrixes (type II collagen and aggrecan).

It is suggested that nucleus pulposus cells interact with highly purified mesenchymal stem cells, and become activated, and that the mesenchymal stem cells differentiate into nucleus pulposus cells.

### Example 3

Effect of Promoting Regeneration in Sheep Intervertebral Disc Degeneration Models (MRI Analysis) and Histological Analysis

### 1. Materials and Methods

Seven sheep (40 to 50 kg) were treated in accordance with GLP.

The intervertebral disc L1/L2 (an intervertebral disc located between the first and second lumbar vertebrae), the intervertebral disc L2/L3 (an intervertebral disc located between the second and third lumbar vertebrae), the intervertebral disc L3/L4 (an intervertebral disc located between the third and fourth lumbar vertebrae), and the intervertebral disc L4/L5 (an intervertebral disc located between the fourth and fifth lumbar vertebrae) were divided into the following 4 groups

Control group (n=6): Non-treated group

discectomy group (n=6): Only excision of nucleus pulposus tissues

Gel group (n=8): After excision of nucleus pulposus tissues, embedding of alginate gel

RECs+gel group (n=8): After excision of nucleus pulposus tissues embedding of RECs and alginate gel

With the exception of the non-treated group, 20 mg of nucleus pulposus tissues were removed under anesthesia from the intervertebral disc of each group, so as to cause severe intervertebral disc degeneration.

At 4 weeks after the initial excision of nucleus pulposus tissues, 70 mg of nucleus pulposus tissues were further excised under anesthesia, and thereafter, RECs (the RECs prepared in Example 2, Section 1.2) suspended in UPAL at a final cell concentration of 1×10⁶ cell/ml (100 μl) were then injected into the voids in the intervertebral disc.

At 4 weeks after the embedding surgery, the sheep were euthanized.

For the purpose of quantitative evaluation of degeneration of the treated intervertebral discs, the treated intervertebral discs were analyzed using 3 Tesla magnetic resonance imaging (MRI), and thereafter, the intervertebral discs were subjected to histological evaluation by H & E staining and safranin-O staining, or to evaluation of the expression of type II collagen according to immunohistochemical evaluation.

All data are shown in the form of a mean value±standard error, and differences among individual groups were tested by one-way analysis of variance (ANOVA) and a Tukey-Kramer post hoc test.

### 2. Results

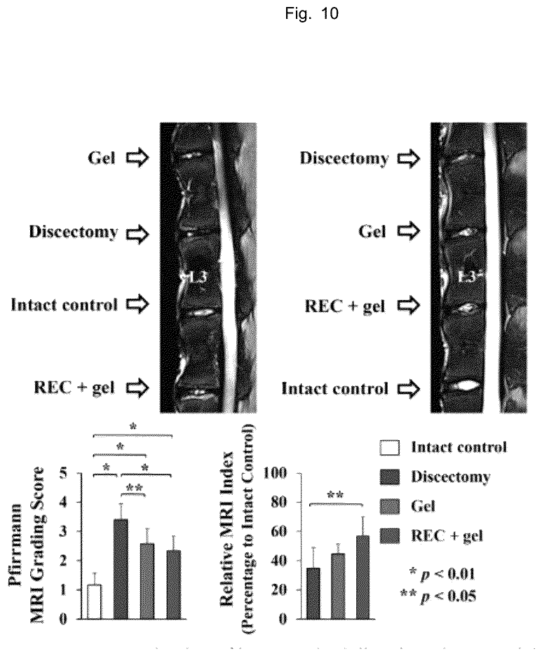

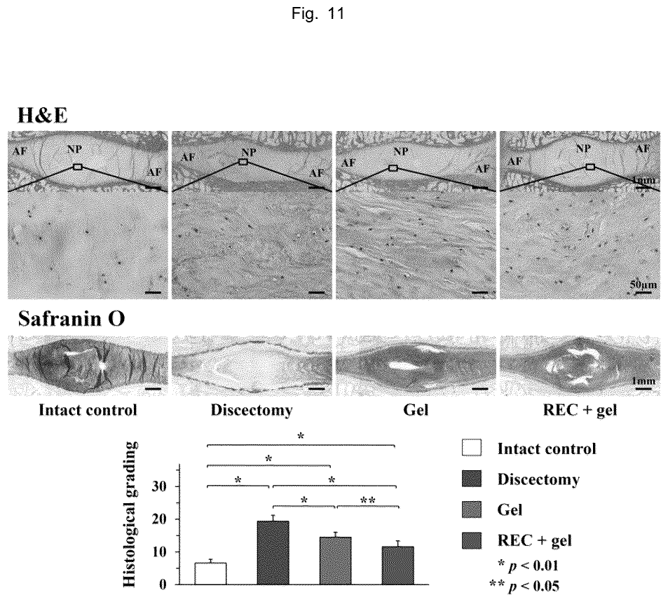

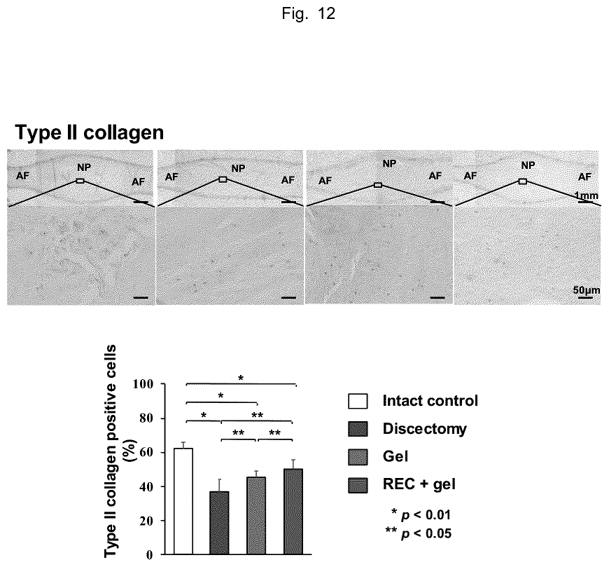

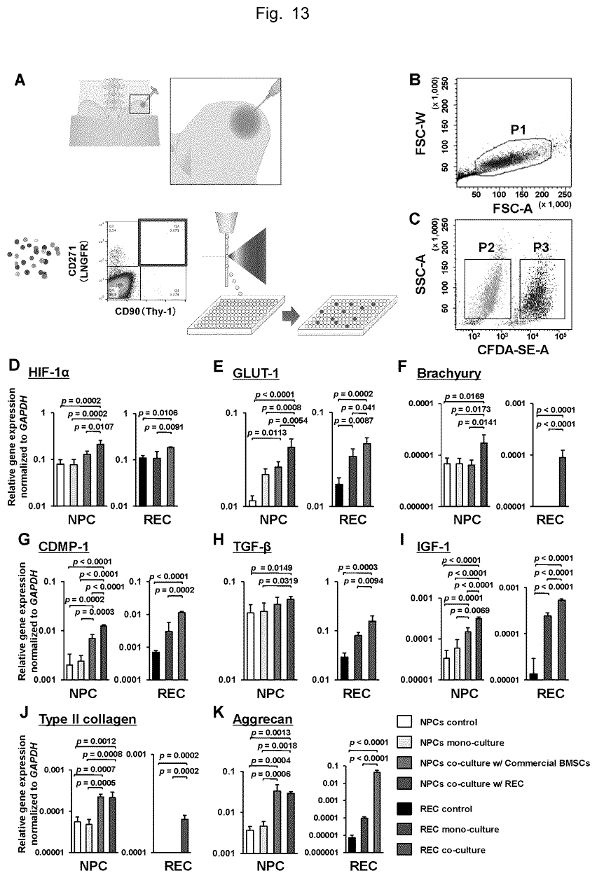

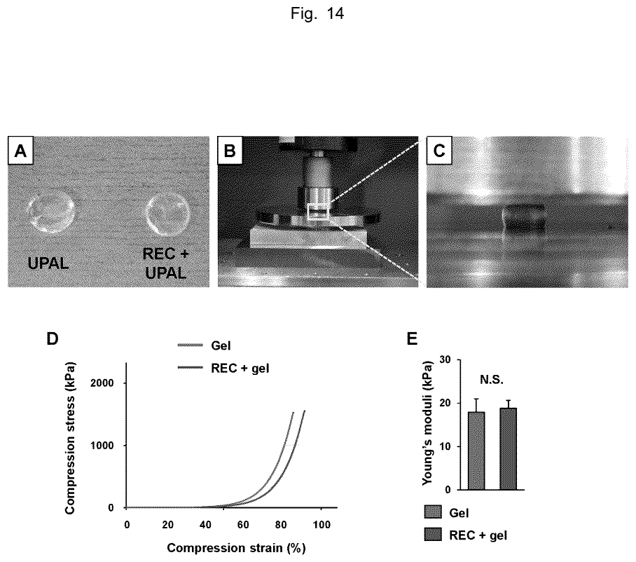

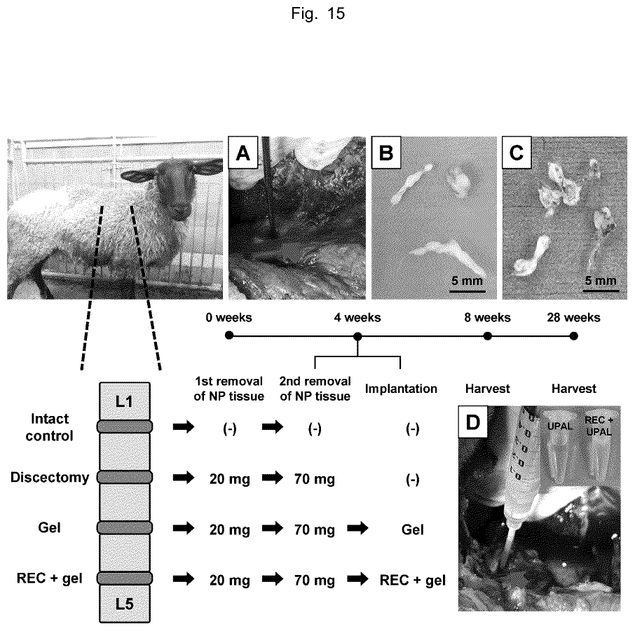

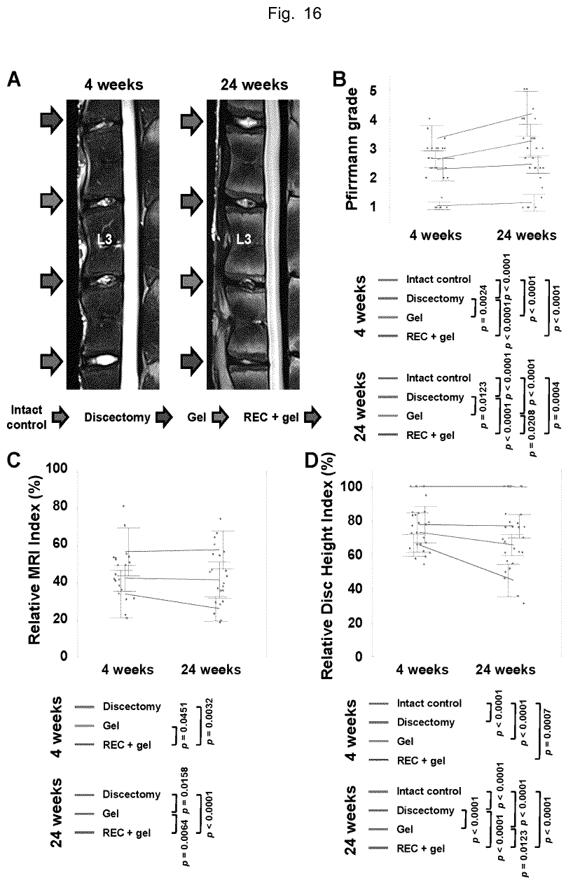

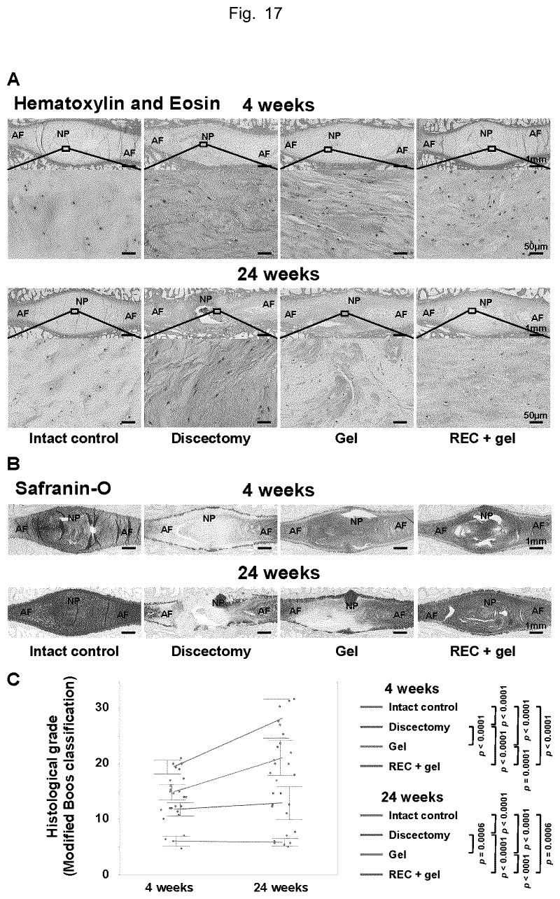

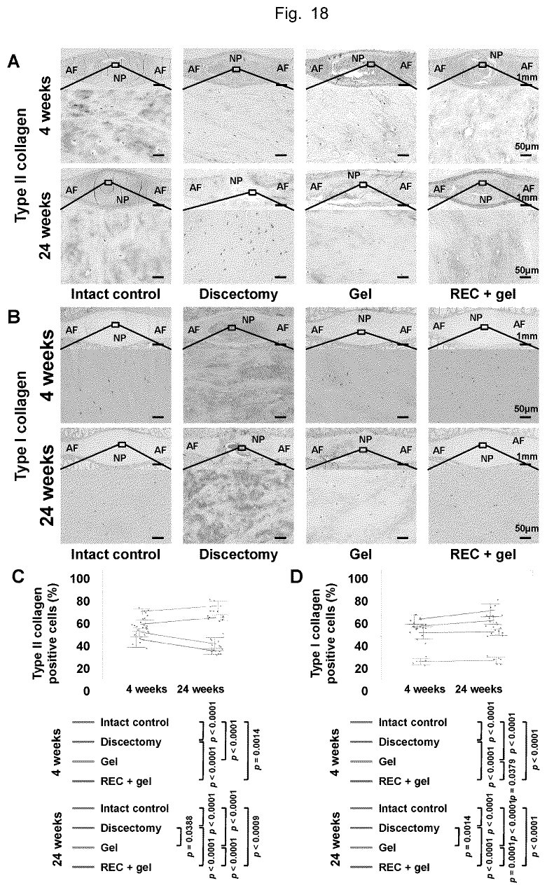

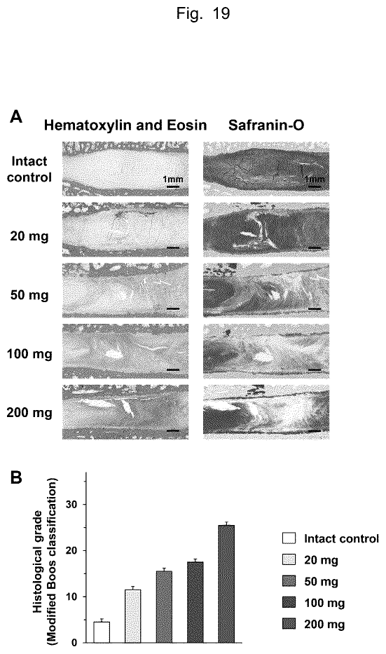

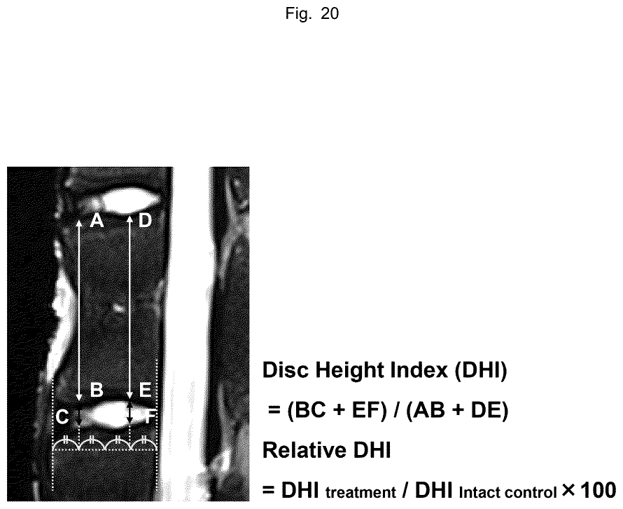

Gel and RECs+gel were embedded into the affected areas of sheep intervertebral discs, and at 4 weeks after the embedding, degeneration of the intervertebral discs was evaluated according to MRI. As a result, according to Pfirrmann classification, degeneration of the intervertebral discs was significantly low in the gel group and the RECs+gel group, compared with the non-treated group (discectomy group). In addition, the MRI index was significantly high in the RECs+gel group, compared with the non-treated group (discectomy group) (FIG. 10). The histological degeneration score according to histological staining (Boos classification) was significantly low in the gel group and the RECs+gel groups, compared with the non-treated group (discectomy group). Moreover, when the gel group was compared with the RECs+gel group, the histological degeneration score was significantly lower in the RECs+gel group than in the gel group (FIG. 11). According to immunohistochemical analysis, the percentage of type II collagen positive cells was significantly higher in the RECs+gel group than in the non-treated group (discectomy group) and the gel group (FIG. 12).

The embedding of UPAL gel comprising RECs suppressed IVD degeneration after discectomy in highly degenerated IVDs, and the rate of type II collagen positive cells was significantly high in the RECs+gel group. Accordingly, it was demonstrated that RECs used in combination with UPAL gel promote spontaneous IVD regeneration. Furthermore, the present inventor has demonstrated that the combined use of RECs and UPAL gel promotes endogenous NPC activation, as well as production of growth factors and ECMs, and the combined use promotes differentiation into NPCs and enhances IVD regeneration.

### Example 4

In the present example, it was demonstrated that, in sheep models showing severe IVD degeneration, a combination of bone marrow-derived high purity mesenchymal stem cells (RECs) and UPAL gel enhances IVD regeneration in situ after discectomy (until 24 weeks). Further, in the present example, healthy human NPCs and RECs were co-cultured in a 3D system in UPAL gel, and the mechanism that serves as a basis of IVD regeneration was evaluated. Moreover, the co-culture of NPCs and commercially available BMSCs and the co-culture of NPCs and REC cells were performed, and the RECs were compared with the commercially available BMSCs, in terms of their effects on the NPCs. The findings of the present inventors suggest the clinical applicability of a combination of RECs and in situ curing gel for the treatment of hernias in degenerative human IVDs.

### 1. Materials and Methods

### 1.1. Test Design

The test design in the present example was carried out as follows.
(i) Studies of the mechanism of IVD regeneration via the 3D co-culture of human NPCs and RECs in vitro.
(ii) Comparison between the effects of RECs on NPCs and the effects of commercially available human BMSCs on NPCs, via the 3D co-culture of the RECs or the commercially available human BMSCs, and the NPCs.
(iii) Measurement of the mechanical properties of UPAL and RECs+UPAL gel.
(iv) Evaluation of the regeneration ability of severely degenerated IVDs after discectomy in vivo.
(v) Evaluation of tumor formation in transplanted IVDs.

The present inventors have assumed that transplantation of RECs embedded in UPAL gel after discectomy would promote spontaneous IVD regeneration.

The use of healthy human IVDs was approved by the Ethics Committee of the Hokkaido University Graduate School of Medicine. Animal experimental procedures were approved by the Institutional Animal Care and Use Committee of Hokkaido University and Hamri Corporation (Ibaraki, Japan), and were performed according to the guidelines recommended by these committees.

Healthy human NPCs and RECs were co-cultured in a 3D system in UPAL gel, and the mechanism that serves as a basis of IVD regeneration was evaluated. On Day 0 and Day 7 after the culture, the expression of NPC markers (including HIF-1α, GLUT-1 and brachyury), growth factors (including CDMP-1, TGF-β and IGF-1), and ECM components (including type II collagen and aggrecan) was analyzed in all cell types using qRT-PCR (n=4) (2, 9, 30, 31). Furthermore, NPCs and commercially available BMSCs were co-cultured, and the effects of RECs and commercially available BMSCs on NPCs were compared with each other.

Subsequently, an unconstrained compression test was used to evaluate the mechanical properties of the UPAL and the RECs+UPAL gel. Disc-shaped gel was compressed at a constant rate of 0.5 mm/min, and Young's modulus was calculated (n=4) (3, 15, 32, and 33). In the animal experiment, 14 sheep (56 IVDs in total) were divided into the following 4 groups. The results obtained for 4 weeks are also described in Example 3.

Intact control group (4 weeks, n=6; 24 weeks, n=6): Non-treated group

Discectomy group (4 weeks, n=6; 24 weeks, n=6): Only excision of nucleus pulposus tissues, and this group is also referred to as an “intervertebral disc resection group”

Gel group (4 weeks, n=8; 24 weeks, n=8): After excision of nucleus pulposus tissues, embedding of alginate gel

RECs+gel group (4 weeks, n=8; 24 weeks, n=8): After excision of nucleus pulposus tissues, embedding of RECs and alginate gel

In order to produce a severely degenerated IVD, the present inventors removed 20 mg of NP tissues from the treated IVD. The present inventors further removed 70 mg of NP tissues from the degenerated IVD at 4 weeks after the initial surgery. In a preliminary experiment, since the removal of more than 20 mg of NP tissues from the sheep model caused IVD degeneration 4 weeks later, the initial amount of tissues to be removed was set at 20 mg (FIG. 19). The second amount of tissues to be moved was set at 70 mg, based on the ratio between the amount of normal IVDs removed and the amount of degenerated IVDs removed in previous experiments with the rabbit models (2 and 3). After the second discectomy, a solution containing UPAL or a combination of RECs and UPAL was transplanted into the void of the intervertebral disc. Sheep were euthanized 4 weeks (Example 3) and 24 weeks after the transplantation. In order to evaluate IVD degeneration, using 3.0-T MRI (2, 3, 15, 37, and 38), the IVDs were analyzed qualitatively. Subsequently, the IVDs were stained with H & E and with safranin-O for histological analysis, and the levels of type II and type I collagens were evaluated by IHC for the analysis of ECM components. Finally, tumorigenesis analysis was performed on tissue specimens (2, 3, and 15).

### 1.2. Statistical Analysis

The sample size for the quantitative data was determined according to power analysis at a levels of 0.05 and 0.8 powers, using a Tukey-Kramer test. Statistical analyses were performed using JMP Pro version 14.0 software (SAS Institute, Cary, NC, USA), and values were considered to be significant in the case of p<0.05. All data are shown in the form of a mean±SD value. One-way analysis of variance (ANOVA) and a Tukey-Kramer post-test were used for multiple group comparison. A Student's t-test or a Mann-Whitney's U-test and a Welch's test were used for comparison between two groups. The present inventors randomized the samples and were blinded to the samples to be tested.

### 1.3. 3D Co-Culture of RECs or Commercially Available Human BMSCs and Human NPCs In Vitro

### Preparation of RECs

In the present test, frozen GMP-compliant RECs provided by Japan Tissue Engineering Co., Ltd. were used. In a comparative analysis of RECs and commercially available human BMSCs, three clones of RECs, namely, REC-02 prototype #003-P6-191220, REC-02 HLWF-1-P6-210114, and REC-02 QRKF-1-P6-210210 were prepared at passage 6. The cells were thawed in a warm bath at 37° C. before 3D culture.

### Preparation of Commercially Available Human BMSCs

Commercially available human BMSCs (hMSC-BM; PromoCell, Heidelberg, Germany; C-12974, Lot No.: 412Z022.4) were obtained according to the previous report (2). The properties of these cells belonging to osteogenic, chondrogenic and adipogenic lineages, the viability of the cells after thawing, cell cycle, undifferentiated state, and multi-differentiation ability were tested. The BMSCs were cultured using a complete culture medium, namely, Dulbecco's Modified Eagle Medium (low glucose level; 2 mg/mL) containing L-glutamine and phenol red (DMEM; FUJIFILM Wako Pure Chemical Corporation, Osaka, Japan), which was supplemented with 20% HyClone fetal bovine serum (FBS; Cytiva, Tokyo, Japan), 1% penicillin/streptomycin, 1.25 mg/mL fungizone (Life Technologies, Thermo Fisher Scientific, Waltham, M A, and Scientific, Waltham, MA, USA), 1% HEPES (Life Technologies, Thermo Fisher Scientific), and 0.1% bFGF (Kaken Pharmaceutical Co., Ltd., Tokyo, Japan), according to the manufacturer's instructions. The medium was exchanged twice a week, and the BMSCs from the fourth passage process were used (a total of six passages that were the same as for REC).

### Preparation of UPAL Gel and 3D Culture

UPAL gel (Mochida Pharmaceutical Co. Ltd., Tokyo, Japan) was used as an alginate scaffold for 3D culture (2 and 3).

A 2% (w/v) UPAL solution dissolved in phosphate buffered saline (PBS; FUJIFILM Wako Pure Chemical Industries) was prepared, and was then gelled using a CaCl₂ solution (102 mM). RECs or commercially available BMSCs were mixed with the UPAL solution at a final cell concentration of 1×10⁶ cells/mL (2, 31, and 49). The cell-UPAL mixed solution was pipetted into a 102 mM CaCl₂ solution for gelling, using a 22-gauge needle. The two types of gels obtained in the form of beads were cultured with a medium for 7 days in a humidified environment (20% 02, 5% CO2, and 37° C.). The medium was exchanged every 3 days. In order to recover the cells at 7 days after the 3D culture, as previously reported (2, 3, and 35), the cells were lysed using 55 mM sodium citrate, and were then centrifuged (110×g for 10 min at 4° C.). Thereafter, the cells were recovered from the gel beads. Furthermore, as mentioned above, RECs and commercially available BMSCs prepared by planar culture (2D culture) for 7 days under normal conditions were used as control groups (i.e. the following 4 groups were established as experimental groups: 1) 2D culture RECs, 2) 3D culture RECs, 3) 2D culture BMSCs, and 4) 3D culture BMSCs).

### Analysis of Cell Proliferation Ability

After 7 days of the 2D or 3D culture, the obtained 4 types of cells were seeded again for secondary culture. The cells were recovered when they reached confluence, and the cell proliferation ability was then evaluated based on the doubling time (Td), which is defined as a time required for the number of cells to double. The Td was calculated according to the following equation:

Td=(t2−t1)×ln(2)/ln(N2/N1),

wherein N2 and N1 each represent the number of cells at the times t2 and t1.

### Flow Cytometric Analysis

PBS (containing 2% FBS) was added to each of the 4 types of cells to prepare cell suspensions at a final density of 1×10⁶ cells/mL. Subsequently, the cells were stained with an anti-CD90 antibody (PE anti-human CD90 (Thy1), BioLegend, San Diego, CA, USA) and an anti-CD45 antibody (FITC anti-human CD45, Beckman Coulter, Brea, CA, USA). Staining was also performed with a mouse IgG1 antibody (mouse IgG1-PE, BioLegend; mouse IgG1-FITC, Beckman Coulter) as a negative control. Dead cells were detected using propidium iodide (PI). Flow cytometric analysis was performed using CytoFLEX System (Beckman Coulter), and cell viability and the uniformity and positivity of each cell surface antigen were evaluated. Data were analyzed using FlowJo software (Becton Dickinson, Franklin Lakes, NJ, USA).

### Preparation of Healthy Human NPCs

Human NP samples were obtained from 9 patients (mean age±SD, 15.3±3.3 years old) who underwent anterior spinal fusion for adolescent idiopathic scoliosis at Hokkaido University Hospital. Informed consent was obtained from the participants who participated in the present study, and a consent form was drawn up and kept on file. The samples were obtained during surgical operations. All IVDs were analyzed before the surgery via MRI, and degenerative changes were evaluated using the Pfirrmann classification (36). All IVDs were classified into grade 1, suggesting that all samples were non-degenerated IVDs.

The cells were isolated from human NP samples, and were then cultured as previously reported (2 and 3). First, each gel-like NP was separated from AF under a dissecting microscope, and the tissue specimen was placed in a medium. The prepared product was washed twice by centrifugation (1,000 rpm, 3 minutes), and was then resuspended in a medium supplemented with 0.25% collagenase. For cell isolation, the prepared product was incubated in a shaking incubator (37° C., 4 hours), followed by centrifugation (1,000 rpm, 3 minutes) twice. The cells separated from the matrix were placed in a 10 cm tissue culture dish, and were then incubated as described above. The medium was exchanged twice a week, and NPCs from the fourth passage were used.

### Preparation of RECs and Commercially Available Human BMSCs

The above-described RECs (REC-02_prototype #003-P6-191220) and commercially available human BMSCs were also used in the 3D co-culture. The 2 types of cells were cultured by the same methods as those for the 2D cultures described above, according to the manufacturer's instructions. The medium was exchanged twice a week. Both types of cells derived from the second passage (RECs: 8 passages in total; BMSC: 4 passages in total) were used.

### 3D Co-Culture and Monoculture

The present inventors prepared a 2% UPAL solution, and used a CaCl₂ solution (102 mM) for gelling, as previously reported (2 and 3). Before 3D culture, RECs and BMSCs were fluorescently labelled with 20 mM CFDA-SE (CFDA-SE cell growth assay kit; BIO RAD, Hercules, CA, USA) with reference to the manufacturer's manuals (2, 15, and 29). Thereafter, the labelled cells and the non-labelled NPCs were mixed into a UPAL solution at the same ratio (1×10⁶ cells/mL for each type of cells) (2, 31, and 49) as in, to obtain a final cell concentration of 2×10⁶ cells/mL. The cell-UPAL mixed solution was added into a 102 mM CaCl₂ solution using a 22-gauge needle, and was then gelled. The obtained gel was cultured under hypoxic conditions (5% 02 and 5% CO2) (2 and 49) for 7 days. Further, the 3 types of cells were mixed separately into a UPAL solution at a concentration of 1×10⁶ cells/mL. The cell concentration was selected on the basis of the results of the previous report (2). After gelling, the gel beads were cultured under hypoxic conditions by the same method as described above, and the following experimental groups were used (n=4 per group):
(a) Monocultured NPCs,
(b) Monocultured BMSCs,
(c) Monocultured RECs,
(d) Co-cultured NPCs+BMSCs, and
(e) Co-cultured NPCs+RECs.

On the first day and at 7 days after the culture, all gel beads were dissolved in 55 mM sodium citrate, and the gel was separated from the cells. In the co-culture groups, the recovered cells were sorted using a BD FACSAria III High speed cell sorter with Diva software version 7.0 (BD Biosciences, San Jose, CA, USA) (2 and 31). After the removal of residues and dead cells, the fluorescent cells at 530 nm were selected as RECs and BMSCs, and non-fluorescent cells were selected as NPCs.

RNA Extraction and qRT-PCR

The recovered 10 types of cells (i.e. control NPCs, monocultured NPCs, NPCs co-cultured with commercially available BMSCs, NPCs co-cultured with RECs, control BMSCs, monocultured BMSCs, co-cultured BMSCs, control RECs, monocultured RECs, and co-cultured RECs) were dissolved in 1 mL of TRIzol (registered trademark) (Invitrogen, Thermo Fisher Scientific), and total RNA was then extracted from the samples using the RNeasy Mini kit (Qiagen, Valencia, CA, USA). Real-time qRT-PCR was performed using the TaqMan (registered trademark) Gene Expression Assay (Applied Biosystems, Thermo Fisher Scientific) (Table 3). Cycle threshold (Ct) values were obtained for individual samples. Furthermore, the relative mRNA expression levels of individual target genes, NPC markers, growth factors, and ECM components were calculated via a 2-ΔCt method (2). The expression levels were standardized by the expression level of the housekeeping gene GAPDH (2 and 31).

### 1.4. Elastic Analysis of Gels Using Unconfined Compression Test

The mechanical properties of the UPAL and the RECs-UPAL gel were evaluated using an unconfined compression test. Two types of disc-shaped gels with a diameter of 4.5 mm and a thickness of 2 mm were prepared (FIG. 14(a)). The samples were placed in a tensile-compression machine (Autograph AG-X; Shimadzu Corporation, Kyoto, Japan), and were compressed at a constant rate of 0.5 mm/min using a 100 N load cell (FIGS. 14, B and C) until the gel was crushed (3 and 15). The Young's modulus was calculated using an approximate straight line between compression values of 10% to 20% (n=4 gels/group) (FIG. 14E) (3, 15, 32, and 33), based on the obtained stress-strain curve (FIG. 14D).

1.5. In Vivo Test Using Sheep Models with Degenerated IVDs

In the present example, all procedures including the sheep models were performed in a pharmaceutical GLP-compliant laboratory (Hamri Co., Ltd.) (3). Fourteen male, Suffolk sheep (2 years old, a body weight of 40 to 60 kg) were used for qualitative analysis of IVD degeneration (3). A total of 56 IVDs were randomly assigned to intact control (4 weeks, n=6; 24 weeks, n=6), discectomy (4 weeks, n=6; 24 weeks, n=6), gel (4 weeks, n=8; 24 weeks, n=8), and RECs+gel (4 weeks, n=8; 24 weeks, n=8). First, the NP tissues were surgically removed to create a severely degenerated IVD model. Anesthesia was induced by intramuscular injection of a 4: 1 mixture of ketamine (0.2 mg/kg) and xylazine (20 mg/kg) at a rate of 0.5 mL/kg, and then, the maintenance of anesthesia was achieved by inhalation anesthesia (isoflurane). The surgery was performed via a right lateral retroperitoneal approach, and vertebral bodies and IVDs from L1 to L5 were exposed. A solid trabecular screw (ZIMMER BIOMET, Warsaw, IN, USA) was inserted into the L2 vertebral body as a vertebra marker. In the discectomy group, the gel group and the RECs+gel group, 20 mg of NP tissues were removed after AF incision (5×3 mm), so as to induce IVD degeneration (FIGS. 15, A and B) (3 and 8).

### Removal of Degenerated NP Tissues and Transplantation

At 4 weeks after the initial surgery, the vertebral bodies and IVDs were exposed using the same approach as described above. Further, in order to create IVD cavities after the AF incision as described above, the present inventors extracted 70 mg of NP tissues from the degenerated IVDs that were obtained from the three treatment groups (FIG. 15C). After discectomy, in the gel group, 110 to 120 μL of a 2% UPAL solution was transplanted into the voids in the intervertebral disc, and in the RECs+gel group, 110 to 120 μL of a mixture of RECs and a UPAL solution (final concentration: 1×10⁶ cells/mL) was transplanted into the voids in the intervertebral disc (FIG. 15D) (2). Immediately after the transplantation, a 102 mM CaCl₂ solution was injected into the surface of the mixture, and 5 minutes later, gelling was confirmed. At 4 weeks and 24 weeks after the transplantation, the sheep were euthanized with pentobarbital, and the lumbar spinal column was removed as a single mass (3).

### MRI

T2-weighted mid-sagittal section images were obtained using a 3.0-T MR scanner (MAGNETOM Prisma; Siemens, Munich, Germany). In order to evaluate signal changes in treated IVDs, the present inventors scored the degree of IVD degeneration, using the Pfirrmann classification (36), which includes 5 grades (1: normal to 5: highly degenerated). Furthermore, MRI index value (the product of the mean signal intensity of the NP and the NP area) was measured using Analyze 14.0 software (AnalyzeDirect, Overland Park, KS, USA), and the brightness of the NP tissues was quantified. Further, in all of the three treatment groups (2, 3, 15, 37, and 38), the relative MRI index, which is the ratio of the MRI index in the intact control group, was evaluated. DHI, which is the ratio of the height of the intervertebral disc to the height of the adjacent vertebral body on the cranial side, was also measured (4 and 39). Relative DHI, which is the percentage of the DHI in the intact control group, was determined.

### Histological Analysis

After MRI imaging, the samples were fixed in 10% formaldehyde, were then desalted with 10% EDTA (pH 7.5), and were then embedded in paraffin. A sagittal 5-micrometer-thick paraffin section was deparaffinized with xylene, were treated with alcohol, were then rinsed with water, and were then stained with H & E and safranin-O. The degree of IVD degeneration was scored from 0 (normal) to 36 (highly degenerated), using the modified Boos' classification (3, 40, and 41).

### IHC

The expression of type II and type I collagens in IVD was determined by IHC. A section was deparaffinized in xylene and was then treated with 0.1% trypsin for 30 minutes for antigen activation. The section was then treated with 3% H₂O₂ in methanol for 10 minutes, followed by protein blocking for 30 minutes using a protein block serum-free solution (DAKO, Agilent, Santa Clara, CA, USA). Goat anti-type I collagen (1:40; Southern Biotech, Birmingham, AL, USA) was used together with an anti-type I collagen antibody, and anti-hCL(II) and purified IgG (1:400; Kyowa Pharma Chemical Co. Ltd., Toyama, Japan) were used as primary antibodies together with an anti-type II collagen antibody. After washing the cells with PBS, EnVision+System-HRP-labeled polymer anti-mouse (DAKO) for type II collagen and Histofine (registered trademark) Simple Stain Max PO(G) (Nichirei Biosciences, Tokyo, Japan) for type I collagen were used as secondary antibodies. Finally, the section was stained with DAB (DAKO) and hematoxylin. The number of type II and type I collagen positive cells was determined in five randomly selected fields of view, and the percentage of the positive cells in all cells was calculated (2, 3, and 15).

### Tumorigenesis Analysis

In order to evaluate the tumorigenesis of transplanted cells and existing cells in transplanted IVDs, (i) invasive proliferation, (ii) nuclear division, (iii) binucleate cells, and (iv) nucleoli were evaluated.

During the 24-week evaluation period, using H & E-stained specimens in the intact control and RECs+gel groups, the total number of positive cells was determined in 15 randomly selected visualization fields (15 visualization fields at 400× magnification; 26.5 visualization fields, a total of 5 mm 2), and the number of the cells per square millimeter was calculated.

### 2. Results

### 2.1. Results of In Vitro 3D Co-Culture of RECs or Commercially Available Human BMSCs and Human NPCs

### Comparison Between RECs and Commercially Available Human BMSCs

RECs and commercially available human BMSCs were confirmed in terms of safety, before and after incorporation thereof into gel. The results are shown in Tables 2 and 3.

The ratio of PI-unstained cells to the total number of cells was defined as cell viability. There was no significant difference between RECs and BMSCs, in terms of the ratio (p=0.6087, Table 2). The coefficient of variation (CV) of a forward scattered light proportional to a cell diameter was calculated, and the uniformity of the cell size was evaluated. The % CV value was significantly lower in the RECs than in the BMSCs (p=0.0366, Table 2). Cell proliferation ability was evaluated by calculating Td. Td was significantly shorter in the RECs than in the BMSCs (p=0.0074, Table 2). The cell surface antigen confirmation test showed that the percentage of CD90-positive cells was significantly higher in the RECs than in the BMSCs (p=0.0004). There was no significant difference in CD45 positive cells between the groups (p=0.0907, Table 2).

The present inventors further compared between the RECs and the commercially available BMSCs after the culture for 1 week (Table 3). There was no significant difference between the RECs and the BMSCs in terms of Td after the 2D and 3D cultures (p=0.3339 and p=0.6996, respectively). However, the percentage of CD90 positive cells was significantly higher in the RECs than in the BMSCs in both of the 2D and 3D cultures (p=0.0039 and p=0.0007, respectively). With regard to the percentage of CD45-positive cells, the percentage thereof was significantly lower in the RECs than in the BMSCs after the 2D culture (p=0.0038). There was no significant difference in terms of the percentage of CD45-positive cells between the RECs and the BMSCs after the 3D culture (p=0.3221). In the 3D culture environment with alginate, which mimics in vitro the cellular environment after transplantation into the intervertebral disc, the expression of cell surface markers characteristic for mesenchymal stem cells was maintained in the RECs, compared with in the BMSCs. These results demonstrate that RECs stably maintain mesenchymal stem cell traits even in an alginate environment.

TABLE 2
Comparison of RECs with commercially available human BMSCs

| | REC | BMSC | P value |
|---|---|---|---|
| Cell viability confirmation test (%) | 94.7 ± 1.1 | 94.0 ± 1.8 | 0.6087 |
| Cell size confirmation test (%) | 34.9 ± 1.0 | 40.5 ± 3.0 | 0.0366 |
| Cell doubling time (hr) | 34.5 ± 5.4 | 51.9 ± 2.6 | 0.0074 |
| Cell surface antigen confirmation | 99.6 ± 0.1 | 98.3 ± 0.2 | 0.0004 |
| test (%) | | | |
| CD90 (above), CD45 (below) | 0.0 ± 0.0 | 0.03 ± 0.02 | 0.0907 |

The data are shown in the form of a mean value ± SD (n = 3).
The P value was determined according to a Student's t-test.

TABLE 3
Comparison of RECs with commercially available
BMSCs after culture for 1 week

| | | REC | BMSC | P value |
|---|---|---|---|---|
| Cell doubling | 2D plate culture | 54.2 ± 14.9 | 44.7 ± 1.5 | 0.3339 |
| time (hr) | 3D culture in | 57.9 ± 14.5 | 54.2 ± 5.0 | 0.6996 |
| | UPAL gel | | | |
| CD90 (%) | 2D plate culture | 99.6 ± 0.1 | 99.0 ± 0.1 | 0.0039 |
| | 3D culture in | 92.8 ± 6.6 | 49.0 4.6 | 0.0007 |
| | UPAL gel | | | |
| CD45 (%) | 2D plate culture | 0.0 ± 0.0 | 0.05 ± 0.02 | 0.0038 |
| | 3D culture in | 0.07 ± 0.06 | 0.03 ± 0.02 | 0.3221 |
| | UPAL gel | | | |

The data are shown in the form of a mean value ± SD (n = 3).
The P value was determined according to a Student's t-test.

### Co-Culture of RECs and Human NP Cells (NPCs)

The present inventors have examined the effect of co-culturing RECs and human NPCs, and the effect of co-culturing different cell types, commercially available human BMSCs and human NPCs, and have then made a comparison between these cells. RECs or BMSCs labeled with 5,6-caboxy fluorescein diacetate succinimidyl ester (CFDA-SE), and non-labeled NPCs were embedded in UPAL gel, and were then subjected to three-dimensional (3D) culture (2, 15, and 27). After the culture for 7 days, the gels were lysed, and residues and dead cells were removed using a cell sorting machine. Subsequently, the cells were classified into CFDA-SE positive cells, RECs, BMSCs, non-labeled cells, and NPCs. After the cell sorting, the following 10 types of cells were obtained.
(a) Control NPCs (not cultured)
(b) Monocultured NPCs
(c) NPCs co-cultured with commercially available BMSCs
(d) NPCs co-cultured with RECs
(e) Control BMSCs (not cultured)
(f) Monocultured BMSCs
(g) Co-cultured BMSCs
(h) Control RECs (not cultured)
(i) Monocultured RECs
(j) Co-cultured RECs

In order to analyze differentiation of BMSCs and RECs, intercellular interaction and ECM production, the expression levels of NPC markers (including HIF-1α, GLUT-1 and brachyury), growth factors (including CDMP-1, TGF-β and IGF-1) and ECM components (including type II collagen and aggrecan) were measured by real-time quantitative reverse transcription polymerase chain reaction (qRT-PCR) (Table 4) (2, 9, 30, and 31).

TABLE 4
Primer and probe mixtures

| Gene | Gene symbol | Assay ID |
|---|---|---|
| HIF-1α | HIF1A | Hs00153153_m1 |
| GLUT-1 | SLC2A1 | Hs00892681_m1 |
| Brachyury | T | Hs00610080_m1 |
| CDMP-1 | GDF5 | Hs00167060_m1 |
| TGF-β1 | TGFB1 | Hs00998133_m1 |
| IGF-1 | IGF1 | Hs01547656_m1 |
| Type II collagen | COL2A1 | Hs00264051_m1 |
| Aggrecan | ACAN | Hs00153936_m1 |
| GAPDH | GAPDH | Hs02786624_g1 |

The expression levels of the three NPC markers, HIF-1α, GLUT-1 and brachyury, were significantly higher in NPCs co-cultured with RECs than in control NPCs, monocultured NPCs, and NPCs co-cultured with commercially available BMSCs. The expression level of GLUT-1 was significantly higher in the NPCs co-cultured with the commercially available BMSCs than in the control NPCs (FIG. 13, D to F). The expression levels of CDMP-1 and IGF-1 were significantly increased in the NPCs co-cultured with RECs, compared with in the control NPCs, the monocultured NPCs, and the NPCs co-cultured with commercially available BMSCs. The expression level of TGF-β was significantly increased in the NPCs co-cultured with RECs, compared with that in the control NPCs and the mono-cultured NPCs. The NPCs co-cultured with commercially available BMSCs showed a significant increase in CDMP-1 and IGF-1 expression, compared with the control NPCs and the monoculture NPCs (FIG. 13, G to I). The expression levels of type II collagen and aggrecan were significantly higher in the NPCs co-cultured with RECs than in the control NPCs and the monocultured NPCs, and also, the expression levels of type II collagen and aggrecan were significantly higher in the NPCs co-cultured with commercially available BMSCs than in the control NPCs and the monocultured NPCs (FIGS. 13, J and K).

The expression levels of all of the three NPC markers in the co-cultured RECs were significantly higher than those in the control and monocultured RECs (FIGS. 13, D to F). The expression levels of CDMP-1, TGF-β and IGF-1 were also significantly increased in the co-cultured RECs, compared with the control and monocultured RECs (FIG. 13, G to I). Similarly, the expression levels of type II collagen and aggrecan were significantly higher in the co-cultured RECs than in the control and monocultured RECs (FIGS. 13, J and K).

### 2.2. Results of Elasticity Analysis of Gels Using Unconfined Compression Test

### Effects of RECs Embedded in UPAL Gel

In order to evaluate and compare the mechanical properties of REC-UPAL and UPAL gel, an unconfined compression test was performed (3, 15, 32, and 33). Two types of cylindrical gel samples (a diameter of 4.5 mm and a thickness of 2 mm) were prepared, and the ratio of axial compressive stress to strain (FIG. 14, A to C) was calculated. At a compression level of 10% to 20%, Young's modulus was 18.0±3.5 kPa and 18.8±2.1 kPa in UPAL and REC-UPAL gel, respectively, and thus, there was no significant difference in terms of mechanical properties between the two groups (p=0.6942). (FIGS. 14, D and E). Furthermore, these two values were equivalent to those of normal human NP tissues (3 and 32).

### 2.3. Results of In Vivo Test Using Degenerated IVD Sheep Models

### Enhancement of IVD Regeneration by Combined Use of RECs and UPAL Gel

The regeneration ability of IVDs was evaluated using sheep models after transplantation of gel or RECs+gel (FIG. 15). Sheep have been widely used in studies regarding IVDs after discectomy, including biomaterials (3, 34, and 35). Fifty-six IVDs derived from 14 sheep were divided into the following groups.

Intact control group (n=6 IVDs)

Discectomy group (n=6 IVDs)

Gel group (n=8 IVDs)

RECs+gel group (n=8 IVDs)

In order to produce IVD degeneration models, 20 mg of NP tissues were removed during the first surgery (FIGS. 15, A and B), and based on the results of preliminary experiments, the amount to be removed was set to be 20 mg (FIG. 19). At 4 weeks after the first surgery (after observation of degenerative changes), NP tissues were removed again (FIG. 15C). Subsequently, UPAL or a RECs+UPAL solution was transplanted into the IVD defect, and after the second discectomy, 102 mM CaCl₂ was exposed for gelling (FIG. 15D). The transplanted lumbar vertebrae were collected for various analyses at 4 weeks and 24 weeks after the transplantation.

Degenerative changes in the treated IVDs were evaluated using T2-weighted, central sagittal cross-sectional images obtained via MRI (FIG. 16A). For the morphological analysis of IVDs, the Pfirrmann score and the MRI index were evaluated to assess signal changes in the embedded IVDs (2, 3, 15, and 36 to 38) and intervertebral disc height index (DHI). Based on the Pfirrmann score, the degeneration score of the gel group was significantly lower than that of the discectomy group at 24 weeks, and the degeneration score of the RECs+gel group was significantly lower than that of the discectomy group and the gel group at both 4 weeks and 24 weeks (FIG. 16B).

The MRI index was significantly higher in the RECs+gel group than in the discectomy group at 4 weeks. At 24 weeks, the MRI index of the gel group was significantly higher than that of the discectomy group, and the MRI index of the RECs+gel group was significantly higher than that of the discectomy group and the gel group (FIG. 16C).

Furthermore, the height of the intervertebral disc of the treated IVDs was measured using MRI images. The ratio of the height of an intervertebral disc to the height of the upper adjacent vertebral body was measured, namely, DHI was measured at the anterior portion and posterior portion of the IVD. Subsequently, the relative DHI, namely, the ratio of the DHI values of the three treatment groups to the DHI value of the intact control group, was determined (FIG. 20) (4 and 39). The DHI values of the three treatment groups were significantly lower than the DHI value of the intact control group at both 4 weeks and 24 weeks. At 4 weeks after the transplantation, there was no significant difference among the three groups, but at 24 weeks after the transplantation, the DHI of the gel group was significantly higher than that of the discectomy group, and the DHI value of the RECs+gel group was significantly higher than that of the discectomy group and the gel group (FIG. 16D).

Histological analysis was performed using hematoxylin & eosin (H & E) and safranin-O staining. For both 4 weeks and 24 weeks, in the intact control group, IVD showed a spindle shape, no fibrotic changes were found in NP tissues, and annulus fibrosis (AF) had a concentric structure and was uniformly stained with safranin-O. In the discectomy group, significant scar tissues, fibrotic changes, tissue loss, and disruption and/or destruction of the end plates were found (FIGS. 17, A and B), and the histological degeneration grade determined based on the modified Boos' classification (3, 40, and 41) was significantly lower in the gel group than in the discectomy group for both 4 weeks and 24 weeks, and the histological degeneration grade was significantly lower in the RECs+gel group than in the discectomy group and the gel group for both 4 weeks and 24 weeks (FIG. 17C).

The expression levels of type II and type I collagens in treated IVDs were evaluated according to immunohistochemistry (IHC) (FIGS. 18, A and B). The percentage of type II and type I collagen positive cells in total number of cells in five randomly selected visualized fields of view was evaluated in NP tissues (2, 3, and 15). Type II collagen is an essential ECM component in NP tissues, and is replaced by type I collagen as degeneration progresses. In the analysis of type II collagen, NP tissues were stained uniformly in the intact control group, but the staining level was slightly decreased in the RECs+gel group. Scattering of non-stained areas was observed in the gel group, and broad non-stained areas were observed in the discectomy group. The percentages of the type II collagen positive cells were significantly higher in the RECs+gel group than in the gel group and the discectomy group at both 4 weeks and 24 weeks. At 24 weeks, the percentage of the type II collagen positive cells was significantly higher in the gel group than in the discectomy group (FIG. 18C). In contrast, the percentage of type I collagen positive cells was significantly lower in the RECs+gel group than in the discectomy group during the 4-week evaluation period, and was significantly lower in the RECs+gel group than in the discectomy group and the gel group during the 24-week evaluation period. Moreover, the percentage of these cells was significantly lower in the gel group than in the discectomy group at 24 weeks (FIG. 18D).

### Influence of RECs+Gel Injection on IVD

During the 24-week evaluation period, tumorigenesis was analyzed in histological specimens from the intact control group and the RECs+gel group. In all specimens of both groups, invasive proliferation was evaluated as well as nuclear fission, and the number of binucleate cells and the number of nucleoli per square millimeter were measured. No invasive proliferation, no nuclear fission image, and no nucleoli were observed in both groups. The number of binucleate cells in the intact control group and in the RECs+gel group were 0.03±0.07 and 0.23±0.25, respectively. That is to say, the values were <1/mm 2 and no significant difference was observed between the groups (p=0.3311) (Table 5).

TABLE 5
Number of binucleate cells per square millimeter

| Intact control | RECs + gel | P value |
|---|---|---|
| 0.03 ± 0.07 | 0.23 ± 0.25 | 0.3311 |

The data are shown in the form of a mean value ± standard deviation, and after a Welch test, the P value was obtained according to a U test of Mann-Whitney.

### 3. Consideration

In the present example, with regard to cell proliferation ability, cell size uniformity, and the expression of cell surface antigens, RECs were compared with commercially available human BMSCs, and as a result, the excellent characteristics of the RECs were demonstrated. Furthermore, in the present example, it was demonstrated that the expression levels of NPC markers, growth factors, and ECM components were significantly increased in the 3D co-culture of human NPCs and RECs, compared with the expression levels thereof observed in the 3D co-cultures of commercially available BMSC and NPCs. The efficacy of the combined use of RECs and UPAL gel was observed at the site of IVD degeneration in a sheep lumbar spine model. UPAL gel alone suppressed IVD degeneration, compared with the discectomy group, but the combined use of RECs and the gel more effectively enhanced IVD regeneration.

In the present example, it was suggested that the co-culture of RECs with NPCs leads to the differentiation of the RECs into NPCs, thereby improving the production of ECM components in both cell types. Similar results were observed in the results obtained using rabbits (Example 1). That is, in Example 1, it was elucidated that the expression of the NPC markers was increased after transplantation of BMSCs embedded in UPAL gel, and that the production of ECMs was increased in the BMSCs+UPAL gel group, compared with the discectomy group (2).

Based on the above-described results and the previous study results (2, 31, 47, 48, and 50 to 52), the following possibilities were suggested as mechanisms that serve as bases of IVD regeneration by the transplantation of RECs embedded in UPAL gel.
1) RECs can be embedded in a gel and can be transplanted into an IVD defect.
2) RECs produce growth factors and ECM components, thereby activating existing NPCs (paracrine mechanism).
3) NPCs produce more ECMs and growth factors.
4) As a result, RECs directly differentiate into NPCs.
5) Finally, RECs and NPCs have a positive intercellular feedback loop that leads to IVD regeneration.

Biomaterial/hydrogel used in IVD repairing need to be biologically and mechanically suitable.

In the present example, since the biomechanical and geometric arrangement of the lumbar IVD of a sheep model is equivalent to those of a human model (3, 4, and 34), a sheep lumbar spine model was selected for transplantation of a candidate hydrogel into a preclinical animal model. To date, the present inventors have demonstrated that UPAL gel embedded in a post-discectomy sheep lumbar IVD exhibits appropriate biomechanical properties, does not provide material protrusion, and does not require the suturing of the AF after discectomy (3). In the present example, the unconfined compression test revealed that there is no significant difference in Young's modulus between the UPAL group and the RECs-UPAL group, demonstrating that the RECs embedded in the UPAL gel did not change the mechanical properties of the gel. Because of definitive ability to provide rapid healing, the combination of UPAL gel and RECs offers a clinical advantage in terms of preventing cell leakage without suturing the AF.

MRI and histological evaluation in animals transplanted (and not transplanted) with UPAL gel showed that IVD degeneration was suppressed after transplantation of UPAL gel alone. Furthermore, the combination of RECs and UPAL gel prevented degeneration more effectively than UPAL gel alone. Further, there was no significant difference in terms of the number of binucleate cells between the non-treated group and the RECs+gel group, suggesting that the combination of RECs and gel does not induce tumorigenesis. Further, the IHC results suggest that the combined use of RECs and gel enhanced the synthesis of ECMs essential for normal IVD function, and suppressed production of type I collagen, so that the progression of IVD degeneration was inhibited. These results are consistent with the results obtained using the rabbit models of IVD degeneration (Example 1), and demonstrate that BMSCs embedded in UPAL gel suppressed degeneration more effectively (2). Example 1 also showed that the histological degeneration score after transplantation of BMSCs+UPAL gel was low, compared with animals that did not undergo discectomy, in which a degenerated IVD was produced via AF needle puncture. This suggests that BMSC transplantation resulted in IVD regeneration (2). To sum up, the findings of the present inventors indicate that transplantation of RECs embedded in UPAL gel enhances IVD regeneration in vivo (2).

### REFERENCE DOCUMENTS IN EXAMPLE 4

1. J. N. Katz, Lumbar disc disorders and low-back pain: Socioeconomic factors and consequences. J. Bone Joint Surg. Am. 88, 21-24 (2006).
2. D. Ukeba, H. Sudo, T. Tsujimoto, K. Ura, K. Yamada, N. Iwasaki, Bone marrow mesenchymal stem cells combined with ultra-purified alginate gel as a regenerative therapeutic strategy after discectomy for degenerated intervertebral discs. EBioMedicine 53, 102698 (2020).
3. T. Tsujimoto, H. Sudo, M. Todoh, K. Yamada, K. Iwasaki, T. Ohnishi, N. Hirohama, T. Nonoyama, D. Ukeba, K. Ura, Y. M. Ito, N. Iwasaki, An acellular bioresorbable ultra-purified alginate gel promotes intervertebral disc repair: A preclinical proof-of-concept study. EBioMedicine 37, 521-534 (2018).
4. S. R. Sloan Jr, C. Wipplinger, S. Kirnaz, R. Navarro-Ramirez, F. Schmidt, D. McCloskey, T. Pannellini, A. Schiavinato, R. Hartl, I. J. Bonassar, Combined nucleus pulposus augmentation and annulus fibrosus repair prevents acute intervertebral disc degeneration after discectomy. Sci. Transl. Med. 12, eaay2380 (2020).
5. P. Heindel, A. Tuchman, P. C. Hsieh, M. H. Pham, A. D'Oro, N. N. Patel, A. M. Jakoi, R. Hah, J. C. Liu, Z. Buser, J. C. Wang, Reoperation rates after single-level lumbar discectomy. Spine 42, E496-E501 (2017).
6. J. Sherman, J. Cauthen, D. Schoenberg, M. Burns, N. L. Reaven, S. L. Griffith, Economic impact of improving outcomes of lumbar discectomy. Spine J. 10, 108-116 (2010).
7. P. Kambin, L. F. Cohen, M. Brooks, J. L. Schaffer, Development of degenerative spondylosis of the lumbar spine after partial discectomy. Comparison of laminotomy, discectomy, and posterolateral discectomy. Spine 20, 599-607 (1995).
8. D. Oehme, P. Ghosh, S. Shimmon, J. Wu, C. McDonald, J. M. Troupis, T. Goldschlager, J. V. Rosenfeld, G. Jenkin, Mesenchymal progenitor cells combined with pentosan polysulfate mediating disc regeneration at the time of microdiscectomy: a preliminary study in an ovine model. J. Neurosurg. Spine 20, 657-669 (2014).
9. S. M. Naqvi, C. T. Buckley, Differential response of encapsulated nucleus pulposus and bone marrow stem cells in isolation and coculture in alginate and chitosan hydrogels. Tissue Eng. Part A 21, 288-99 (2015).
10. D. C. Noriega, F. Ardura, R. Hernandez-Ramajo, M. A. Martin-Ferrero, I. Sanchez-Lite, B. Toribio, M. Alberca, V. Garcia, J. M. Moraleda, A. Sanchez, J. Garcia-Sancho, Intervertebral disc repair by allogeneic mesenchymal bone marrow cells: A randomized controlled trial. Transplantation 101, 1945-1951 (2017).
11. R. D. Bowles, H. H. Gebhard, R. Hartl, L. J. Bonassar, Tissue-engineered intervertebral discs produce new matrix, maintain disc height, and restore biomechanical function to the rodent spine. Proc. Natl. Acad. Sci. U.S.A. 108, 13106-13111 (2011).
12. P. A. Revell, E. Damien, L. Di Silvio, N. Gaurav, C. Longinotti, L. Ambrosio, Tissue engineered intervertebral disc repair in the pig using injectable polymers. J. Mater. Sci. Mater. Med. 18, 303-308 (2007).
13. I. Hussain, S. R. Sloan, C. Wipplinger, R. Navarro-Ramirez, M. Zubkov, E. Kim, S. Kirnaz, L. J. Bonassar, R. Hartl, Mesenchymal stem cell-seeded high-density collagen gel for annular repair: 6-week results from in vivo sheep models. Neurosurgery 85, E350-E359 (2018).
14. B. Pennicooke, I. Hussain, C. Berlin, S. R. Sloan, B. Borde, Y. Moriguchi, G. Lang, R. Navarro-Ramirez, J. Cheetham, L. J. Bonassar, R. Hartl, Annulus fibrosus repair using high-density collagen gel: An in vivo ovine model. Spine 43, E208-E215 (2018).
15. D. Ukeba, K. Yamada, T. Tsujimoto, K. Ura, T. Nonoyama, N. Iwasaki, H. Sudo, Bone marrow aspirate concentrate combined with in situ forming bioresorbable gel enhances intervertebral disc regeneration in rabbits. J. Bone Joint Surg. Am. 103, e31 (2021).
16. K. Ura, K. Yamada, T. Tsujimoto, D. Ukeba, N. Iwasaki, H. Sudo, Ultra-purified alginate gel implantation decreases inflammatory cytokine levels, prevents intervertebral disc degeneration, and reduces acute pain after discectomy. Sci. Rep. 11, 638 (2021).
17. A. J. Friedenstein, U. F. Deriglasova, N. N. Kulagina, A. F. Panasuk, S. F. Rudakowa, E. A. Luria, I. A. Ruadkow, Precursors for fibroblasts in different populations of hematopoietic cells as detected by the in vitro colony assay method. Exp. Hematol. 2, 83-92 (1974).
18. Y. Mabuchi, S. Morikawa, S. Harada, K. Niibe, S. Suzuki, F. Renault-Mihara, D. D. Houlihan, C. Akazawa, H. Okano, Y. Matsuzaki, LNGFR(+)THY-1(+)VCAM-1(hi+) cells reveal functionally distinct subpopulations in mesenchymal stem cells. Stem Cell Rep. 1, 152-165 (2013).
19. M. F. Pittenger, A. M. Mackay, S. C. Beck, R. K. Jaiswal, R. Douglas, J. D. Mosca, M. A. Moorman, D. W. Simonetti, S. Craig, D. R. Marshak, Multilineage potential of adult human mesenchymal stem cells. Science 284, 14-147 (1999).
20. J. Kim, J. W. Kang, J. H. Park, Y. Choi, K. S. Choi, K. D. Park, D. H. Baek, S. K. Seong, H. K. Min, H. S. Kim, Biological characterization of long-term cultured human mesenchymal stem cells. Arch. Pharm. Res. 32, 117-126 (2009).
21. W. J. C. Rombouts, R. E. Ploemacher, Primary murine MSC show highly efficient homing to the bone marrow but lose homing ability following culture. Leukemia 17, 160-170 (2003).
22. C. Brown, C. McKee, S. Bakshi, K. Walker, E. Hakman, S. Halassy, D. Svinarich, R. Dodds, C. K. Govind, G. R. Chaudhry, Mesenchymal stem cells: Cell therapy and regeneration potential. J. Tissue Eng. Regen. Med. 13, 1738-1755 (2019).
23. T. Squillaro, G. Peluso, U. Galderisi, Clinical trials with mesenchymal stem cells: An update. Cell Transplant. 25, 829-848 (2016).
24. S. Morikawa, Y. Mabuchi, Y. Kubota, Y. Nagai, K. Niibe, E. Hiratsu, S. Suzuki, C. M.-Hara, N. Nagoshi, T. Sunabori, S. Shimmura, A. Miyawaki, T. Nakagawa, T. Suda, H. Okano, Y. Matsuzaki, Prospective identification, isolation, and systemic transplantation of multipotent mesenchymal stem cells in murine bone marrow. J. Exp. Med. 11, 2483-2496 (2009).
25. D. D. Houlihan, Y. Mabuchi, S. Morikawa, K. Niibe, D. Araki, S. Suzuki, H. Okano, Y. Matsuzaki, Isolation of mouse mesenchymal stem cells on the basis of expression of Sca-1 and PDGFR-α. Nat. Protoc. 7, 2103-2111 (2012).
26. Y. Mabuchi, Y. Matsuzaki, Prospective isolation of resident adult human mesenchymal stem cell population from multiple organs. Int. J. Hematol. 103, 138-144 (2016).
27. S. Harada, Y. Mabuchi, J. Kohyama, D. Shimojo, S. Suzuki, Y. Kawamura, D. Araki, T. Suyama, M. Kajikawa, C. Akazawa, H. Okano, Y. Matsuzaki, FZDS regulates cellular senescence in human mesenchymal stem/stromal cells. Stem Cells 39, 318-330 (2021).
28. K. Yamada, K. Maeda, Y. M. Ito, F. Inage, T. Isoe, N. Yokota, O. Sugita, N. Sato, K. K. Tha, N. Iwasaki, H. Sudo, Exploratory clinical trial on the safety and capability of dMD-001 in lumbar disc herniation: Study protocol for a first-in-human pilot study. Contemp Clin Trials Commun. 23, 100805 (2021).
29. M. Sato, K. Uchida, H. Nakajima, T. Miyazaki, A. R. Guerrero, S. Watanabe, S. Roberts, H. Baba, Direct transplantation of mesenchymal stem cells into the knee joints of Hartley strain guinea pigs with spontaneous osteoarthritis. Arthritis Res. Ther. 14, R31 (2012).
30. M. V. Risbud, Z. R. Schoepflin, F. Mwale, R. A. Kandel, S. Grad, J. C. Iatridis, D. Sakai, J. A. Hoyland, Defining the phenotype of young healthy nucleus pulposus cells: recommendations of the Spine Research Interest Group at the 2014 annual ORS meeting. J Orthop. Res. 33, 283-293 (2015).
31. S. Strassburg, S. M. Richardson, A. J. Freemont, J. A. Hoyland. Co-culture induces mesenchymal stem cell differentiation and modulation of the degenerate human nucleus pulposus cell phenotype. Regen. Med. 5, 701-711 (2010).
32. J. C. Iatridis, M. Weidenbaum, L. A. Setton, V. C. Mow, Is the nucleus pulposus a solid or a fluid? Mechanical behaviors of the nucleus pulposus of the human intervertebral disc. Spine 21, 1174-1184 (1996).
33. Y. Zeng, C. Chen, W. Liu, Q. Fu, Z. Han, Y. Li, S. Feng, X. Li, C. Qi, J. Wu, D. Wang, C. Corbett, B. P. Chan, D. Ruan, Y. Du, Injectable microcryogels reinforced alginate encapsulation of mesenchymal stromal cells for leak-proof delivery and alleviation of canine disc degeneration. Biomaterials 59, 53-65 (2015).
34. S. Reitmaier, H. Schmidt, R. Ihler, T. Kocak, N. Graf, A. Ignatius, H.-J. Wilke, Preliminary investigations on intradiscal pressures during daily activities: an in vivo study using the merino sheep. PLoS One 8, e69610 (2013).
35. A. A. Hegewald, F. Medved, D. Feng, C. Tsagogiorgas, A. Beierfuβ, G. A. Schindler, M. Trunk, C. Kaps, D. S. Mern, C. Thome, Enhancing tissue repair in annulus fibrosus defects of the intervertebral disc: Analysis of a bio-integrative annulus implant in an in-vivo ovine model. J. Tissue Eng. Regen. Med. 9, 405-414 (2015).
36. C. W. Pfirrmann, A. Metzdorf, M. Zanetti, J. Hodler, N. Boos, Magnetic resonance classification of lumbar intervertebral disc degeneration. Spine 26, 1873-1878 (2001).
37. H. Sudo, A. Minami, Caspase 3 as a therapeutic target for regulation of intervertebral disc degeneration in rabbits. Arthritis Rheum. 63, 1648-1657 (2011).
38. K. Ura, H. Sudo H, K. Iwasaki, T. Tsujimoto T, D. Ukeba, N. Iwasaki N, Effects of intradiscal injection of local anesthetics on intervertebral disc degeneration in rabbit degenerated intervertebral disc. J. Orthop. Res. 37, 1963-1971 (2019).
39. D. S. Lu, Y. Shono, I. Oda, K. Abumi, K. Kaneda, Effects of chondroitinase ABC and chymopapain on spinal motion segment biomechanics. An in vivo biomechanical, radiologic, and histologic canine study. Spine 22, 1828-1834 (1997).
40. N. Boos, S. Weissbach, H. Rohrbach, C. Weiler, K. F. Spratt, A. G. Nerlich, Classification of age-related changes in lumbar intervertebral discs: 2002 Volvo Award in basic science. Spine 27, 2631-2644 (2002).
41. S. Reitmaier, L. Kreja, K. Gruchenberg, B. Kanter, J. Silva-Correia, J. M. Oliveira, R. L. Reis, V. Perugini, M. Santin, A. Ignatius, H. J. Wilke, In vivo biofunctional evaluation of hydrogels for disc regeneration. Eur. Spine J. 23, 19-26 (2014).
42. H. Wang, Y. Zhou, B. Huang, L.-T. Liu, M.-H. Liu, J. Wang, C.-Q. Li, Z.-F. Zhang, T.-W. Chu, C.-J. Xiong, Utilization of stem cells in alginate for nucleus pulposus tissue engineering. Tissue Eng. Part A 20, 908-920 (2014).
43. F. Wang, L.-P. Nan, S.-F. Zhou, Y. Liu, Z.-Y. Wang, J.-C. Wang, X.-M. Feng, L. Zhang, Injectable hydrogel combined with nucleus pulposus-derived mesenchymal stem cells for the treatment of degenerative intervertebral disc in rats. Stem Cells Int. 2019, U.S. Pat. No. 8,496,025 (2019).
44. G. W. Omlor, S. Lorenz, A. G. Nerlich, T. Guehring, W. Richter, Disc cell therapy with bone marrow-derived autologous mesenchymal stromal cells in a large porcine disc degeneration model. Eur. Spine J. 27, 2639-2649 (2018).
45. M. Dominici, K. L. Blanc, I. Mueller, I. Slaper-Cortenbach, F. Marini, D. Krause, R. Deans, A. Keating, D. Prockop, E. Horwitz, Minimal criteria for defining multipotent mesenchymal stromal cells. The International Society for Cellular Therapy position statement. Cytotherapy 8, 315-317 (2006).
46. C. M. Raynaud, M. Maleki, R. Lis, B. Ahmed, I. Al-Azwani, J. Malek, F. F. Safadi, A. Rafii, Comprehensive characterization of mesenchymal stem cells from human placenta and fetal membrane and their response to osteoactivin stimulation. Stem Cells Int. 2012, 658356 (2012).
47. Y. Yamamoto, J. Mochida, D. Sakai, T. Nakai, K. Nishimura, H. Kawada, T. Hotta, Upregulation of the viability of nucleus pulposus cells by bone marrow-derived stromal cells: significance of direct cell-to-cell contact in coculture system. Spine 29, 1508-1514 (2004).
48. S.-H. Yang, C.-C. Wu, T. T.-F. Shih, Y.-H. Sun, F.-H. Lin, In vitro study on interaction between human nucleus pulposus cells and mesenchymal stem cells through paracrine stimulation. Spine 33, 1951-1957 (2008).
49. A. Ouyang, A. E. Cerchiari, X. Tang, E. Liebenberg, T. Alliston, Z. J. Gartner, J. C. Lotz, Effects of cell type and configuration on anabolic and catabolic activity in 3D co-culture of mesenchymal stem cells and nucleus pulposus cells. J. Orthop. Res. 35, 61-73 (2017).
50. M. V. Risbud, T. J. Albert, A. Guttapalli, E. J. Vresilovic, A. S. Hillibrand, A. R. Vaccaro, I. M. Shapiro, Differentiation of mesenchymal stem cells towards a nucleus pulposus-like phenotype in vitro: implications for cell-based transplantation therapy. Spine 29, 2627-2632 (2004).
51. S. M. Richardson, R. V. Walker, S. Parker, N. P. Rhodes, J. A. Hunt, A. J. Freemont, J. A. Hoyland, Intervertebral disc cell-mediated mesenchymal stem cell differentiation. Stem Cells 24, 707-716 (2006).
52. D. Sakai, J. Mochida, T. Iwashina, T. Watanabe, T. Nakai, K. Ando, T. Hotta, Differentiation of mesenchymal stem cells transplanted to a rabbit degenerative disc model: Potential and limitations for stem cell therapy in disc regeneration. Spine 30, 2379-2387 (2005).
53. T. J. DiStefano, J. O. Shmukler, G. Danias, J. C. latridis, The functional role of interface tissue engineering in annulus fibrosus repair: bridging mechanisms of hydrogel integrationwith regenerative outcomes. ACS Biomater. Sci. Eng. 6, 6556-6586 (2020).
54. T. C. Schmitz, E. Salzer, J. F. Crispim, G. T. Fabra, C. LeVisage, A. Pandit, M. Tryfonidou, C. L. Maitre, K. Ito, Characterization of biomaterials intended for use in the nucleus pulposus of degenerated intervertebral discs. Acta Biomater. 114, 1-15 (2020). 573

### Example 5

Inflammation and pain suppressing effect of sodium alginate and mesenchymal stem cells in rat intervertebral disc nucleus pulposus defect models

### 1. Production of Rat Intervertebral Disc Puncture Degeneration Models

Rat intervertebral disc puncture degeneration models (Mohd Isa et al. Sci Adv 2018) are used in an experiment regarding administration of a test substance (IHC analysis, histological analysis, pain-related behavior analysis). A total of sixty 12-week-old female SD rats (260 to 300 g) are randomly assigned to: a group in which only a skin incision is performed (sham group); a group in which only intervertebral disc puncture is performed (punch group); a group in which UPAL is embedded after intervertebral disc puncture (UPAL group); and a group in which UPAL and RECs are embedded after intervertebral disc puncture (RECs group) (n=3, in each time point and each group). After introduction of anesthesia by 5% isoflurane inhalation, anesthesia is maintained by intraperitoneal injection of a mixture of ketamine and medetomidine (ketamine:medetomidine=75 mg/kg:0.5 mg/kg). After confirming the depth of anesthesia by a tail pinch test with forceps, the dorsal skin of Co4/5-5/6 is incised. The connective tissues are removed to expose Co4/5-5/6, and the intervertebral disc of Co4/5-5/6 is injured using a 19 G needle (a diameter of 1 mm, and a depth of 2 mm).

In the gel group and the RECs groups, dry fibrous UPAL (400 to 600 mPa/s, Mochida Pharma Co., Ltd., Tokyo, Japan) is dissolved in a normal saline (0.9%, Otsuka) to prepare a 2% (w/v) solution, and 4 μl of the UPAL solution is injected into the center of the intervertebral disc, using a microsyringe (Hamilton) fitted with a 26 G needle, via a different route from the puncture site. In the RECs group, the concentration is set to be 1×10⁶/ml. The surgical site is washed with a normal saline, and the fascia, connective tissues, and skin are sutured and closed.

### 2. Immunohistological Evaluation

Immunohistological evaluation of rat intervertebral discs is performed, and at 1, 4, 7 and 28 days after the surgery, the cells that are positive to TNF-α, IL-6 and TrkA are detected. Rats (n=3, in each time point and each group) are deeply anesthetized by isoflurane inhalation and are euthanized by cervical dislocation.

The entire tail (Co 4/5-Co 5/6) is surgically excised, and soft tissues are then removed under aseptic conditions to collect only the caudal vertebrae and intervertebral discs. The collected intervertebral discs are fixed with 4% (w/v) paraformaldehyde (for 48 hours at room temperature) and are then embedded in paraffin. The specimen is transected at the center of the intervertebral disc, so as to obtain a mid-coronal transverse section (5 μm thick). The section is deparaffinized with xylene and is then cultured in proteinase K (Dako, Agilent Technologies, Santa Clara, CA, USA) (at 37° C. for 15 minutes). Subsequently, the cells are blocked with 1% hydrogen peroxide in methanol (w/v) (at 37° C. for 30 minutes), and are then cultured in 2% (w/v) bovine serum albumin (at room temperature for 30 minutes). Thereafter, the resulting cells are cultured with a primary antibody at 4° C. overnight. An anti-TNF-α mouse monoclonal antibody (ab220210, Abcam), an anti-IL-6 mouse monoclonal antibody (ab9324, Abcam), and an anti-TrkA rabbit monoclonal antibody (ab 86474, Abcam) are used.

Regarding coloration, Histofine (registered trademark) Fast Red II (Nichirei Bioscience) is used in TNF-α analysis; HistoGreen Substrate kit for Peroxidase (Cosmo Bio Co. Cosmo Bio Co., Ltd., Tokyo, Japan) is used in IL-6 analysis; and Histofine (registered trademark) DAB (Nichirei Bioscience) is used in TrkA analysis. For the purpose of improving visibility, cell nuclei are subjected to contrast staining. In TNF-α or TrkA staining, hematoxylin is used, and in IL-6 staining, Fast Red is used, respectively. Using a light microscope (Olympus, Tokyo, Japan), the number of cells positive to TNF-α, IL-6 or TrkA is individually counted in five randomly selected fields of view, and the number of positive nucleus pulposus or annulus fibrosis cells in each staining is calculated as a percentage to the total number of nucleus pulposus or annulus fibrosis cells in the field of view. All evaluations are performed by two independent blinded observers. Each observer performs three evaluations on one specimen, calculates a mean value for each specimen, and makes a comparison among individual groups.

### 3. Histological Evaluation

Degeneration of intervertebral disc tissues at 28 days after the surgery is evaluated. Rats (n=3) are euthanized by the same method as that for the immunohistological evaluation, and intervertebral discs are then collected. The collected intervertebral discs are fixed with 4% (w/v) paraformaldehyde for 48 hours, and are then decalcified with a Kristensen demineralizer for 2 weeks. Thereafter, the resulting intervertebral discs are washed with tap water for 24 hours, and are then embedded in paraffin (Mohd Isa et al. Sci Adv 2018). In order to evaluate the histological score (Rutges et al. Osteoarth Carti 2013) using the mid-sagittal sectioned specimens (5 lam thick) of the rat intervertebral discs, the specimens are stained with hematoxylin & eosin, safranin O, or Alcian blue (AB). The AB staining is not directly associated with the score, but the AB staining is suitable for evaluation of extracellular matrixes. Thus, the AB staining is performed for auxiliary purpose. All evaluations are performed by two independent blinded observers. Each observer performs three evaluations on one specimen, calculates a mean value for each specimen, and makes a comparison among individual groups.

### 4. Pain-Related Behavioral Analysis

A total of 24 rats (n=6 per group), namely, 12 rats (n=3 per group) that are allowed to survive until 28 days after the surgery, and 12 rats (n=3 per group) used in the histological analysis, are used in the analysis of pain-related behaviors. All rats are subjected to a Hargreaves test, a von Frey test, and a tail flick test. Twenty-four hours before each test and immediately before each test, the rats are individually placed in a test environment for 20 minutes so as to acclimate the rats to the environment. All tests are performed by a single blinded examiner. The measurement is performed multiple times for each rat, and a mean value is then calculated. The obtained results are compared among the groups.

### 4.1 Hargreaves Test

The Hargreaves test is performed at two days (Day-2) before the surgery, and at 2, 7, 14, and 27 days after the surgery, using a Hargreaves test device (Ugo Basile Biological Instruments, Gemonio, Italy) (Mohd Isa et al. Sci Adv 2018). Rats are placed in individual small chambers (with air holes above) on a glass plate (Ugo Basile Biological Instruments), in which all sides and above are enclosed. An infrared beam is applied as thermal stimulation to the ventral side of a skin incision portion. A latency until the rats show escape behavior to the thermal stimulation is recorded. The intensity of the beam is set to be 50% of the maximum output. For the purpose of preventing tissue damage, the cut-off time is set to be 20 seconds. Four measurements are performed at each time point on a single rat, with a rest of at least 1 minute between individual measurements.

### 4.2 Von Frey Test

The Von Frey test is performed at two days (Day-2) before the surgery, and at 2, 7, 14, and 27 days after the surgery, using a dynamic plantar aesthesiometer (Ugo Basile Biological Instruments). The same small chambers as those used in the Hargreaves test are established on a wire mesh, and rats are then placed in the chambers. A filament with a diameter of 0.5 mm is placed on the ventral side of a skin incision portion, and a linearly increasing force is applied for 10 seconds starting from 0 g up to 5 g, and thereafter, a constant force of 5 g is applied until 30 seconds after the initiation of the test. A latency until the rats show some escape behavior is recorded. Five measurements are performed at each time point on a single rat, with at least a 10-second rest between individual measurements.

### 4.3 Tail Flick Test

The tail flick test is performed using a heat flux radiometer (manufactured by Ugo Basile Biological Instruments). In order to avoid tissue damage due to excessive thermal stimulation caused by implementation of the test according to the same schedule as the Hargreaves test, the tail flick test is performed at one day before the surgery (Day-1) and at days 3, 8, 15, and 28 after the surgery (Mohd Isa et al. Sci Adv 2018). After each rat is wrapped with a towel and is allowed to settle for 10 minutes, only the tail is placed on the apparatus while the body remains covered with the towel. An infrared beam is applied to the ventral side 5 cm proximal to the distal end of the tail. A latency until a tail shaking reaction to the thermal stimulation is initiated is recorded. The cutoff time is set to be 20 seconds to prevent tissue damage. Four measurements are performed at each time point on a single rat, with at least a 15-second rest between individual measurements.

### 5. Statistical Analysis

All data are shown in the form of a mean value±standard error (SE). One-way ANOVA is used for multiple group comparison. For two-group comparison, A Student-t test without correspondence is carried out. All ANOVA results are further evaluated using a Tukey-Kramer post-hoc test or a Kruskal-Wallis test. A difference is considered to be statistically significant at a significance level of 5% (P<0.05).

### 6. Results

In a group involving the transplantation of UPAL and RECs (RECs group), a pain-suppressing effect can be confirmed.

1. A composition for regeneration of an intervertebral disc, comprising a monovalent metal salt of alginic acid and mesenchymal stem cells.

2. The composition according to claim 1, wherein regeneration of the nucleus pulposus of an intervertebral disc is promoted via activation of nucleus pulposus cells by mesenchymal stem cells and/or differentiation of mesenchymal stem cells into nucleus pulposus cells.

3. The composition according to claim 1 or 2, wherein the mesenchymal stem cells are human bone marrow-derived high-purity mesenchymal stem cells.

4. The composition according to claim 3, wherein the human bone marrow-derived high-purity mesenchymal stem cells are a cell population of LNGFR (CD271)-positive, or LNGFR (CD271) and Thy-1 (CD90) double-positive, rapidly proliferating mesenchymal stem cell clones, said cell population satisfying at least one of the following characteristics (a) and (b):
(a) the coefficient of variation of forward scattered light in flow cytometry is 40% or less; and
(b) the average cell size is 20 nm or less.

5. The composition according to claim 3 or 4, wherein the human bone marrow-derived high-purity mesenchymal stem cells are a cell population of rapidly proliferating mesenchymal stem cell clones that are separated using, as an indicator, the feature that the cells are LNGFR (CD271)-positive, or LNGFR (CD271) and Thy-1 (CD90) double-positive cells, said cell population satisfying at least one of the following characteristics (a) and (b):
(a) the coefficient of variation of forward scattered light in flow cytometry is 40% or less; and
(b) the average cell size is 20 nm or less.

6. The composition according to any one of claims 1 to 5, which is used such that the composition is applied to the intervertebral disc of a subject and, after the application thereof, a crosslinking agent is brought into contact with at least a part of the surface of the composition, and which has fluidity upon the application thereof.

7. The composition according to claim 6, which is applied to the nucleus pulposus site of the intervertebral disc.

8. The composition according to claim 7, wherein the application to the nucleus pulposus site is the filling of the composition into a defective site of the nucleus pulposus.

9. The composition according to any one of claims 6 to 8, wherein the crosslinking agent is a divalent or higher valent metal ion compound.

10. The composition according to any one of claims 1 to 9, wherein the monovalent metal salt of alginic acid is a low endotoxin monovalent metal salt of alginic acid.

11. The composition according to any one of claims 1 to 10, wherein the monovalent metal salt of alginic acid has a weight average molecular weight (absolute molecular weight) of 80,000 or more, as measured by a GPC-MALS method.

12. The composition according to any one of claims 1 to 11, wherein the concentration of the monovalent metal salt of alginic acid is 0.5 w/w % to 5.0 w/w %.

13. The composition according to any one of claims 1 to 12, which is for use in the treatment, prevention or recurrence suppression of intervertebral disc degeneration and/or intervertebral disc damage.

14. The composition according to claim 13, wherein the intervertebral disc degeneration and/or the intervertebral disc damage are at least one selected from the group consisting of intervertebral disc herniation, discopathy, degenerative spondylolisthesis, pyogenic discitis, spondylosis deformans, spinal canal stenosis, and intervertebral disc damage.

15. A composition for regeneration of an intervertebral disc, comprising a monovalent metal salt of alginic acid and human bone marrow-derived high-purity mesenchymal stem cells, wherein the composition is applied to the nucleus pulposus site of the intervertebral disc of a subject, in a state in which the composition has fluidity.

16. The composition for regeneration of an intervertebral disc according to claim 15, which is applied to the nucleus pulposus site of the intervertebral disc of a subject, in a state in which the composition has fluidity, and is then used without bringing a crosslinking agent into contact with the composition.

17. The composition according to claim 15 or 16, wherein regeneration of the nucleus pulposus of an intervertebral disc is promoted via activation of nucleus pulposus cells by the human bone marrow-derived high-purity mesenchymal stem cells and/or differentiation of the human bone marrow-derived high-purity mesenchymal stem cells into nucleus pulposus cells.

18. The composition according to any one of claims 15 to 17, wherein the human bone marrow-derived high-purity mesenchymal stem cells are in an undifferentiated state upon the application thereof and/or are applied without treatments of induction of differentiation.

19. The composition according to any one of claims 15 to 18, wherein the human bone marrow-derived high-purity mesenchymal stem cells are a cell population of LNGFR (CD271)-positive, or LNGFR (CD271) and Thy-1 (CD90) double-positive, rapidly proliferating mesenchymal stem cell clones, said cell population satisfying at least one of the following characteristics (a) and (b):
(a) the coefficient of variation of forward scattered light in flow cytometry is 40% or less; and
(b) the average cell size is 20 lam or less.

20. The composition according to any one of claims 15 to 19, wherein the human bone marrow-derived high-purity mesenchymal stem cells are a cell population of rapidly proliferating mesenchymal stem cell clones that are separated using, as an indicator, the feature that the cells are LNGFR (CD271)-positive, or LNGFR (CD271) and Thy-1 (CD90) double-positive cells, said cell population satisfying at least one of the following characteristics (a) and (b):
(a) the coefficient of variation of forward scattered light in flow cytometry is 40% or less; and
(b) the average cell size is 20 μm or less.

21. The composition according to any one of claims 15 to 20, wherein the monovalent metal salt of alginic acid is a low endotoxin monovalent metal salt of alginic acid.

22. The composition according to any one of claims 15 to 21, wherein the monovalent metal salt of alginic acid has a weight average molecular weight (absolute molecular weight) of 80,000 or more, as measured by a GPC-MALS method.

23. The composition according to any one of claims 15 to 22, wherein the concentration of the monovalent metal salt of alginic acid is 0.5 w/w % to 5.0 w/w %.

24. The composition according to any one of claims 15 to 23, which is for use in the treatment, prevention or recurrence suppression of intervertebral disc degeneration and/or intervertebral disc damage.

25. The composition according to claim 24, wherein the intervertebral disc degeneration and/or the intervertebral disc damage are at least one selected from the group consisting of intervertebral disc herniation, discopathy, degenerative spondylolisthesis, pyogenic discitis, chronic low back pain, spondylosis deformans, spinal canal stenosis, lumbar spinal stenosis, intervertebral disc herniation associated with lumbar spinal stenosis (combined lumbar spinal stenosis), and intervertebral disc damage.

26. The composition according to claim 24 or 25, wherein the intervertebral disc degeneration and/or the intervertebral disc damage are associated with chronic low back pain.

27. The composition according to any one of claims 15 to 23, which is used to suppress intervertebral disc pain.
